(19) United States
(12) Patent Application Publication
(10) Pub. No.: US 2010/0317625 A1
(43) Pub. Date: Dec. 16, 2010

(54) NOVEL SULFONAMIDOMETHYLPHOSPHONATE INHIBITORS OF BETA-LACTAMASE

(76) Inventors: Frank Dininno, Barnegat, NJ (US); Milton L. Hammond, Somerville, NJ (US); Kevin Dykstra, West Milford, NJ (US); Seongkon Kim, Holmdel, NJ (US); Qiang Tan, Westfield, NJ (US); Katherine Young, Metuchen, NJ (US); Jeffrey Donald Hermes, Warren, NJ (US); Stephane Raeppel, St. Lazare (CA); Michael Mannion, Montreal (CA); Nancy Z. Zhou, Kirkland (CA); Frederic Gaudette, Verdun (CA); Arkadii Vaisburg, Kirkland (CA); Jubrail Rahil, Dollard Des Ormeaux (CA); Nafsika Georgopapadakou, Kirkland (CA)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 12/301,793

(22) PCT Filed: May 21, 2007
(86) PCT No.: PCT/US2007/011977

§ 371 Date: Jun. 29, 2010

Related U.S. Application Data
(60) Provisional application No. 60/802,102, filed on May 22, 2006.

(51) Int. Cl. A61K 31/675; C07F 9/6553; A61K 31/67; C07F 9/6561; C07F 9/6539; C07F 9/655; A61K 31/665; A61P 31/04

(52) U.S. Cl. 514/89; 549/6; 514/96; 546/25; 548/119; 514/92; 514/95; 549/220; 514/100

(57) ABSTRACT

This invention provides novel β-lactamase inhibitors of the aryl- and heteroaryl-sulfonamidomethylphosphonate monoester class. The compounds inhibit three classes of β-lactamases and synergize the antibacterial effects of β-lactam antibiotics (e.g., imipenem and ceftazimdime) against those micro-organisms normally resistant to the β-lactam antibiotics as a result of the presence of the β-lactamases.

### BACKGROUND OF THE INVENTION

Bacterial antibiotic resistance has become one of the most important threats to modern health care. Cohen, Science 257:1051-1055 (1992) discloses that infections caused by resistant bacteria frequently result in longer hospital stays, higher mortality and increased cost of treatment. Neu, Science 257:10641073 (1992) discloses that the need for new antibiotics will continue to escalate because bacteria have a remarkable ability to develop resistance to new agents rendering them quickly ineffective.

The present crisis has prompted various efforts to elucidate the mechanisms responsible for bacterial resistance, Coulton et al., Progress in Medicinal Chemistry 31:297-349 (1994) teaches that the widespread use of penicillins and cephalosporins has resulted in the emergence of β lactamases, a family of bacterial enzymes that catalyze the hydrolysis of the β-lactam ring common to numerous presently used antibiotics. More recently, Dudley, Pharmacotherapy 15: 9S-14S (1995) has disclosed that resistance mediated by β-lactamases is a critical aspect at the core of the development of bacterial antibiotic resistance. Clavulanic acid, which is a metabolite of Streptomyces clavuligerus, and two semi-synthetic inhibitors, sulbactam and tazobactam are presently available semi-synthetic or natural product β-lactamase inhibitors. U.S. Pat. No. 6,472,406, incorporated herein in its entirety, discloses certain synthetic β-lactamase inhibitors.

The availability of only a few β-lactamase inhibitors, however, is insufficient to counter the constantly increasing diversity of β-lactamases, for which a variety of novel and distinct inhibitors has become a necessity. There is, therefore, a need for the ability to identify new β-lactamase inhibitors.

This invention relates to novel beta-lactamase inhibitors and their use against bacterial antibiotic resistance. More particularly, the invention relates to compositions and methods for overcoming bacterial antibiotic resistance.

### SUMMARY OF THE INVENTION

This invention provides novel β-lactamase inhibitors of the aryl- and heteroaryl-sulfonamidomethylphosphonate monoester class having specific amide groups. The compounds inhibit three classes of β-lactamases and synergize the antibacterial effects of β-lactam antibiotics (e.g., imipenem and ceftazidime) against those micro-organisms normally resistant to the β-lactam antibiotics as a result of the presence of the β-lactamases. This invention also relates to the combination of the claimed compounds with all relevant β-lactam antibiotics to extend the spectrum of antimicrobial activity of the antibiotic against β-lactamase producing bacteria such as Pseudomonas spp and in particular Acinitobacter baumanii. The invention further relates to compositions containing compounds of this invention and a pharmaceutically acceptable carrier or carriers. It also relates to methods for treating bacterial infections and inhibiting bacterial growth using the compounds or compositions of this invention. This and other aspects of the invention are realized upon consideration of the specification in its entirety.

### DETAILED DESCRIPTION OF THE INVENTION

The present invention is directed to novel compounds of Formula I:

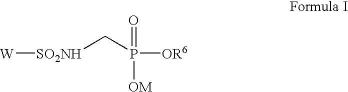

or a pro-drug or pharmaceutically acceptable salt thereof, wherein:
W represents:

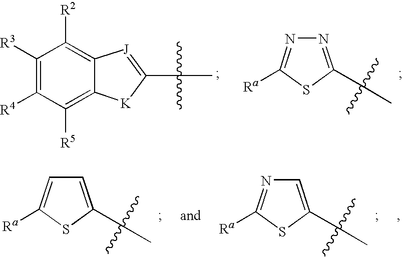

R^{a} represents: (CH₂)ₙKR^{aa}, or R^{aa};
R^{aa} represents:

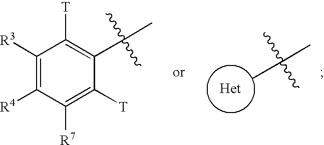

J represents N or CR¹;
K represents O, S, or NR¹;
Het represents a 5-6 membered nitrogen containing heterocycle substituted with 0 to 4 groups of R²;
T represents hydrogen, halogen, OR¹ or Cl₁₋₆ alkyl;
M is a negative charge, H, or a pharmaceutically acceptable metal or ammonium salt, and provided that when W contains a moiety with multiple positive charges, there is an appropriate number of L^{⊖} present to provide overall neutrality;
R¹ independently represents hydrogen, or C₁₋₆ alkyl;
R² and R⁵ independently represent hydrogen, halogen, cyano, —OR¹ or C₁₋₆ alkyl;
R³, R⁴, and R² independently represent hydrogen, halogen, cyano, —OR¹ C₁₋₆ alkyl, or —Xₘ—Yₘ—Z*ₘ—R⁸;
X and Y independently are bond, O, (C═O), SO₂, (CH₂)ₙ, —CH₂)ₙNR¹C(O)—, —CH₂)ₙS—, —(CH₂)ₙNR^{x}—;
Z* is (CH₂)ₙ which may be substituted with one to four R^{b},
R⁶ represents C₆₋₁₀ aryl, or C₅₋₁₀ heteroaryl, said aryl and heteroaryl optionally substituted;
R⁸ represents H, halogen, —N(R^{c})₂, —C(O)R⁵, —NR¹(CH₂)ₙC₅₋₁₀ aryl, —NR¹(CH₂)ₙC₅₋₁₀ heterocyclyl, (CH₂)ₙC₅₋₁₀ heterocyclyl, or (CH₂)ₙC₅₋₁₀ aryl, said heterocyclyl and aryl optionally substituted with one to four R^{b};

each R^{b} independently represents halogen; —CN; —NO₂; phenyl; —NHSO₂R^{c}; —OR^{c}, —SR^{c}; —N(R^{c})₂; —N⁺(R^{c})₃; —C(O)N(R^{c})₂; —SO₂N(R^{c})₂; heteroaryl; heteroarylium; —CO₂R^{c}; —C(O)R^{c}; —OC(O)R^{c}; —NHC(O)R^{c}; —NHC(O)₂R^{c}; guanidinyl; carbamimidoyl or ureido, said phenyl and heteroaryl optionally substituted;

each R^{c} independently represents hydrogen, a —C₁₋₆ straight or branched-chain alkyl group, a —C₃-C₆ cycloalkyl group or C₆₋₁₀ aryl, said aryl optionally substituted with one to four groups of halogen; —CN; —NO₂; phenyl; —NHSO₂R^{j}; —OR¹, —SR^{j}; —N(R^{j})₂; —N⁺(R^{j})₃; —C(O)N(R^{j})₂; —SO₂N(R^{j})₂; heteroaryl; heteroarylium; formamidinyl, —CO₂R^{j}; —C(O)R^{j}; —OC(O)R^{j}; —NHC(O)R^{j}; —NHC(O)₂R^{j}; guanidinyl; carbamimidoyl or ureido, said phenyl and heteroaryl optionally substituted, wherein Rj is selected from the group consisting of hydrogen, a —C₁₋₆ straight or branched-chain alkyl group, a —C₃-C₆ cycloalkyl group or C₆₋₁₀ aryl;

R^{x} represents hydrogen or a C₁₋₈ straight- or branched-alkyl chain, optionally interrupted by one or two of O, S, SO, SO₂, NR^{w}, N⁺R^{c}R^{w}, or —C(O)—, said alkyl chain being unsubstituted or substituted with one to four of halogen, CN, NO₂, —N₃, OR^{w}, SR^{w}, SOR^{w}, SO₂R^{w}, NR^{c}R^{w}, N⁺(R^{c})₂R^{w}, Q, —C(O)—R^{w}, C(O)NR^{c}R^{w}, SO₂NR^{c}R^{w}, CO₂R^{w}, OC(O)R^{w}, OC(O)NR^{c}R^{w}, NR^{c}C(O)R^{w}, NR^{c}C(O)NR^{c}R^{w}, phenyl, napthyl, heteroaryl, or heterocyclic group said phenyl, heteroaryl, and heterocyclic group optionally substituted with from one to four R^{b} groups or with one to two C₁₋₃ straight- or branched-chain alkyl groups, said alkyl groups being unsubstituted or substituted with one to four R^{b} groups; and

each R^{w} independently represents hydrogen; —C₁₋₆ straight- or branched-chain alkyl, unsubstituted or substituted with one to four R^{b} groups; —C₃₋₆ cycloalkyl optionally substituted with one to four R^{b} groups; phenyl optionally substituted with one to four R^{b} groups, or heteroaryl optionally substituted with one to four R^{b} groups;

m represents 0 to 1; n represents 0 to 6; wherein it is understood that when a value is zero, a bond exists.

The invention further relates to bacterial antibiotic resistance. More particularly, the invention relates to compositions and methods for overcoming bacterial antibiotic resistance. The patents and publications identified in this specification indicate the knowledge in this field and are hereby incorporated by reference in their entirety. In the case of inconsistencies, the present disclosure will prevail.

The invention provides novel β-lactamase inhibitors, which are structurally unrelated to the natural product and semi-synthetic β-lactamase inhibitors presently available, and which do not require a β-lactam pharmacophore. Certain embodiments of these new inhibitors may also bind bacterial DD-peptidases, and thus may potentially act both as β-lactamase inhibitors and as antibiotic agents.

For purposes of the present invention, the following definitions will be used:

As used herein, the term “β-lactamase inhibitor” is used to identify a compound having a structure as defined herein, which is capable of inhibiting β-lactamase activity. Inhibiting β-lactamase activity means inhibiting the activity of a class A, C, or D β-lactamase. Preferably, for antimicrobial applications such inhibition should be at a 50% inhibitory concentration below 100 micrograms/mL, more preferably below 30 micrograms/mL and most preferably below 10 micrograms/mL. The terms “class A”, “class C”, and “class D” β-lactamases are understood by those skilled in the art and can be found described in Waley, The Chemistry of β-lactamase, Page Ed., Chapman & Hall, London, (1992) 198-228.

In some embodiments of the invention, the β-lactamase inhibitor may also be capable-of acting as an antibiotic agent by inhibiting bacterial cell-wall cross-linking enzymes. Thus, the term β-lactamase inhibitor is intended to encompass such dual-acting inhibitors. In certain preferred embodiments, the β-lactamase inhibitor may be capable of inhibiting D-alanyl-D-alanine-carboxypeptidases/transpeptidases (hereinafter DD-peptidases). The term “DD-peptidase” is used in its usual sense to denote penicillin-binding proteins involved in bacterial cell wall biosynthesis (see, e.g., Ghysen, Prospect. Biotechnol. 128:67-9a (1987)). In certain particularly preferred embodiments, the D-alanyl-D-alanine-carboxypeptidase/transpeptidase, which may be inhibited is the Streptomyces R61 DD-peptidase. This enzyme has conservation of active site mechanism with bacterial signal peptidases (see, e.g., Black et al., Current Pharmaceutical Design 4:133-1.54 (1998); Dalbey et al., Protein Science 6:1129-1138 (1997)). It is, therefore, possible that the β-lactamase inhibitors of the invention may also be capable of inhibition of bacterial signal peptidases.

As used herein, the term “β-lactamase” denotes a protein capable of inactivating a β-lactam antibiotic. In one preferred embodiment, the β-lactamase is an enzyme which catalyzes the hydrolysis of the β-lactam ring of a β-lactam antibiotic. In certain preferred embodiments, the β-lactamase is microbial. In certain other preferred embodiments, the β-lactamase is a serine β-lactamase. Examples of such preferred β-lactamases are well known and are disclosed in, e.g., Waley, The Chemistry of β-Lactamase, Page Ed., Chapman & Hall, London, (1992) 198-228. In particularly preferred embodiments, the β-lactamase is a class C β-lactamase of Enterobacter cloacae P99 (hereinafter P99 β-lactamase), or a class A β-lactamase of the TEM-1 plasmid (hereinafter TEM β-lactamase).

When any variable (e.g. aryl, heterocycle, R¹, R⁵ etc.) occurs more than one time in any constituent, its definition on each occurrence is independent at every other occurrence. Also, combinations of substituents/or variables are permissible only if such combinations result in stable compounds.

As used herein, the term “organism” refers to any multicellular organism. Preferably, the organism is an animal, more preferably a mammal, and most preferably a human

For simplicity, chemical moieties are defined and referred to throughout primarily as univalent chemical moieties (e.g., alkyl, aryl, etc.). Nevertheless, such terms are also used to convey corresponding multivalent moieties under the appropriate structural circumstances clear to those skilled in the art. For example, while an “alkyl” moiety generally refers to a monovalent radical (e.g. CH₃—CH₂—), in certain circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene.) All atoms are understood to have their normal number of valences for bond formation (i.e., 4 for carbon, 3 for N, 2 for 0, and 2, 4, or 6 for S, depending on the oxidation state of the S). On occasion a moiety may be defined, for example, as (A)ₐ-B-, wherein a is 0 or 1. In such instances, when a is 0 the moiety is B- and when a is 1 the moiety is A-B-. Also, a number of moieties disclosed herein exist in multiple tautomeric forms, all of which are intended to be encompassed by any given tautomeric structure.

The term “alkyl” as employed herein refers to straight and branched chain aliphatic groups having from 1 to 12 carbon atoms, preferably 1-8 carbon atoms, more preferably 1-6 carbon atoms, which is optionally substituted with one, two or three substituents. Unless otherwise specified, the alkyl group may be saturated, unsaturated, or partially unsaturated. As used herein, therefore, the term “alkyl” is specifically intended to include alkenyl and alkynyl groups, as well as saturated alkyl groups. Preferred alkyl groups include, without limitation, methyl, ethyl, propyl, isopropyl, butyl, tert-butyl, isobutyl, pentyl, hexyl, vinyl, alkyl, isobutenyl, ethynyl, and propynyl.

As employed herein, a “substituted” alkyl, cycloalkyl, aryl, or heterocyclic group is one having between one and about four, preferably between one and about three, more preferably one or two, non-hydrogen substituents. Suitable substituents include, without limitation, halo, hydroxy, nitro, haloalkyl, alkyl, alkaryl, aryl, aralkyl, alkoxy, aryloxy, amino, acylamino, alkylcarbamoyl, arylcarbamoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups.

The term “cycloalkyl” as employed herein includes saturated and partially unsaturated cyclic hydrocarbon groups having 3 to 12, preferably 3 to 8 carbons, wherein the cycloalkyl group additionally is optionally substituted. Preferred cycloalkyl groups include, without limitation, cyclopropyl, cyclobutyl, cyclopentyl, cyclopentenyl, cyclohexyl, cyclohexenyl, cycloheptyl, and cyclooctyl.

An “aryl” group is a C₆-C₁₄ aromatic moiety comprising one to three aromatic rings, which is optionally substituted. Preferably, the aryl group is a C₆-C₁₀ aryl group. Preferred aryl groups include, without limitation, phenyl, naphthyl, anthracenyl, and fluorenyl. An “aralkyl” or “arylalkyl” group comprises an aryl group covalently linked to an alkyl group, either of which may independently be optionally substituted or unsubstituted. Preferably, the aralkyl group is C₁₋₆alkyl(C6-10)aryl including, without limitation, benzyl, phenethyl, and naphthylmethyl. An “alkaryl” or “alkylaryl” group is an aryl group having one or more alkyl substituents. Examples of alkaryl groups include, without limitation, tolyl, xylyl, mesityl, ethylphenyl, tert-butylphenyl, and methylnaphthyl.

The term heterocycle, heterocyclyl, or heterocyclic, as used herein, represents a stable 5- to 7-membered monocyclic or stable 8- to 11-membered bicyclic heterocyclic ring which is either saturated or unsaturated, and which consists of carbon atoms and from one to four heteroatoms selected from the group consisting of N, O, and S, and including any bicyclic group in which any of the above-defined heterocyclic rings is fused to a benzene ring. The heterocyclic ring may be attached at any heteroatom or carbon atom which results in the creation of a stable structure. The term heterocycle or heterocyclic includes heteroaryl moieties. Examples of such heterocyclic elements include, but are not limited to, azepinyl, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, 1,3-dioxolanyl, furyl, imidazolidinyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, oxazolyl, 2-oxopiperazinyl, 2-oxopiperidinyl, 2-oxopyrrolidinyl, piperidyl, piperazinyl, pyridyl, pyrazinyl, pyrazolidinyl, pyrazolyl, pyridazinyl, pyrimidinyl, pyrrolidinyl, pyrrolyl, quinazolinyl, quinolinyl, quinoxalinyl, tetrahydrofuryl, tetrahydroisoquinolinyl, tetrahydroquinolinyl, thiamorpholinyl, thiamorpholinyl sulfoxide, thiazolyl, thiazolinyl, thienofuryl, thienothienyl, and thienyl. An embodiment of the examples of such heterocyclic elements include, but are not limited to, azepinyl, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, furyl, imidazolidinyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, oxazolyl, 2-oxopiperazinyl, 2-oxopiperidinyl, 2-oxopyrrolidinyl, piperidyl, piperazinyl, pyridyl, 2-pyridinonyl, pyrazinyl, pyrazolidinyl, pyrazolyl, pyridazinyl, pyrimidinyl, pyrrolidinyl, pyrrolyl, quinazolinyl, quinolinyl, quinoxalinyl, tetrahydrofuryl, tetrahydroisoquinolinyl, tetrahydroquinolinyl, thiamorpholinyl, thiamorpholinyl sulfoxide, thiazolyl, thiazolinyl, thienofuryl, thienothienyl, thienyl and triazolyl.

In certain preferred embodiments, the heterocyclic group is a heteroaryl group. As used herein, the term “heteroaryl” refers to groups having 5 to 14 ring atoms, preferably 5, 6, 9, or 10 ring atoms; having 6, 10, or 14 π electrons shared in a cyclic array; and having, in addition to carbon atoms, between one and about three heteroatoms selected from the group consisting of N, 0, and S. Preferred heteroaryl groups include, without limitation, thienyl, benzothienyl, furyl, benzofuryl, dibenzofuryl, pyrrolyl, imidazolyl, pyrazoiyl, pyridyl, pyrazinyl, pyrimidinyl, indolyl, quinolyl, isoquinolyl, quinoxalinyl, tetrazolyl, oxazolyl, thiazolyl, and isoxazolyl.

In certain other preferred embodiments, the heterocyclic group is fused to an aryl or heteroaryl group. Examples of such fused heterocycles include, without limitation, tetrahydroquinolinyl and dihydrobenzofuranyl.

A moiety that is substituted is one in which one or more hydrogens have been independently replaced with another chemical substituent. As a non-limiting example, substituted phenyls include 2-fluorophenyl, 3,4-dichlorophenyl, 3-chloro-4-fluoro-phenyl, 2,4-fluor-3-propylphenyl. As another non-limiting example, substituted n-octyls include 2,4 dimethyl-5-ethyl-octyl and 3-cyclopentyloctyl. Included within this definition are methylenes (—CH₂—) substituted with oxygen to form carbonyl (—CO—).

Unless otherwise stated, as employed herein, when a moiety (e.g., cycloalkyl, hydrocarbyl, aryl, alkyl, heteroaryl, heterocyclic, urea, etc.) is described as “optionally substituted” it is meant that the group optionally has from one to four, preferably from one to three, more preferably one or two, non-hydrogen substituents. Suitable substituents include, without limitation, halo, hydroxy, oxo (e.g., an annular —CH— substituted with oxo is —C(O)—), nitro, halohydrocarbyl, hydrocarbyl, aryl, aralkyl, alkoxy, aryloxy, amino, acylamino, alkylcarbamoyl, arylcarbamoyl, aminoalkyl, acyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups. Preferred substituents, which are themselves not further substituted (unless expressly stated otherwise) are:
(a) halo, cyano, oxo, carboxy, formyl, nitro, amino, amidino, guanidino, and
(b) C₁-C₆ alkyl or alkenyl or arylalkyl imino, carbamoyl, azido, carboxamido, mercapto, hydroxy, hydroxyalkyl, alkylaryl, arylalkyl, C₁-C₈ alkyl, SO₂CF₃, CF₃, SO₂Me, C₁-C₈ alkenyl, C₁-C₈ alkoxy, C₁-C₈ alkoxycarbonyl, aryloxycarbonyl, C₂-C₈ acyl, C₂-C₈ acylamino, C₁-C₈ alkylthio, arylalkylthio, arylthio, C₁-C₈alkylsulfinyl, arylalkylsulfnyl, arylsulfnyl, C₁-C₈ alkylsulfonyl, arylalkylsulfonyl, arylsulfonyl, C₀-C₆ N-alkylcarbamoyl, C₂-C₁₅ N,N dialkylcarbamoyl, C₃-C₇ cycloalkyl, aroyl, aryloxy, arylalkyl ether, aryl, aryl fused to a cycloalkyl or heterocycle or another aryl ring, C₃-C₇ heterocycle, or any of these rings fused or spiro-fused to a cycloalkyl, heterocyclyl, or aryl, wherein each of the foregoing is further optionally substituted with one more moieties listed in (a), above.

The term “halogen” or “halo” as employed herein refers to chlorine, bromine, fluorine, or iodine

The term “acylamino” refers to an amide group attached at the nitrogen atom. The term “carbamoyl” refers to an amide group attached at the carbonyl carbon atom. The nitrogen atom of an acylamino or carbamoyl substituent may be additionally substituted. The term “sulfonamido” refers to a sulfonamide substituent attached by either the sulfur or the nitrogen atom. The term “amino” is meant to include NH₂, alkylamino, arylamino, and cyclic amino groups.

The term “ureido” as employed herein refers to a substituted or unsubstituted urea moiety.

The term “heterocycloalkyl” refers to a cycloalkyl group (nonaromatic) in which one of the carbon atoms in the ring is replaced by a heteroatom selected from O, S or N, and in which up to three additional carbon atoms may be replaced by hetero atoms.

The term “heteroatom” means O, S or N, selected on an independent basis.

Alkoxy refers to C₁-C₄ alkyl-O—, with the alkyl group optionally substituted as described herein.

When a functional group is termed “protected”, this means that the group is in modified form to preclude undesired side reactions at the protected site. Suitable protecting groups for the compounds of the present invention will be recognized from the present application taking into account the level of skill in the art, and with reference to standard textbooks, such as Greene, T. W. et al. Protective Groups in Organic Synthesis Wiley, New York (1991). Examples of suitable protecting groups are contained throughout the specification.

An embodiment of this invention is realized when T is alkyl and all other variables are as originally described. Another embodiment is realized when T is hydrogen and all other variables are as originally described.

Another embodiment of this invention is realized when W is

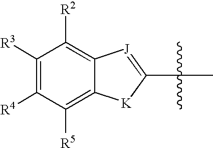

and all other variables are as originally described. A sub-embodiment of this invention is realized when J is CR¹ and K is S. Still another sub-embodiment is realized when J is N and K is S.

Still another embodiment of this invention is realized when W is selected from the group consisting of:

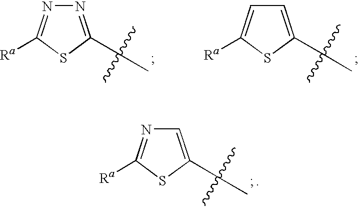

Another embodiment of this invention is realized when R^{a} represents R^{aa} and all other variables are as originally described.

Another embodiment of this invention is realized when at least one of R³, R⁴, and R⁷ is —Xₘ—Yₘ—Z*ₘ—R⁸ and all other variables are as originally described.

Another embodiment of this invention is realized when R³ and R⁴, both are —Xₘ—Yₘ—Z*ₘ—R⁸ and all other variables are as originally described.

Another embodiment of this invention is realized when X is O and all other variables are as originally described.

Another embodiment of this invention is realized when Y is (CH₂)ₙNR¹CO⁻, —(CH₂)ₙS—, or (CH₂)ₙNR^{x} and all other variables are as originally described.

Another embodiment of this invention is realized when X is O, Y is a bond, Z* is (CH₂)ₙ, and all other variables are as originally described.

Another embodiment of this invention is realized when X is O, Y is (CH₂)ₙNR¹CO—Z* is (CH₂)ₙ, and all other variables are as originally described.

Still another embodiment of this invention is realized when R⁸ is (CH₂)ₙC₅₋₁₀ heterocyclyl, or (CH₂)ₙC₅₋₁₀ aryl and all other variables are as originally described. A sub-embodiment of this invention is realized when R⁸ is (CH₂)ₙC₅₋₁₀ aryl.

Another embodiment of this invention is realized when R⁶ is C₆₋₁₀ aryl optionally substituted, and all other variables are as originally described.

Another embodiment of this invention is realized when R⁶ is C₅₋₁₀ heteroaryl optionally substituted, and all other variables are as originally described.

A preferred embodiment of this invention is a compound of formula II:

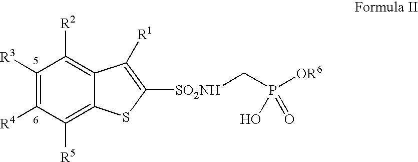

or a pharmaceutically acceptable salt thereof, wherein:
R¹, R², R³. R⁴, R⁵, R⁶ are as previously described.

Another preferred embodiment of this invention is realized when O—R⁶ is selected from the group consisting of,

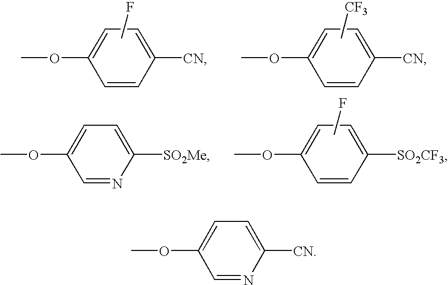

The compounds of this invention can be combined with beta-lactam antibiotics such as imipenem, Primaxin®, Amoxicillin, Ticarcillin, Ampicillin, Cefoperazone, Piperacillin, and ceftazidime. Thus, another aspect of this invention is realized when the compound of this invention are co-administered with a beta-lactam antibiotic.

Examples of compounds of this invention are:
{2-[({[[4-cyano-3-(trifluoromethyl)phenoxy](hydroxy)phosphoryl]methyl}amino)sulfonyl]-4,7-difluoro-1-benzothiene-5,6-diyl}bis(oxyethane-2,1-diyliminocarbonylbenzene-4,1,2-triyl)tetraacetate;
{2-[({[[4-cyano-3-fluorophenoxy](hydroxy)phosphoryl]methyl}amino)sulfonyl]-4,7-difluoro-1-benzothiene-5,6-diyl}bis(oxyethane-2,1-diyliminocarbonylbenzene-4,1,2-triyl)tetraacetate;
{2-[({[[4-cyano-3-fluorophenoxy](hydroxy)phosphoryl]methyl}amino)sulfonyl]-4,7-dichloro-1-benzothiene-5,6-diyl}bis(oxyethane-2,1-diyliminocarbonylbenzene-4,1,2-triyl)tetraacetate;
{2-[({[(4-cyano-3-fluorophenoxy)(hydroxy)phosphoryl]methyl}amino)sulfonyl]-1-benzothiene-5,6-diyl}bis(oxyethane-2,1-diyliminocarbonylbenzene-3,1,2-triyl)tetraacetate;
{2-[({[[4-cyano-3-fluorophenoxy](hydroxy)phosphoryl]methyl}amino)sulfonyl]-1-benzothiene-5,6-diyl}bis(oxyethane-2,1-diyliminocarbonylbenzene-4,1,2-triyl)tetraacetate;
{2-[({[[4-cyano-3-(trifluoromethyl)phenoxy](hydroxy)phosphoryl]methyl}amino)sulfonyl]-1-benzothiene-5,6-diyl}bis(oxyethane-2,1-diyliminocarbonylbenzene-4,1,2-triyl)tetraacetate;
{2-[({[[(6-cyanopyridin-3-yl)oxy](hydroxy)phosphoryl]methyl}amino)sulfonyl]-1-benzothiene-5,6-diyl}bis(oxyethane-2,1-diyliminocarbonylbenzene-4,1,2-triyl)tetraacetate;
{2-[({[[4-cyano-3-fluorophenoxy](hydroxy)phosphoryl]methyl}amino)sulfonyl]-1-benzothiene-5,6-diyl}bis(oxypropane-2,1-diyliminocarbonylbenzene-4,1,2-triyl)tetraacetate;
{2-[({[[4-cyano-3-(trifluoromethyl)phenoxy](hydroxy)phosphoryl]methyl}amino)sulfonyl]-1-benzothiene-5,6-diyl}bis(oxypropane-2,1-diyliminocarbonylbenzene-4,1,2-triyl)tetraacetate;
2-(acetyloxy)-4-({[3-({6-(4-{[3,4-bis(acetyloxy)benzoyl]amino}butoxy)-2-[({[(4-cyano-3-fluorophenoxy)-(hydroxy)phosphoryl]methyl}amino)sulfonyl]-1-benzothien-5-yl}oxy)propyl]amino}carbonyl)phenyl acetate;
2-(acetyloxy)-4-({[3-({6-(4-{[3,4-bis(acetyloxy)benzoyl]amino}propoxy)-2-[({[(4-cyano-3-fluorophenoxy)-(hydroxy)-phosphoryl]methyl}amino)sulfonyl]-1-benzothien-5-yl}oxy)butyl]amino}carbonyl)phenyl acetate;
{2-[({[[4-cyano-3-fluorophenoxy](hydroxy)phosphoryl]methyl}amino)sulfonyl]-4,7-difluoro-1-benzothiene-5,6-diyl}bis(oxypropane-2,1-diyliminocarbonylbenzene-4,1,2-triyl)tetraacetate;
{2-[({[[4-cyano-3-fluorophenoxy](hydroxy)phosphoryl]methyl}amino)sulfonyl]-3-methyl-1-benzothiene-5,6-diyl}bis(oxypropane-2,1-diyliminocarbonylbenzene-4,1,2-triyl)tetraacetate;
{2-[({[[4-cyano-3-fluorophenoxy](hydroxy)phosphoryl]methyl}amino)sulfonyl]-3-ethyl-1-benzothiene-5,6-diyl}bis(oxypropane-2,1-diyliminocarbonylbenzene-4,1,2-triyl)tetraacetate;
{2-[({[[4-cyano-3-fluorophenoxy](hydroxy)phosphoryl]methyl}amino)sulfonyl]-4,7-difluoro-3-ethyl-1-benzothiene-5,6-diyl}bis(oxypropane-2,1-diyliminocarbonylbenzene-4,1,2-triyl)tetraacetate;
{2-[({[[4-cyano-3-fluorophenoxy](hydroxy)phosphoryl]methyl}amino)sulfonyl]-4,7-difluoro-3-ethyl-1-benzothiene-5,6-diyl}bis(oxyethane-2,1-diyliminocarbonylbenzene-4,1,2-triyl)tetraacetate;
(4-{5-[({[(4-cyano-3-fluorophenoxy)(hydroxy)phosphoryl]methyl}amino)sulfonyl]-2-thienyl}-1,2-phenylene)bis(oxypropane-3,1-diyliminocarbonylbenzene-4,1,2-triyl)tetraacetate;
(4-{5-[({[(4-cyano-3-fluorophenoxy)(hydroxy)phosphoryl]methyl}amino)sulfonyl]-2-thienyl}-3-methyl-1,2-phenylene)bis(oxypropane-3,1-diyliminocarbonylbenzene-4,1,2-triyl)tetraacetate;
4-cyano-3-fluorophenyl hydrogen {[({5,6-bis[2-(1-naphthoylamino)ethoxy]-1-benzothien-2-yl}sulfonyl)amino]-methyl}phosphonate;
4-cyano-3-fluorophenyl hydrogen {[({5,6-bis[2-(2-naphthoylamino)ethoxy]-1-benzothien-2-yl}sulfonyl)amino]-methyl}phosphonate;
4-cyano-3-fluorophenyl hydrogen {[({5,6-bis[2-(benzoylamino)ethoxy]-1-benzothien-2-yl}sulfonyl)amino]-methyl}phosphonate;
4-cyano-3-fluorophenyl hydrogen {[({5,6-bis[2-(3,4-difluorobenzoylamino)ethoxy]-1-benzothien-2-yl}-sulfonyl)amino]methyl}phosphonate;
{2-[({[(4-cyano-3-fluorophenoxy)(hydroxy)phosphoryl]methyl}amino)sulfonyl]-1-benzothiene-5,6-diyl}bis(oxyethane-2,1-diyliminocarbonyl-4,1-phenylene)diacetate;
{2-[({[(4-cyano-3-fluorophenoxy)(hydroxy)phosphoryl]methyl}amino)sulfonyl]-1-benzothiene-5,6-diyl}bis(oxyethane-2,1-diyliminocarbonyl-3,1-phenylene)diacetate;
{2-[({[(4-cyano-3-fluorophenoxy)(hydroxy)phosphoryl]methyl}amino)sulfonyl]-1-benzothiene-5,6-diyl}bis[oxyethane-2,1-diylimino(2-oxoethane-2,1-diyl)benzene-4,1,2-triyl]tetraacetate;
4-(cyano-3-fluorophenyl hydrogen{5,6-bis[3-(3,4-dihydroxybenzamido)propoxy]benzo[d]thiazole-2-sulfonamido}-methylphosphonate;
4-cyano-3-(trifluoromethyl)phenyl hydrogen ({[(5,6-bis{2-[(3,4-dihydroxybenzoyl)amino]ethoxy}-4,7-difluoro-1-benzothien-2-yl)-sulfonyl]amino}methyl)phosphonate;
4-cyano-3-fluorophenyl hydrogen ({[(5,6-bis{2-[(3,4-dihydroxybenzoyl)amino]ethoxy}-4,7-difluoro-1-benzothien-2-yl)sulfonyl]amino}methyl)phosphonate;
4-cyano-3-fluorophenyl hydrogen ({[(5,6-bis {2-[(3,4-dihydroxybenzoyl)amino]ethoxy}-4,7-dichloro-1-benzothien-2-yl)sulfonyl]amino}methyl)phosphonate;
4-cyano-3-fluorophenyl hydrogen ({[(5,6-bis{2-[(2,3-dihydroxybenzoyl)amino]ethoxy}-1-benzothien-2-yl)-sulfonyl]amino}methyl)phosphonate;
4-cyano-3-(trifluoromethyl)phenyl hydrogen ({[(5,6-bis{2-[(2,3-dihydroxybenzoyl)amino]ethoxy}-1-benzothien-2-yl)-sulfonyl]amino}methyl)phosphonate;
4-cyano-3-fluorophenyl hydrogen ({[(5,6-bis{2-[(3,4-dihydroxybenzoyl)amino]ethoxy}-1-benzothien-2-yl)-sulfonyl]amino}methyl)phosphonate;
4-cyano-3-(trifluoromethyl)phenyl hydrogen ({[(5,6-bis{2-[(3,4-dihydroxybenzoyl)amino]ethoxy}-1-benzothien-2-yl)sulfonyl]amino}methyl)phosphonate;
3-fluoro-4-[(trifluoromethyl)sulfonyl]phenyl hydrogen ({[(5,6-bis{2-[(3,4-dihydroxybenzoyl)amino]ethoxy}-1-benzothien-2-yl)sulfonyl]amino}methyl)phosphonate;
6-cyanopyridin-3-yl hydrogen({[(5,6-bis{2-[(3,4-dihydroxybenzoyl)amino]ethoxy}-1-benzothien-2-yl)sulfonyl]amino}methyl)phosphonate;
4-cyano-3-fluorophenyl hydrogen({[(5,6-bis{2-[(3,4-dihydroxybenzoyl)amino]propoxy}-1-benzothien-2-yl)-sulfonyl]amino}methyl)phosphonate;
4-cyano-3-fluorophenyl hydrogen({[(5,6-bis{2-[(3,4-dihydroxybenzoyl)amino]butoxy}-1-benzothien-2-yl)-sulfonyl]amino}methyl)phosphonate;
4-cyano-3-(trifluoromethyl)phenyl hydrogen ({[(5,6-bis{2-[(2,3-dihydroxybenzoyl)amino]propoxy}-1-benzothien-2-yl)sulfonyl]amino}methyl)phosphonate;
4-cyano-3-fluorophenyl hydrogen ({[(6-{4-[(3,4-dihydroxybenzoyl)amino]butoxy}-5-{3-[(3,4-dihydroxybenzoyl)amino]propoxy}-1-benzothien-2-yl)sulfonyl]amino}methyl)phosphonate;
4-cyano-3-fluorophenyl hydrogen({[(5-{4-[(3,4-dihydroxybenzoyl)amino]butoxy}-6-{3-[(3,4-dihydroxybenzoyl)amino]propoxy}-1-benzothien-2-yl)sulfonyl]amino}methyl)phosphonate;
4-cyano-3-(trifluoromethyl)phenyl hydrogen ({[(5,6-bis{2-[(3,4-dihydroxybenzoyl)amino]propoxy}-4,7-dichloro-1-benzothien-2-yl)sulfonyl]amino}methyl)phosphonate;
4-cyano-3-fluorophenyl hydrogen ({[(5,6-bis {2-[(3,4-dihydroxybenzoyl)amino]propoxy}-4,7-difluoro-1-benzothien-2-yl)sulfonyl]amino}methyl)phosphonate;
4-cyano-3-fluorophenyl hydrogen ({[(5,6-bis{2-[(4-hydroxybenzoyl)amino]ethoxy}-1-benzothien-2-yl)-sulfonyl]amino}methyl)phosphonate;
4-cyano-3-fluorophenyl hydrogen ({[(5,6-bis{2-[(3-hydroxybenzoyl)amino]ethoxy}-1-benzothien-2-yl)-sulfonyl]amino}methyl)phosphonate;
4-cyano-3-fluorophenyl hydrogen ({[(5,6-bis{2-[(3,4-dihydroxybenzoyl)amino]propoxy}-3-methyl-1-benzothien-2-yl)sulfonyl]amino}methyl)phosphonate;
4-cyano-3-fluorophenyl hydrogen ({[(5,6-bis{2-[(3,4-dihydroxybenzoyl)amino]propoxy}-3-ethyl-1-benzothien-2-yl)sulfonyl]amino}methyl)phosphonate;
4-cyano-3-fluorophenyl hydrogen ({[(5,6-bis{2-[(3,4-dihydroxybenzoyl)amino]propoxy}-4,7-difluoro-3-ethyl-1-benzothien-2-yl)sulfonyl]amino}methyl)phosphonate;
4-cyano-3-fluorophenyl hydrogen ({[(5,6-bis{2-[(3,4-dihydroxybenzoyl)amino]ethoxy}-4,7-difluoro-3-ethyl-1-benzothien-2-yl]sulfonyl}amino)methyl)phosphonate;
4-cyano-3-fluorophenyl hydrogen[({[5,6-bis(2-{[(3,4-dihydroxyphenyl)acetyl]amino}ethoxy)-1-benzothien-2-yl]sulfonyl}amino)methyl]phosphonate;
4-cyano-3-fluorophenyl hydrogen[({[5-(3,4-bis{3-[(3,4-dihydroxybenzoyl)amino]propoxy}phenyl)-2-thienyl]sulfonyl}amino)methyl]phosphonate;
4-cyano-3-fluorophenyl hydrogen[({[5-(3,4-bis{3-[(3,4-dihydroxybenzoyl)amino]propoxy}-2-methylphenyl)-2-thienyl]sulfonyl}amino)methyl]phosphonate;
4-Cyano-3-fluorophenyl hydrogen (5-{3,4-bis[3-(3,4-dihydroxybenzamido) propoxy]benzamido}-benzo[b]thiophene-2-sulfonamido)methylphosphonate;
4-cyano-3-fluorophenyl hydrogen (5-((pyridin-2-ylthio)methyl)thiophene-2-sulfonamido)-methylphosphonate;
4-cyano-3-fluorophenyl hydrogen (5-(dimethylamino)benzo[b]thiophene-2-sulfonamido)-methylphosphonate;
4-cyano-3-(trifluoromethyl)phenyl hydrogen (7-cyano-6-(dimethylamino)benzo[b]thiophene-2-sulfonamido)methylphosphonate;
4-cyano-3-(trifluoromethyl)phenyl hydrogen (5-(3,4-dihydroxybenzamido)benzo[b]thiophene-2-sulfonamido)methylphosphonate;
4-cyano-3-fluorophenyl hydrogen (5-(2,3-dihydroxybenzamido)benzo[b]thiophene-2-sulfonamido)methylphosphonate;
4-cyano-3-(trifluoromethyl)phenyl hydrogen (5-(2,3-dihydroxybenzamido)benzo[b]thiophene-2-sulfonamido)methylphosphonate;
4-cyano-3-fluorophenyl hydrogen (5-(3,4-dihydroxybenzamido)benzo[b]thiophene-2-sulfonamido)-methylphosphonate;
Ammounium 4-cyano-3-(trifluoromethyl)phenyl (2-(3,4-dimethoxyphenyl)thiazole-5-sulfonamido)methylphosphonate;
Ammonium 4-cyano-3-fluorophenyl (2-(3,4-dimethoxyphenyl)thiazole-5-sulfonamido)methylphosphonate;
4-cyano-3-fluorophenyl hydrogen (2-(3,4-dihydroxyphenyl)thiazole-5-sulfonamido)methylphosphonate;
4-cyano-3-(trifluoromethyl)phenyl hydrogen (2-(3,4-dihydroxyphenyl)thiazole-5-sulfonamido)methylphosphonate;
Ammonium 4-cyano-3-fluorophenyl (2-(2,3-dimethoxyphenyl)thiazole-5-sulfonamido)methylphosphonate;
Ammonium 4-cyano-3-fluorophenyl (5-(3,4-dimethoxyphenyl)thiophene-2-sulfonamido)methylphosphonate;
Ammonium 4-cyano-3-(trifluoromethyl)phenyl (5-(3,4-dimethoxyphenyl)thiophene-2-sulfonamido)methylphosphonate;
4-cyano-3-(trifluoromethyl)phenyl hydrogen (5-(3,4-dihydroxyphenyl)thiophene-2-sulfonamido)methylphosphonate;
4-cyano-3-fluorophenyl hydrogen (5-(3,4-dihydroxyphenyl)thiophene-2-sulfonamido)methylphosphonate;
Ammonium 4-cyano-3-fluorophenyl (5-(6-methoxypyridin-3-yl)thiophene-2-sulfonamido)methylphosphonate;
Ammonium 4-cyano-3-(trifluoromethyl)phenyl (5-(6-methoxypyridin-3-yl)thiophene-2-sulfonamido)methylphosphonate;
6-cyanopyridin-3-yl hydrogen (5-(6-methoxypyridin-3-yl)thiophene-2-sulfonamido)methylphosphonate;
4-cyano-3-fluorophenyl hydrogen (2-(2,3-dihydroxyphenyl)thiazole-5-sulfonamido)methylphosphonate;
4-cyano-3-(trifluoromethyl)phenyl hydrogen (2-(2,3-dimethoxyphenyl)thiazole-5-sulfonamido)methylphosphonate;
4-cyano-3-(trifluoromethyl)phenyl hydrogen (2-(2,3-dihydroxyphenyl)thiazole-5-sulfonamido)methylphosphonate;
4-cyano-3-fluorophenyl hydrogen (5-(6-oxo-1,6-dihydropyridin-3-yl)thiophene-2-sulfonamido)methylphosphonate;
Ammonium 4-cyano-3-fluorophenyl (5-(2,3-dimethoxyphenyl)thiophene-2-sulfonamido)methylphosphonate;
Ammonium 4-cyano-3-(trifluoromethyl)phenyl (5-(2,3-dimethoxyphenyl)thiophene-2-sulfonamido)methylphosphonate;
4-cyano-3-fluorophenyl hydrogen (5-(2,3-dihydroxyphenyl)thiophene-2-sulfonamido)methylphosphonate;
4-cyano-3-(trifluoromethyl)phenyl hydrogen (5-(2,3-dihydroxyphenyl)thiophene-2-sulfonamido)methylphosphonate;
Ammonium 4-cyano-3-(trifluoromethyl)phenyl (5,6-dimethoxybenzofuran-2-sulfonamido)methylphosphonate;
4-cyano-3-(trifluoromethyl)phenyl hydrogen (5,6-dihydroxybenzofuran-2-sulfonamido)methylphosphonate; and
Ammonium 4-cyano-3-fluorophenyl (5-((3,4-dihydroxyphenylsulfonyl)methyl)thiophene-2-sulfonamido)methylphosphonate
   or pharmaceutically acceptable salts thereof.

In certain preferred embodiments, the β-lactamase inhibitor is a salt of the compound of Formula I or II, the salt preferably being formed by treating the compound of Formula I or II with a base so as to remove the phosphonate hydrogen atom. Non-limiting examples of bases which may be use to deprotonate the compound of Formula (I) or (II) include sodium hydroxide, potassium hydroxide ammonium hydroxide, sodium bicarbonate, potassium bicarbonate, sodium carbonate, and potassium carbonate. Preferably, the counterion thereby introduced is a pharmaceutically acceptable counterion, including without limitation sodium, magnesium, calcium, or ammonium.

Another aspect, the invention provides pharmaceutical compositions comprising a β-lactamase inhibitor of the invention and a pharmaceutically acceptable carrier or diluent. The characteristics of the carrier will depend on the route of administration. As used herein, the term “pharmaceutically acceptable” means a non-toxic material that does not interfere with the effectiveness of the biological activity of the active ingredient(s). The term “physiologically acceptable” refers to a non-toxic material that is compatible with a biological system such as a cell, cell culture, tissue, or organism. Thus, compositions and methods according to the invention may contain, in addition to the inhibitor, diluents, fillers, salts, buffers, stabilizers, solubilizers, and other materials well known in the art. The pharmaceutical composition of the invention may also contain other active factors and/or agents which enhance the inhibition of β-lactamases and/or DD-peptidases.

As employed herein, the term “pro-drug” refers to pharmacologically acceptable derivatives, e.g., esters and amides, such that the resulting biotransformation product of the derivative is the active drug. Pro-drugs are known in the art and are described generally in, e.g., Goodman and Gilman, “Biotransformation of Drugs”, In The Pharmacological Basis of Therapeutics, 8th Ed., McGraw Hill, Int. Ed. 1992, p. 13-15, which is hereby incorporated by reference in its entirety. Compounds of the invention may be formulated by any method well known in the art and may be prepared for administration by any route, including, without limitation, parenteral, oral, sublingual, transdermal, topical, intranasal, intratracheal, or intrarectal. In certain particularly preferred embodiments, compounds of the invention are administered intravenously in a hospital setting. In certain other embodiments, administration may be preferably by the oral route.

The invention also provides methods for inhibiting bacterial growth, such methods comprising administering to a bacterial cell culture, or to a bacterially infected cell culture, tissue, or organism, a β-lactamase inhibitor of Formula (I) or Formula (II) as defined for the first aspect of the invention.

Preferably, the bacteria to be inhibited by administration of a β-lactamase inhibitor of the invention are bacteria that are resistant to β-lactam antibiotics. More preferably, the bacteria to be inhibited are β-lactamase positive strains that are highly resistant to β-lactam antibiotics. The terms “slightly resistant” and “highly resistant” are well-understood by those of ordinary skill in the art (see, e.g., Payne et al., Antimicrobial Agents and Chemotherapy 38:767-772 (1994); Hanaki et al., Antimicrobial Agents and Chemotherapy 30:11.20-11.26 (1995)). Preferably, “highly resistant” bacterial strains are those against which the MIC of methicillin is >100 μg/mL. Preferably, “slightly resistant” bacterial strains are those against which the MIC of methicillin is >25 μg/mL.

The methods according to this aspect of the invention are useful for inhibiting bacterial growth in a variety of contexts. In certain preferred embodiments, the compound of the invention is administered to an experimental cell culture in vitro to prevent the growth of β-lactam resistant bacteria. In certain other preferred embodiments the compound of the invention is administered to an animal, including a human, to prevent the growth of β-lactam resistant bacteria in vivo. The method according to this embodiment of the invention comprises administering a therapeutically effective amount of β-lactamase inhibitor according to the invention for a therapeutically effective period of time to an animal, including a human. Preferably, the β-lactamase inhibitor is administered in the form of a pharmaceutical composition-according to the second aspect of the invention.

The terms “therapeutically effective amount” and “therapeutically effective period of time” are used to denote known treatments at dosages and for periods of time effective to show a meaningful patient benefit, i.e., healing of conditions associated with bacterial infection, and/or bacterial drug resistance. Preferably, such administration should be parenteral, oral, sublingual, transdermal, topical, intranasal, intratracheal, or intrarectal. When administered systemically, the therapeutic composition is preferably administered at a sufficient dosage to attain a blood level of inhibitor of at least about 100 micrograms/mL, more preferably about 1 milligram/mL, and still more preferably about 10 milligrams/mL. For localized administration, much lower concentrations than this may be effective, and much higher concentrations may be tolerated.

In certain preferred embodiments of the method according to this aspect of the invention, a β-lactamase inhibitor according to the invention is co-administered with an antibiotic. Preferably, such co-administration produces a synergistic effect. As employed herein, the terms “synergy” and “synergistic effect” indicate that the effect produced when two or more drugs are co-administered is greater than would be predicted based on the effect produced when the compounds are administered individually. While not wishing to be bound by theory, the present inventors believe that the β-lactamase inhibitors according to the invention act to prevent degradation of β-lactam antibiotics, thereby enhancing their efficacy and producing a synergistic effect. In particularly preferred embodiments of the invention, therefore, the co-administered antibiotic is a β-lactam antibiotic. For purposes of this invention, the term “co-administered” is used to denote simultaneous or sequential administration.

Synergy may be expressed as a ratio of the minimum inhibitory concentration (MIC) of an antibiotic tested in the absence of a β-lactamase inhibitor to the MIC of the same antibiotic tested in the presence of the β-lactamase inhibitor. A ratio of one (1) indicates that the β-lactamase inhibitor has no effect on antibiotic potency. A ratio greater than one (1) indicates that the β-lactamase inhibitor produces a synergistic effect when co-administered with the antibiotic agent. Preferably the β-lactamase inhibitor produces a synergy ratio of at least about 2, more preferably about 4, and still more preferably about 8. Most preferably, the β-lactamase inhibitor produces a synergy ratio of at least about 16. Alternatively, the synergy effect may be expressed as a factor, again, utilizing a concentration of the BLI to lower the MIC of the antibiotic. Thus, if the MIC of the antibiotic is 20 μg/mL and a 1.56 μM concentration of BLI lowers the MIC to 5 μg/mL, the synergy effect is four fold or “4X synergy”.

In certain other preferred embodiments, the β-lactamase inhibitor according to the invention may itself have antibiotic activity, and thus potentially can be administered alone or can be co-administered with a β-lactam antibiotic or any other type of antibiotic.

The term “antibiotic” is used herein to describe a compound or composition which decreases the viability of a microorganism, or which inhibits the growth or proliferation of a microorganism. “Inhibits the growth or proliferation” means increasing the generation time by at least 2-fold, preferably at least 10-fold, more preferably at least 100-fold, and most preferably indefinitely, as in total cell death. As used in this disclosure, an antibiotic is further intended to include an antimicrobial, bacteriostatic, or bactericidal agent. Non-limiting examples of antibiotics useful according to this aspect of the invention include penicillins, cephalosporins, aminoglycosides, sulfonamides, macrolides, tetracyclins, lincosides, quinolones, chloramphenicol, carbapenems, vancomycin, metronidazole, rifampin, isoniazid, spectinomycin, trimethoprim, sulfamethoxazole, and others. The term β-lactam antibiotic” is used to designate compounds with antibiotic properties containing a β-lactam functionality. Non-limiting examples of β-lactam antibiotics useful according to this aspect of the invention include penicillins, cephalosporins, penems, carbapenems, and monobactams.

Generally, the compounds of the invention can be routinely synthesized using techniques known to those skilled in the art (see U.S. Pat. No. 6,472,406, incorporated herein in its entirety) in conjunction with the teachings herein.

The following examples are intended to further illustrate certain preferred embodiments of the invention, and are not intended to limit the scope of the invention. In Schemes 1 through 4 the abbreviated terms are defined as: Boc=CO₂t-Bu, DMAP=4-dimethylaminopyridine, PMB=para-methoxybenzyl, TEA=triethylamine, DIAD=diisopropylazodicarboxylate, TFA=trifluoroacetic acid, TMSBr=bromotrimethylsilane, R=methyl or ethyl, R₁ is defined, HETAR=heteroaromatic/aromatic, L=linker, n=1-3, X=leaving group, Nuc=nucleophile (all as defined).

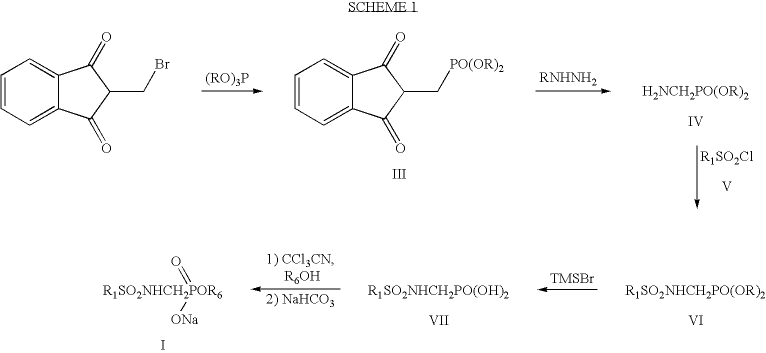

Generally, the compounds of the invention can be routinely synthesized using techniques known to those skilled in the art in conjunction with the teachings herein. The compounds of Formula (I) can be prepared in certain preferred embodiments according to the general synthetic route depicted in Scheme 1. Thus, Arbusov reaction of bromomethylphthalimide with a phosphite such as triethylphosphite is preferably conducted at elevated temperature, e.g., 145° C., in a solvent such as xylenes to afford the phthalimidomethylphosphonate III. Treatment of III with a hydrazine such as methylhydrazine in an alcoholic solvent such as methanol effects phthalimide cleavage to afford the aminomethylphosphonate IV. Treatment of IV with a sulfonyl chloride of the general formula V in an organic solvent such as methylene chloride, and in the presence of a base such as triethylamine, provides the N-sulfonylaminomethylphosphonate VI. Treatment of VI with a silyl halide such as trimethylsilyl bromide at room temperature in a solvent such as methylene chloride effects cleavage of the phosphonate ester to provide the phosphonic acid VII. In situ activation of VII with trichloroacetonitrile in pyridine, followed by treatment at 100° C. with an aryl or heteroaryl alcohol, such as phenol or substituted phenol, affords an aryl or heteroaryl phosphonate. Treatment with an aqueous base such as sodium bicarbonate then provides the sodium salt I, M=Na⁺, which corresponds to the compound of Formula (I).

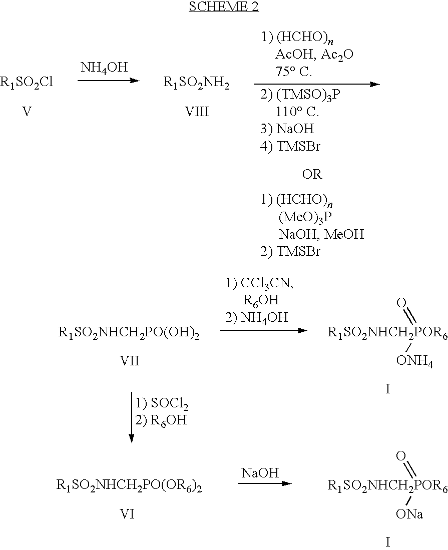

In certain other preferred embodiments, sulfonamidemethylphosphonates of formula I may be prepared according to the procedures illustrated in Scheme 2. Thus, sulfonyl chloride V is treated with ammonium hydroxide to produce the corresponding sulfonamide of formula VIII. Treatment of VIII with paraformaldehyde in the presence of a phosphite such as trimethylphosphite affords the phosphonate diester which in turn upon exposure to a silyl halide such as bromotrimethylsilane (TMSBr) produces the phosphonic acid of formula VII. Alternatively, the Arbusov reaction of VIII maybe performed with paraformaldehyde in the presence of acetic anhydride and a phosphite such as trimethylsilylphosphite to produce a more labile diester which as before readily yields VII. It is further noted, that this alternative often affects the formation of a small amount of N-acetylated by product which is easily hydrolyzed to the desired product by treatment sodium hydroxide during workup. The phosphonic acid VII may be converted to I by treatment with trichloroacetonitrile in pyridine in the presence of an aryl or heteroaryl alcohol, as described above, followed by basicification with ammonium hydroxide. Alternatively, treatment of VII with a chlorinating agent such as sulfuryl chloride or thionyl chloride, followed by treatment with an aryl or heteroaryl alcohol, affords the diester VI, which is mono-deprotected by treatment with base to afford I.

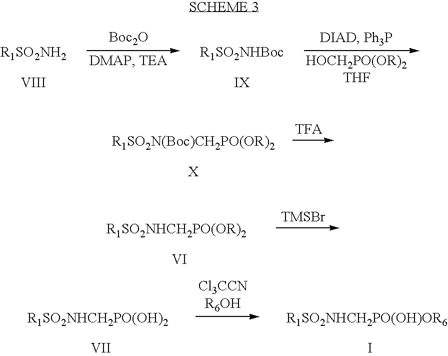

As an alternative to the synthetic process outlined in Scheme 2, sulfonamide VIII may be converted to the products of the invention by the route exhibited in Scheme 3. Thus, VIII is converted to the corresponding N-Boc-derivative IX by exposure to a t-butylcarbonylating agent such as t-butylchoroformate or di-t-butyl dicarbonate (Boc₂O) in the presence of a base such as triethylamine (TEA) and a catalytic amount of 4-N,N-dimethylaminopyridine (DMAP). Mitsunobu reaction of IX with either dimethyl or diethyl-hydroxymethylphosphonate in the presence of a phosphine or a phosphine based resin such as triphenylphosphine and a diazodicarboxylate such as diisopropylazodicarboxylate (DIAD) provides X. Removal of the Boc group of X is accomplished with trifluoroacetic acid to yield the phosphonate intermediate VI. As previously described, VI is converted to the products of the invention I.

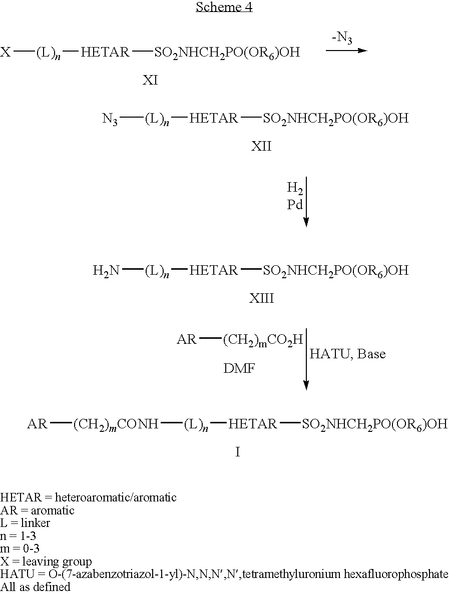

In certain other preferred embodiments, sulfonamidemethylphosphonates of formula I may be prepared according to the procedures illustrated in Scheme 4. Intermediate XI is converted to the azide intermediate XII, by displacement of the leaving group X with an azide salt, such as sodium or lithium azide or the like, in a suitable solvent such as DMSO, DMF, or DMA or the like. In turn XII is then converted to intermediate XIII, by reduction of the azide group with hydrogen gas in the presence of palladium black. The amine intermediate XIII is converted to the products of the invention I by reaction with an appropriate carboxylic acid in the presence of a suitable base, such as diisopropylethylamine or the like, and a coupling reagent, such as O-(7-azabenzotriazol-1-yl)-N,N,N′,N′,-tetramethyluronium hexafluorophosphate (HATU) or the like, in a solvent like DMF. Further, if the carboxylic acid contains protecting groups an additional deprotection step maybe warranted which is exemplified in the experimental section.

In certain other preferred embodiments, sulfonamidomethylphosphonates of Formula I are synthesized by more specific or less general chemistry which are exemplified in the experimental section.

### Preparative Example 1

### Preparation of 1-[(2-FLUORO-4-METHOXYPHENYL)THIO]ACETONE

To a stirred solution of 2-fluoro-4-methoxythiophenol (4.7 g, 29.7 mmol) in 150 mL of anhydrous acetone was added K₂CO₃ (8.2 g, 59.5 mmol) and chloroacetone (4.7 mL, 59.4 mmol). The resulting suspension was stirred at room temperature for 18 hrs. The mixture was filtered and the filtrate was concentrated in vacuo. The crude product was purified by silica gel chromatography (230-400 mesh) with 10% EtOAc-hexanes as the eluent to give of product as yellow oil.

¹H NMR (500 MHz, CDCl₃) δ (ppm): 7.39 (t, J=8.3 Hz, 1H), 6.7 (m, 2H), 3.81 (s, 3H), 3.54 (s, 2H), 2.30 (s, 3H).

### Preparative Example 2

### Preparation of 1-[(3,4-DIMETHOXYPHENYL)THIO]BUTAN-2-ONE

Following the foregoing procedure, this compound was synthesized by the reaction of 3,4-dimethoxybenzenethiol, 1-bromo-2-butanone and K₂CO₃ in acetone at room temperature. The reaction mixture was filtered and the filtrate then was partitioned between saturated NaHCO₃ and EtOAc, the layers were separated, and the aqueous layer was extracted again with EtOAc. The combined organic layers were dried (MgSO₄), filtered, and evaporated in vacuo. The resulting solid was purified by silica gel chromatography (EtOAc/Hexane=1:9) to provide the product.

¹H NMR (500 MHz, CDCl₃) δ (ppm): 7.00 (dd, 1H), 6.95 (d, 1H), 6.80 (d, 1H), 3.92 (d, 6H), 3.60 (s, 2H), 2.60 (q, 2H), 1.10 (t, 3H).

Utilizing the foregoing procedures, the following compound was prepared:

1-[(2,5-difluoro-3,4-dimethoxyphenyl)thio]butan-2-one

¹H NMR (500 MHz, CDCl₃) δ (ppm): 6.90 (m, 1H), 4.00 (d, 6H), 3.60 (s, 2H), 2.60 (q, 2H), 1.10 (t, 3H).

### Preparative Example 3

### Preparation of 4-METHOXY-2-NITRO-1-[(TRIFLUOROMETHYL)THIO]BENZENE

To a solution of commercially available 1-iodo-4-methoxy-2-nitrobenzene (1.75 g, 6.28 mmol) in anhydrous NMP (15 mL) was added trifluoromethylthiocopper (2.1 g, 12.6 mmol). The mixture was heated at 150° C. for 18 hrs, cooled to room temperature, and diluted with EtOAc. The organic phase was washed with cold 2N HCl, dilute aqueous NaHCO₃ solution and brine. The separated organic phase was dried with Na₂SO₄, filtered and evaporated. The resulting crude residue was purified by silica gel chromatography (230-400 mesh) with 10% EtOAc-hexanes as the eluent to give 4-methoxy-2-nitro-1-[(trifluoromethyl)thio]benzene.

¹H NMR (500 MHz, CDCl₃) δ (ppm): 7.42 (d, J=8.9 Hz, 1H), 7.47 (d, J=2.9 Hz, 1H), 7.18 (dd, J=8.9 Hz, 2.9 Hz, 1H), 3.93 (s, 2H).

### Preparative Example 4

### Preparation of 4-METHOXY-2-NITROPHENYL TRIFLUOROMETHYLSULFOXIDE

To a solution of 4-methoxy-2-nitro-1-[(trifluoromethyl)thio]benzene (1.05 g, 4.15 mmol) in dichloromethane [DCM] (30 mL) was added m-CPBA (1.43 g, 8.3 mmol, 75% technical grade). The mixture was stirred at room temperature for 18 hrs. The mixture was diluted with DCM and washed with 10% aq. Na₂S₂O₃, dilute NaHCO₃ solution and brine. The separated organic phase was dried with anhydrous Na₂SO₄, filtered and evaporated. The resulting residue was purified by silica gel chromatography (230-400 mesh) 25% using EtOAc-hexanes as the eluent to give 4-methoxy-2-nitrophenyl trifluoromethylsulfoxide.

¹H NMR (500 MHz, CDCl₃) δ (ppm): 8.24 (d, J=8.7 Hz, 1H), 7.89 (d, J=2.5 Hz, 1H), 7.51 (dd, J=8.9 Hz, 2.8 Hz, 1H), 4.03 (s, 3H).

### Preparative Example 5

### Preparation of 4-METHOXY-2-NITROPHENYL TRIFLUOROMETHYLSULFONE

To a stirred solution of chromium (VI) oxide (20.9 g, 209 mmol) in refluxing acetic acid (225 mL) was added carefully, drop wise over 30 min., a solution of 4-methoxy-2-nitrophenyl trifluoromethylsulfoxide (11.3 g, 41.9 mmol) in acetic acid (25 mL) (CAUTION: reaction may exotherm). The mixture was stirred at 110° C. for 18 hrs. The mixture was cooled to room temperature, diluted with EtOAc, and washed with water and brine. The organic layer was dried with Na₂SO₄, filtered and evaporated. The resulting residue was purified by silica gel chromatography (230-400 mesh) using 20% EtOAc-hexanes as the eluent to give 4-methoxy-2-nitrophenyl trifluoromethylsulfone.

¹H NMR (500 MHz, CDCl₃) δ (ppm): 8.12 (d, J=8.7 Hz, 1H), 7.33 (bs, 1H), 7.51 (d, J=9.0 Hz, 1H), 4.04 (s, 3H).

### Preparative Example 6

### Preparation of 2-FLUORO-4-METHOXYPHENYL TRIFLUOROMETHYLSULFONE

To a stirred solution of 4-methoxy-2-nitrophenyl trifluoromethylsulfone (4.5 g, 15.8 mmol) in anhydrous DMSO (200 mL) was added dry KF (1.83 g, 31.6 mmol) and tetraphenylphosphonium bromide (1.65 g, 3.94 mmol). The mixture was stirred at 130° C. for 25 min. The mixture was cooled to room temperature, diluted with EtOAc, and washed with water and brine. The organic layer was dried with Na₂SO₄, filtered and evaporated. The resulting residue was purified by silica gel chromatography (230-400 mesh) with 15% EtOAc-hexanes as the eluent to give 2-fluoro-4-methoxyphenyl trifluoromethylsulfone.

¹H NMR (500 MHz, CDCl₃) δ (ppm): 7.86 (t, J=8.1 Hz, 1H), 6.89 (dd, J=2.3 Hz, 1H), 6.78 (dd, J=11.7 Hz, 2.3 Hz, 1H), 3.93 (s, 3H).

### Preparative Example 7

### Preparation of 2-FLUORO-4-HYDROXYPHENYL TRIFLUOROMETHYLSULFONE

A mixture of 2-fluoro-4-methoxyphenyl trifluoromethylsulfone (1.95 g, 7.55 mmol) and pyridine hydrochloride (3.03 g, 26.2 mmol) was combined in a sealed tube and warmed to 160° C. for 3 hrs. The mixture was cooled to room temperature. The resulting solid was dissolved with water and EtOAc with sonication. The organic phase was washed with water and brine. The organic layer was dried with Na₂SO₄, filtered and evaporated. The resulting residue was purified by silica gel chromatography (230-400 mesh) with 30% EtOAc-hexanes as the eluent to give 2-fluoro-4-hydroxyphenyl trifluoromethylsulfone as a crystalline solid.

¹H NMR (500 MHz, CDCl₃) δ (ppm): 8.64 (s, 1H), 7.84 (t, J=8.4 Hz, 1H), 6.88 (dd, J=8.9 Hz, 2.3 Hz, 1H), 6.81 (dd, J=11.3 Hz, 2.3 Hz, 1H); ¹⁹F NMR (CDCl₃, ppm) −79.6 (ArSO₂CF₃), −101.9 (ArF).

### Preparative Example 8

### Preparation of 1-BROMO-3,4-DIMETHOXY-2-METHYLBENZENE

To a stirred solution of 1,2-dimethoxy-2-methoxy-3-methylbenzene (4 g, 26.3 mmol) in 35 mL THF at room temperature was added NBS (4.68 g, 26.3 mmol). The reaction mixture was stirred at room temperature overnight. The reaction mixture was partitioned between saturated NaHCO₃ and EtOAc, the layers were separated, and the aqueous layer was extracted again with EtOAc. The combined organic layers were dried (MgSO₄), filtered, and evaporated in vacuo. The resulting material was purified by silica gel chromatography (30% EtOAc in hexane) to provide the product.

¹H NMR (500 MHz, CDCl₃) δ (ppm): 7.28 (d, 1H), 6.70 (d, 1H), 3.90 (s, 3H), 3.86 (s, 3H), 2.40 (s, 3H).

Utilizing the foregoing procedure, the following compound was prepared:

1-bromo-4,5-dimethoxy-2-methylbenzene was prepared in 83% yield after purification by silica gel chromatography (30% EtOAc in hexane).

¹H NMR (500 MHz, CDCl₃) δ (ppm): 7.04 (s, 1H), 6.78 (s, 1H), 3.90 (s, 6H), 2.40 (s, 3H).

### Preparative Example 9

### Preparation of 3,4-DIMETHOXY-2-METHYLPHENYL BORONIC ACID

To a stirred solution of 1-bromo-3,4-dimethoxy-2-methoxy-3-methylbenzene (5 g, 21.6 mmol) in 50 mL THF at −78° C. was added 1.6N n-BuLi (16 mL, 26.0 mmol) drop wise. After addition, the solution was allowed to stir at −78° C. for 10 min. Then, triisopropylborate (6 mL, 26.0 mmol) was added and the mixture was stirred further for 30 min. 2N HCl (50 mL) was added to the reaction mixture and it was stirred at ambient temperature for 4 h. The reaction mixture was partitioned between saturated NaHCO₃ and EtOAc, the layers were separated, and the aqueous layer was extracted again with EtOAc. The combined organic layers were dried (MgSO₄), filtered, and evaporated in vacuo. The resulting material was purified by silica gel chromatography (40% EtOAc in hexane) to provide the product.

¹H NMR (500 MHz, CDCl₃) δ (ppm): 8.04 (d, 1H), 6.95 (d, 1H), 4.00 (s, 3H), 3.86 (s, 3H), 2.80 (s, 3H).

Utilizing the foregoing procedure, the following compound was prepared:

4,5-dimethoxy-2-methylphenyl boronic acid was prepared and purified by silica gel chromatography (40% EtOAc in hexane).

¹H NMR (500 MHz, CDCl₃) δ (ppm): 7.80 (s, 1H), 6.80 (s, 1H), 3.98 (d, 6H), 2.80 (s, 3H).

### Preparative Example 10

### Preparation of 5-(3,4-DIMETHOXYPHENYL)THIOPHENE-2-SULFONAMIDE

A stirred solution of 5-bromothiophene-2-sulfonamide (2 g, 8.26 mmol) and (3,4-dimethoxyphenyl)boronic acid (1.8 g, 9.90 mmol) in 30 mL DMF at room temperature was purged with N₂ gas for 10 mins. To this stirred solution was added (Ph₃P)₄Pd (1.9 g, 1.65 mmol) and saturated Na₂CO₃/H₂O (2.63 g, 3 mmol). The N₂ purge was stopped and the reaction mixture was sealed and heated with a preheated oil bath at 100° C. overnight. The reaction mixture was filtered and the filtrate was partitioned between saturated NaHCO₃ and EtOAc, the layers were separated, and the aqueous layer was extracted again with EtOAc. The combined organic layers were dried (MgSO₄), filtered, and evaporated in vacuo. The resulting material was purified by silica gel chromatography (40% EtOAc in hexane) to provide the product, as a light yellow solid.

¹H NMR (500 MHz, CD₃OD) δ (ppm): 7.56 (d, 1H), 7.30 (d, 1H), 7.24 (m, 2H), 7.00 (d, 1H), 3.90 (d, 6H).

Utilizing the foregoing procedure, the following compounds were prepared:

5-(3,4-dimethoxy-2-methylphenyl)thiophene-2-sulfonamide was prepared and purified by silica gel chromatography (40% EtOAc in hexane) to provide the product, as a white solid.

¹H NMR (500 MHz, CD₃OD) δ (ppm): 7.60 (d, 1H), 7.16 (d, 1H), 7.00 (d, 2H), 7.00 (d, 1H), 6.96 (d, 1H), 3.90 (d, 3H), 3.80 (s, 3H), 2.30 (s, 3H).

5-(4,5-dimethoxy-2-methylphenyl)thiophene-2-sulfonamide was prepared and purified by silica gel chromatography (40% EtOAc in hexane).

¹H NMR (500 MHz, CD₃OD) δ (ppm): 7.58 (d, 1H), 7.06 (d, 1H), 6.94 (s, 1H), 6.90 (s, 1H), 3.82 (d, 6H), 2.40 (s, 3H).

### Preparative Example 11

### Preparation of 5-(4,5-DIHYDROXY-2-METHYLPHENYL)THIOPHENE-2-SULFONAMIDE

To a stirred solution of thiophene derivative (1.22 g, 3.9 mmol) in dichloromethane was added drop wise BBr₃ (8 mL, 2 equiv.). The resulting mixture was stirred under nitrogen for 1 h. The reaction mixture was partitioned between saturated NaHCO₃ and EtOAc. The organic layer was washed with water and brine, dried over magnesium sulfate and concentrated. The crude product was used as is in the next step.

¹H NMR (500 MHz, CD₃OD) δ (ppm): 7.60 (d, 1H), 6.96 (d, 1H), 6.82 (s, 1H), 6.70 (d, 1H), 2.26 (s, 3H).

Utilizing the foregoing procedure the following compound was prepared: 5-(3,4-dihydroxy-2-methylphenyl)thiophene-2-sulfonamide was prepared and used as is for next step.

¹H NMR (500 MHz, CD₃OD) δ (ppm): 7.60 (d, 1H), 6.96 (d, 1H), 6.80 (s, 1H), 6.70 (d, 1H), 2.24 (s, 3H).

### Preparative Example 12

### N-[(DIMETHYLAMINO)METHYLENE]-(3,4-DIHYDROXYPHENYL)-THIOPHENE-2-SULFONAMIDE

A solution of the sulfonamide derivative (1.8 g, 6.9 mmol) and DMF-dimethylacetal (1 mL, 1.1 equiv) in anhydrous DMF (15 mL) was stirred at room temperature for 2 h. The reaction mixture was diluted with EtOAc and washed twice with water. The organic extract was dried with magnesium sulfate and concentrated. The crude product was used as is in the next step.

¹H NMR (500 MHz, CD₃OD) δ (ppm): 8.20 (s, 1H), 7.44 (d, 1H), 7.32 (d, 1H), 7.08 (s, 1H), 7.00 (dd, 1H), 6.80 (d, 1H), 3.20 (s, 3H), 2.82 (s, 3H).

Utilizing this procedure, the following compounds were prepared:

N-[(dimethylamino)methylene]-5-(3,4-dihydroxy-2-methylphenyl)thiophene-2-sulfonamide was prepared and used as is for next step.

¹H NMR (500 MHz, CD₃OD) δ (ppm): 8.20 (s, 1H), 7.50 (d, 1H), 6.92 (d, 1H), 6.72 (d, 1H), 6.70 (d, 1H), 3.20 (s, 3H), 3.10 (s, 3H), 2.22 (s, 3H).

N-[(dimethylamino)methylene]-5-(4,5-dihydroxy-2-methylphenyl)thiophene-2-sulfonamide was prepared and used as is in the next step.

¹H NMR (500 MHz, CD₃OD) δ (ppm): 8.20 (s, 1H), 7.50 (d, 1H), 6.96 (d, 1H), 6.82 (s, 1H), 6.70 (s, 1H), 3.20 (s, 3H), 3.06 (s, 3H), 2.22 (s, 3H); MS m/z 341 (M+1).

### Preparative Example 13

### Preparation of 5-[3,4-BIS(3-CHLOROPROPOXY)PHENYL]-N-[(1E)-(DIMETHYLAMINO)-METHYLENE]THIOPHENE-2-SULFONAMIDE

A mixture of the product of the prior example (2.2 g, 6.9 mmol), Cs₂CO₃ (8.7 g, 4 equiv), and 1,3-bromochloropropane (6.6 mL, 10 equiv) in DMF was stirred at room temperature for 20 h. The white inorganic solid was filtered off and washed with EtOAc. The filtrate was washed with water, and dried over magnesium sulfate. Removal of the solvent gave the crude product which was used as is in the next step.

¹H NMR (500 MHz, CD₃OD) δ (ppm): 8.20 (s, 1H), 7.50 (d, Hi), 7.24 (m, 3H), 7.04 (d, 1H), 4.20 (m, 4H), 3.80 (m, 4H), 3.20 (s, 3H), 3.04 (s, 3H), 2.26 (m, 4H); MS m/z 479 (M).

Utilizing the foregoing procedure, the following compounds were prepared:

5-[3,4-bis(3-chloropropoxy)-2-methylphenyl]-N-[(1E)-(dimethylamino)methylene]thiophene-2-sulfonamide was prepared and used as is for next step.

¹H NMR (500 MHz, CD₃OD) δ (ppm): 8.20 (s, 1H), 7.50 (d, 1H), 7.10 (d, 1H), 7.00 (d, 1H), 6.98 (d, 1H), 4.20 (t, 2H), 4.10 (t, 2H), 3.84 (m, 4H), 3.20 (s, 3H), 3.04 (s, 3H), 2.26 (m, 7H).

5-[4,5-bis(3-chloropropoxy)-2-methylphenyl]-N-[(1E)-(dimethylamino)methylene]thiophene-2-sulfonamide was prepared and used as is for next step.

¹H NMR (500 MHz, CD₃OD) δ (ppm): 8.20 (s, 1H), 7.52 (d, 1H), 7.06 (d, 1H), 7.00 (s, 1H), 6.98 (s, 1H), 4.22 (t, 2H), 4.14 (t, 2H), 3.80 (m, 4H), 3.36 (s, 6H), 2.26 (m, 7H).

### Preparative Example 14

### Preparation of 5-[3,4-BIS(3-CHLOROPROPOXY)PHENYL]THIOPHENE-2-SULFONAMIDE

To a stirred solution of the crude formamidine, prepared in the prior example, in acetone was added excess NH₄OH (1:1) at room temperature. The reaction mixture was stirred for 2 days and the volatiles were evaporated. The residue was dissolved in EtOAc and the organic layer was washed with water, dried and concentrated. The crude product was used as is in next step.

¹H NMR (500 MHz, CD₃OD) δ (ppm): 7.68 (d, 1H), 7.08 (d, 1H), 7.04 (m, 2H), 7.00 (d, 1H), 4.20 (m, 4H), 3.80 (m, 4H), 2.22 (m, 4H); MS m/z 424 (M).

Utilizing the foregoing procedure, the following compounds were prepared:

5-[3,4-bis(3-chloropropoxy)phenyl]thiophene-2-sulfonamide was prepared and used as is for next step.

¹H NMR (500 MHz, CD₃OD) δ (ppm): 7.68 (d, 1H), 7.08 (d, 1H), 7.04 (m, 2H), 7.00 (d, 1H), 4.20 (m, 4H), 3.80 (m, 4H), 2.22 (m, 4H); MS m/z 424 (M⁺).

5-[3,4-bis(3-chloropropoxy)-2-methylphenyl]thiophene-2-sulfonamide was prepared and used as is for next step. ¹H NMR (500 MHz, CD₃OD) δ (ppm): 7.58 (d, 1H), 7.10 (d, 1H), 6.98 (d, 1H), 6.94 (d, 1H), 4.20 (t, 2H), 4.10 (t, 2H), 3.80 (m, 4H), 2.22 (m, 4H), 2.20 (s, 3H).
5-[4,5-bis(3-chloropropoxy)-2-methylphenyl]thiophene-2-sulfonamide was prepared and used as is for next step.

¹H NMR (500 MHz, CD₃OD) δ (ppm): 7.58 (d, 1H), 7.00 (d, 1H), 6.98 (s, 1H), 6.92 (s, 1H), 4.20 (t, 2H), 4.10 (t, 2H), 3.80 (m, 4H), 2.34 (s, 3H), 2.22 (m, 4H).

### Preparative Example 15

### 3,4-BIS(3-CHLOROPROPOXY)BENZENAMINE

To a stirred solution of 4-nitrobenzene-1,2-diol (14.1 g, 91.0 mmol) in acetone (200 mL) at room temperature was successively added 1-bromo-3-chloropropane (26.86 mL, 273 mmol), K₂CO₃ (37.73 g, 273 mmol) and KI (3.02 g, 18.2 mmol). The reaction mixture was heated to reflux overnight under N₂ and then cooled to rt. The reaction mixture was filtered and the filtrate was concentrated. The residue was dissolved in EtOAc (300 mL) and then the resulting solution was washed with water (80 mL×2) and brine (150 mL). The organic phase was dried over Na₂SO₄ and filtered. The filtrate was concentrated and the residue was purified by flash column chromatography (eluent EtOAc/hexanes: from 10:90 to 20:80) to produce an amorphous brownish material.

MS m/z 308 (M+1).

This material was dissolved in AcOH (150 mL) and iron powder (23.4 g, 428.3 mmol) was added. The resulting reaction mixture was stirred at rt overnight under N₂ and filtered through a celite pad. The filtrate was concentrated, the residue was diluted with water (100 mL) and made alkaline with a solution of NaOH (50%) to pH=10. The resulting basic solution was extracted with EtOAc (200 mL×3); the combined extracts were washed with water (80 mL) and brine (150 mL). The organic phase was dried over Na₂SO₄ and then filtered. The filtrate was concentrated to dryness and the residue was purified by flash column chromatography (eluent EtOAc/hexanes, 30:70) to afford the title compound as pale brown oil. MS m/z 278 (M+1).

### Preparative Example 16

### DIMETHYL 2,2′-(4-NITRO-1,2-PHENYLENE)BIS(OXY)DIACETATE

To a stirred suspension of 4-nitrocatechol (1.10 g, 7.09 mmol) and potassium carbonate (4.0 g, 29 mmol) in acetone (100 mL) was added methyl bromoacetate (1.3 mL, 2.1 g, 14 mmol,). The mixture was heated to reflux for 3 h, then cooled and concentrated under reduced pressure. The residue was partitioned between water and ethyl acetate, and the combined organic extracts were washed with aqueous sodium hydroxide (1 M), saturated ammonium chloride, and brine, dried over magnesium sulfate, and concentrated. The residue was triturated with 1:1 ethyl acetate/hexanes to provide the product as a colorless solid.

¹H NMR (400 MHz, DMSO-d₆) δ (ppm): 7.88 (dd, J=9.0, 2.7, 1H); 7.74 (d, J=2.7, 1H); 7.14 (d, J=9.0, 1H); 5.02 (s, 2H); 5.00 (s, 2H); 3.70 (s, 3H); 3.69 (s, 3H).

### Preparative Example 17

### 3,4-BIS(2-CHLOROETHOXY)ANILINE

To a stirred suspension of the product from the prior example (7.68 g, 25.7 mmol,) and sodium borohydride (1.15 g, 30.4 mmol) in THF (300 mL) was added methanol (5.0 mL) and the resulting mixture was stirred for 2 h. It was then concentrated, partitioned between ethyl acetate and water, and the combined organic extracts were washed with water, brine, dried over magnesium sulfate, and concentrated. The resulting crude diol was suspended in toluene (100 mL) and pyridine (5 mL), and heated to reflux. Thionyl chloride (5 mL) was then added dropwise, and the suspension was heated under reflux for 1 hour. The toluene was then removed under reduced pressure, the residue was partitioned between ether and water, and the combined organic extracts were washed with water, dried over magnesium sulfate, and concentrated. To this crude dichloride in methanol (200 mL) was added palladium on activated carbon (10%, 0.50 g), and this was stirred under an atmosphere of H₂ for 6 h. The mixture was then filtered through celite, concentrated under reduced pressure, and purified by column chromatography (55% ethyl acetate/hexanes) to yield the product as an off-white, crystalline solid.

¹H NMR (400 MHz, DMSO-d₆) δ (ppm): 6.69 (d, J=8.4, 1H); 6.25 (d, J=2.5, 1H); 6.08 (dd, J=8.4, 2.5, 1H); 4.80 (br s, 2H); 4.14-4.11 (m, 2H), 4.07-4.04 (m, 2H); 3.91-3.87 (m, 2H); 3.81-3.77 (m, 2H);

### Preparative Example 18

### 5,6-BIS(2-CHLOROETHOXY)BENZO[d]THIAZOL-2-AMINE

To a suspension of lead thiocyanate (6.6 g, 20.4 mmol) in acetic acid (40 mL) was added bromine (1.2 mL, 3.7 g, 23.4 mmol) dropwise, and the mixture was stirred until the orange color has almost completely faded (10 min). This solution was then filtered into the product of the prior example (3.35 g, 13.5 mmol) in acetic acid (30 mL); the mixture was stirred for 30 min, and then concentrated under reduced pressure. The solid was suspended in ethyl acetate and filtered over suction. The resulting solid was suspended in saturated sodium bicarbonate and extracted repeatedly with ethyl acetate. The combined organic extracts were washed with brine, dried over magnesium sulfate, and concentrated. Column chromatography (ethyl acetate) afforded the product. MS m/z 307 (M).

Utilizing the foregoing procedure, the following compound was prepared:

5,6-Bis(3-chloropropoxy)benzo[d]thiazol-2-amine. Prepared 6.5 g (100% yield), as a pale yellow solid.

MS m/z 335 (M+1).

### Preparative Example 19

### 5,6-BIS(2-CHLOROETHOXY)BENZO[d]THIAZOLE

5,6-Bis(2-chloroethoxy)benzo[d]thiazol-2-amine (2.00 g, 6.51 mmol) was stirred in 85% phosphoric acid (50 mL) overnight to effect a partial solution. It was then cooled to 0° C., and sodium nitrite (0.60 g, 8.7 mmol) in water (5 mL) was added, and the mixture was stirred for 10 min. This was then poured into hypophosphorous acid (100 mL) at r.t. and left to stand for 18 h. It was then diluted with water and extracted multiple times with ethyl acetate. The combined organic layers were filtered through a short plug of silica (to remove insoluble solids) and concentrated, and the resulting solid was triturated with 1:1 ethyl acetate/hexanes to provide the product as a solid.

¹H NMR (400 MHz, CD₃OD) δ (ppm): 9.14 (s, 1H); 7.67 (s, 1H); 7.63 (s, 1H); 4.39-4.34 (m, 4H); 3.94-3.90 (m, 4H).

Utilizing the foregoing procedure, the following compound was prepared:

5,6-bis(3-chloropropoxy)benzo[d]thiazole was purified by flash chromatography on silica gel using 5% EtOAc/DCM as an eluent, followed by trituration with diethyl ether to afford product as a pale yellow solid.

MS m/z 320 (M+1).

### Preparative Example 20

### 5,6-BIS(2-CHLOROETHOXY)BENZO[d]THIAZOLE-2-THIOL

To 5,6-bis(2-chloroethoxy)benzo[d]thiazole (2.4 g, 8.2 mmol) in THF (100 mL) was added n-butyllithium (2.5 M in hexanes, 4.1 mL, 10.3 mmol) dropwise, at −78° C. The mixture was stirred for 30 min, then sulfur (1.25 g, 39.0 mmol) suspended in THF (5 mL) was added, and the mixture was stirred for a further 30 min at −78° C. The reaction was quenched with ˜1 mL saturated ammonium chloride and concentrated under reduced pressure. The residue was partitioned between aqueous sodium hydroxide (1M) and ethyl acetate, and the combined aqueous layers were acidified with hydrochloric acid (1M), extracted with ethyl acetate, washed with aqueous ammonium chloride, dried over magnesium sulfate, and concentrated. The crude product was triturated with 1:1 dichloromethane/hexanes to provide the product as a colorless solid.

¹H NMR (400 MHz, DMSO-d₆) δ (ppm): 13.59 (br s, 1H); 7.42 (s, 1H); 6.88 (s, 1H); 4.27-4.21 (m, 4H); 4.00-3.90 (m, 4H).

Utilizing the foregoing procedure, the following compound was prepared:

5,6-bis(3-chloropropoxy)benzo[d]thiazole-2-thiol was purified by flash column chromatography using 40% EtOAc/DCM as an eluent to produce product as a pale yellow solid.

¹H NMR (400 MHz, DMSO-d₆) [symbol] ppm): 13.60 (bs, 1H), 7.40 (s, 1H), 6.86 (s, 1H), 4.06 (q, J=5.9 Hz, 4H), 3.78 (q, J=6.5 Hz, 4H), 2.16 (m, 4H); MS m/z 350 (M−1]).

### Preparative Example 21

### DIETHYL (5,6-BIS(2-CHLOROETHOXY)BENZO[d]THIAZOL-2-YLTHIOAMINO)-METHYLPHOSPHONATE

To 5,6-bis(2-chloroethoxy)benzo[d]thiazole-2-thiol (1.10 g, 3.39 mmol) in DME (10 mL) was added aqueous sodium hydroxide (1.25 M, 5.4 mL, 6.8 mmol) and the mixture was diluted to 25 mL with water. Sodium hypochlorite (12% solution, 2.4 mL, 0.29 g, 3.9 mmol) was diluted with water (25 mL). These two solutions were added simultaneously, dropwise, to a stirred solution of diethyl aminomethylphosphonate oxalate (2.10 g, 8.17 mmol) and aqueous sodium hydroxide (1.25 M, 6.6 mL, 8.2 mmol) in DME (10 mL), at 0° C., over ˜10 min. The mixture was stirred for 10 min, and then the DME was removed under reduced pressure. The residue was partitioned between water and ethyl acetate, and the combined organic extracts were washed with saturated ammonium chloride and brine, dried over magnesium sulfate, and concentrated. Column chromatography (ethyl acetate) provided the product.

¹H NMR (400 MHz, CD₃OD) δ (ppm): 7.56 (s, 1H); 7.38 (s, 1H); 4.35-4.31 (m, 4H); 4.19 (quint, J=6.9, 4H); 3.92-3.87 (m, 4H); 3.56 (d, J=10.4, 2H); 1.35 (t, J=7.0, 6H).

Utilizing the foregoing procedure, the following compound was prepared:

diethyl (5,6-bis(3-chloropropoxy)benzo[d]thiazol-2-ylthioamino)methylphosphonate was purified by flash chromatography on silica gel (eluent 75% EtOAc/hexanes to 100% EtOAc) to give product, as a pale brownish yellow oil.

MS m/z 517 (M+1).

### Preparative Example 22

### General Preparation of Benzothiophenes

### Preparation of 7-CHLORO-5-METHOXY-1-BENZOTHIOPHENE

A mixture of 2-chloro-1-[(2,2-diethoxyethyl)thio]-4-methoxybenzene (22.7 g, 78.2 mmol) and PPA (100.8 g) in anhydrous toluene (250 mL) was vigorously stirred at reflux (110° C.) for 45 min. The mixture was cooled to 70° C. and water (200 mL) was carefully added over 10 min. After 2 hrs, the mixture was diluted with EtOAc and washed with water and brine. The organic layer was dried with Na₂SO₄, filtered and evaporated. The resulting residue was purified by silica gel chromatography (230-400 mesh) using 5% EtOAc-hexanes as the eluent to give 7-chloro-5-methoxy-1-benzothiophene.

¹H NMR (500 MHz, CDCl₃) δ (ppm): 7.52 (d, J=5.5 Hz, 1H), 7.32 (d, J=5.5 Hz, 1H), 7.24 (d, J=2.3 Hz, 1H), 7.08 (d, J=2.3 Hz, 1H), 3.91 (s, 3H).

Utilizing the foregoing procedure, the following compounds were prepared:

3-ethyl-5,6-dimethoxy-1-benzothiophene was purified by silica gel chromatography (EtOAc/Hexane=1:9).

¹H NMR (500 MHz, CDCl₃) δ (ppm): 7.30 (s, 1H), 7.20 (s, 1H), 7.00 (s, 1H), 4.00 (d, 6H), 2.85 (q, 2H), 1.40 (t, 3H).

4,7-difluoro-5,6-dimethoxy-1-benzothiophene

¹H NMR (400 MHz, CDCl₃) δ (ppm): 7.4 (m, 2H), 4.05 (s, 3H), 4.01 (s, 3H); MS m/z 231 (M+1).

4,7-difluoro-5,6-dimethoxy-3-ethyl-1-benzothiophene was purified by silica gel chromatography (EtOAc/Hexane=1:9).

¹H NMR (500 MHz, CDCl₃) δ (ppm): 7.00 (s, 1H), 4.05 (d, 6H), 2.95 (q, 2H), 1.36 (t, 3H).

### Preparative Example 23

### General Preparation of Benzothiophenes

### Method A. 5-HYDROXY-1-BENZOTHIOPHENE

5-Methoxy-1-benzothiophene (6.37 g, 38.8 mmol) was combined with pyridine hydrochloride (13.5 g, 116.4 mmol) in a sealed tube and heated to 190° C. for 3.5 hrs. The reaction was cooled to room temperature and the resulting residue was dissolved in 2:1 EtOAc-H₂O with sonication. The organic layer was washed with water, brine, dried over anhydrous Na₂SO₄, and filtered. The filtrate was evaporated in vacuo. The crude residue was purified by silica gel chromatography using 20% EtOAc-hexanes to give 5-hydroxy-1-benzothiophene.

¹H NMR (500 MHz, acetone-4) [symbol] ppm): 8.32 (s, 1H), 7.43 (d, J=8.8 Hz, 1H), 7.56 (d, J=5.5 Hz, 1H), 7.28 (d, J=2.3 Hz, 1H), 7.25 (d, J=5.3 Hz, 1H), 6.96 (dd, J=2.3 Hz, 1H).

Utilizing the foregoing procedure, the following compounds were prepared:

1-benzothiophene-5,6-diol was prepared in quantitative yield from 5,6-dimethoxy-1-benzothiophene; solidified in ether/hexane.

¹H NMR (400 MHz, CDCl₃) δ (ppm): 7.3 (s, 1H), 7.25 (s, 1H), 7.2 (d, 1H), 7.15 (d, 1H); MS m/z 167 (M+1).

3-ethyl-1-benzothiophene-5,6-diol was purified by silica gel chromatography (EtOAc/Hexane=2/3).

¹H NMR (500 MHz, CD₃OD) δ (ppm): 7.20 (s, 1H), 7.10 (s, 1H), 6.90 (s, 1H), 2.75 (q, 2H), 1.35 (t, 3H).

4,7-difluoro-3-ethyl-1-benzothiophene-5,6-diol

¹H NMR (500 MHz, CDCl₃) δ (ppm): 6.85 (s, 1H), 2.95 (q, 2H), 1.30 (t, 3H); MS m/z 231 (M+1).

### Method B. 4,7-DIFLUORO-5,6-DIHYDROXY-1-BENZOTHIOPHENE

To a stirred solution of the difluorobenzothiophene derivative (4 g, 17.39 mmol) in dichloromethane at −78° C. under nitrogen was added drop wise BBr₃ (41 mL, 2.2 equiv). The resulting mixture was immediately warmed up to room temperature and left overnight, after which time, it was quenched with MeOH. The mixture was washed with water, dried over magnesium sulfate, and concentrated to afford the crude product.

¹H NMR (400 MHz, CD₃OD) δ (ppm): 7.32 (d, 1H), 7.26 (dd, 1H); MS m/z 203 (M+1).

Utilizing the foregoing procedure, the following compounds were prepared:

5-(3,4-dihydroxyphenyl)thiophene-2-sulfonamide was prepared and used as is in the next step.

¹H NMR (500 MHz, CD₃OD) δ (ppm): 7.52 (d, 1H), 7.16 (d, 1H), 7.10 (s, 1H), 7.00 (dd, 1H), 6.80 (d, 1H).

5-(3,4-dihydroxy-2-methyl-phenyl)thiophene-2-sulfonamide was prepared and used as is in the next step.

¹H NMR (500 MHz, CD₃OD) δ (ppm): 7.60 (d, 1H), 6.96 (d, 1H), 6.80 (s, 1H), 6.70 (d, 1H), 2.24 (s, 3H).

### Preparative Example 24

### DIMETHYL 2,2′-[1-BENZOTHIENE-5,6-DIYLBIS(OXY)]DIACETATE

To a stirred solution of 3.9 g, (23.5 mmol) of 1-benzothiophene-5,6-diol in acetone was added sequentially K₂CO₃ (13 g, 4 equiv) and then 2-bromomethylacetate (8.9 mL, 4 equiv) and the reaction mixture was heated to reflux over a period of 5 h. The white inorganic solid was filtered off and washed with EtOAc. Removal of the solvents and subsequent chromatography gave the product as an oil.

¹H NMR (400 MHz, CDCl₃) δ (ppm): 7.4 (s, 1H), 7.38 (d, 1H), 7.33 (s, 1H), 7.24 (d, 1H), 4.56 (s, 4H), 3.75 (s, 6H); MS m/z 333 (M+23), 311 (M+1).

Utilizing the foregoing procedure, the following compound was prepared:

dimethyl 2,2′-[(4,7-difluoro-1-benzothiene-5,6-diyl)bis(oxy)]diacetate

¹H NMR (400 MHz, CDCl₃) δ (ppm): 7.42 (d, 1H), 7.38 (dd, 1H), 4.89 (s, 2H), 4.85 (s, 2H), 3.8 (s, 6H);

MS m/z 369 (M+23).

dimethyl 2,2′-[(3-methyl-1-benzothiene-5,6-diyl)bis(oxy)]diacetate

¹H NMR (500 MHz, CDCl₃) δ (ppm): 7.34 (s, 1H), 7.19 (s, 1H), 6.99 (s, 1H), 4.81 (d, 4H), 3.82 (d, 6H), 2.39 (s, 3H); MS m/z 325 (M+1).

dimethyl 2,2′-[(3-methyl-4,7-difluoro-1-benzothiene-5,6-diyl)bis(oxy)]diacetate

¹H NMR (500 MHz, CDCl₃) δ (ppm): 6.96 (s, 1H), 4.87 (s, 2H), 4.82 (s, 2H), 3.82 (2s, 6H), 2.52 (s, 3H).

dimethyl 2,2′-[(3-ethyl-4,7-difluoro-1-benzothiene-5,6-diyl)bis(oxy)]diacetate

Purified by silica gel chromatography (EtOAc/Hexane=2/3).

¹H NMR (500 MHz, CDCl₃) δ (ppm): 7.00 (s, 1H), 4.88 (d, 4H), 3.82 (d, 6H), 3.00 (q, 2H), 2.28 (m, 4H), 1.35 (t, 3H); MS m/z 375 (M+1).

### Preparative Example 25

### 2,2′-[1-BENZOTHIENE-5,6-DIYLBIS(OXY)]DIETHANOL

The bis-ester (6 g, 19.2 mmol), from the prior example, was reduced with NaBH₄ (7.3 g, 10 equiv) in 10% MeOH in THF at room temperature. After 1.5 h the reaction mixture was quenched with water/dilute HCL solution. The aqueous phase was partitioned with ethyl acetate, and subsequent washing of the combined organic layer with brine and concentration provided crude product.

¹H NMR (400 MHz, CDCl₃) δ (ppm): 7.5 (s, 1H), 7.41 (s, 1H), 7.39 (d, 1H), 7.24 (d, 1H), 4.2 (brm, 4H), 4.0 (brm, 4H); MS m/z 277 (M+1).

Utilizing the foregoing procedure, the following compounds were prepared:

2,2′-[(4,7-difluoro-1-benzothiene-5,6-diyl)bis(oxy)diethanol was prepared in 75% yield.

¹H NMR (400 MHz, CDCl₃) δ (ppm): 7.46 (d, 1H), 7.43 (dd, 1H), 4.36 (m, 4H), 3.96 (m, 4H); MS m/z 291 (M+1).

2,2′-[(3-methyl-1-benzothiene-5,6-diyl)bis(oxy)diethanol

¹H NMR (500 MHz, CD₃OD) δ (ppm): 7.42 (s, 1H), 7.24 (s, 1H), 7.01 (s, 1H), 4.13 (m, 4H), 3.92 (m, 4H), 2.38 (s, 3H).

2,2′-[(3-methyl-4,7-difluoro-1-benzothiene-5,6-diyl)bis(oxy)diethanol

Purification was accomplished by chromatography (Biotage Horizon system; 25 M column, 0% to 100% EtOAc/hexane over 10 column volume and 100% EtOAc for 2 column volume; flow rate=25 mL/min).

¹H NMR (500 MHz, CDCl₃) δ (ppm): 6.99 (s, 1H), 4.43 (2t, 4H), 3.88 (m, 4H), 2.59 (s, 3H).

2,2′-[(3-ethyl-4,7-difluoro-1-benzothiene-5,6-diyl)bis(oxy)diethanol

¹H NMR (500 MHz, CDCl₃) δ (ppm): 7.20 (s, 1H), 4.30 (t, 2H), 4.25 (t, 2H), 3.90 (m, 4H), 2.98 (q, 2H), 1.35 (t, 3H); MS m/z 319 (M+1).

### Preparative Example 26

### Method A. 5,6-BIS(2-CHLOROETHOXY)-1-BENZOTHIOPHENE

A solution of the crude diol (3 g, 12.9 mmol) and pyridine (3.3 mL, 3 equiv) in absolute benzene (50 mL) was stirred at 80° C. Thionyl chloride (2.4 mL, 3 equiv) was added to the solution drop wise over 1 h and then left for another 1.5 h. The mixture was cooled to room temperature and saturated copper sulfate solution added. The resulting organic phase was separated, washed twice with water, dried over magnesium sulfate, filtered, and evaporated to give an oily residue, which was purified by silica gel chromatography to afford the corresponding dichloride.

¹H NMR (400 MHz, CDCl₃) δ (ppm): 7.44 (s, 1H), 7.38 (s, 1H), 7.36 (d, 1H), 7.23 (d, 1H), 4.35 (t, 4H), 3.9 (t, 4H); MS m/z 291 (M+1).

Utilizing the foregoing procedure, the following compound was prepared:

5,6-bis(2-chloroethoxy)-4,7-difluoro-1-benzothiophene

¹H NMR (400 MHz, CDCl₃) δ (ppm) 7.45 (d, 1H), 7.42 (d, 1H), 4.45 (m, 4H), 3.8 (m, 4H); MS m/z 328 (M+1).

### Method B. 5,6-BIS(2-CHLOROETHOXY)-3-METHYL-1-BENZOTHIOPHENE

To a mixture of the product obtained from Preparative Example 25 (1.9810 g, 7.5 mmol) in CCl₄/distilled THF (120 mL/50 mL) was added polymer-supported triphenylphosphine (Aldrich, 12.67 g, 37 mmol). The reaction was heated to 80° C. under nitrogen and refluxed for 19 h. The resin was removed by filtration and the filtrate was concentrated in vacuo. The resulting residue was purified by chromatography using the Isco CombiFlash Companion (40 g column; 0% to 100% EtOAc/hexane over 15 min.; flow rate=40 mL/min; desired product elutes at 30% EtOAc/hexane) to give the product as a pale yellow solid.

¹H NMR (500 MHz, CDCl₃) δ (ppm): 7.00 (s, 1H), 7.24 (s, 1H), 7.40 (s, 1H), 4.33-4.39 (m, 4H), 3.90 (m, 4H), 2.41 (s, 3H).

Utilizing the foregoing procedure, the following compounds were prepared:

5,6-bis(2-chloroethoxy)-3-methyl-4,7-difluoro-1-benzothiophene

A pale yellow oil after purification by chromatography (Biotage Horizon system; 25 M column, 0% to 70% EtOAc/hexane over 10 column volume; flow rate=25 mL/min).

¹H NMR (500 MHz, CDCl₃) δ (ppm): 6.95 (s, 1H), 4.38-4.44 (2t, 4H), 3.85 (m, 4H), 2.54 (s, 3H).

5,6-bis(2-chloroethoxy)-3-ethyl-4,7-difluoro-1-benzothiophene

Purified by silica gel chromatography (EtOAc/Hexane=1/9).

¹H NMR (500 MHz, CD₃OD) δ (ppm): 7.20 (s, 1H), 4.45 (t, 2H), 4.40 (t, 2H), 3.90 (m, 4H), 3.00 (q, 2H), 1.35 (t, 3H).

### Preparative Example 27

### Preparation of 5,6-BIS(3-CHLOROPROPOXY)-1-BENZOTHIOPHENE

To a solution of 1-benzothiophene-5,6-diol (3 g, 18.1 mmol) in 35 mL DMF at room temperature was added Cs₂CO₃ (23 g, 72.4 mmol) and 1-bromo-3-chloropropane (36 mL, 362 mmol). The reaction was allowed to stir at room temperature for 2 h and monitored by TLC. The reaction mixture was partitioned between H₂O and EtOAc, the layers were separated, and the aqueous layer was extracted again with EtOAc. The combined organic layers were washed by H₂O, brine and dried (MgSO₄), filtered, and evaporated in vacuo. The resulting product was used as is.

¹H NMR (500 MHz, CDCl₃) ppm (δ): 7.40 (s, 1H), 7.36 (m, 2H), 7.24 (d, 1H), 4.26 (t, 4H), 3.84 (t, 4H), 2.40 (m, 4H).

### Preparative Example 28

### Preparation of 5-(3-CHLOROPROPOXY)-1-BENZOTHIOPHENE-6-OL and 6-(3-CHLOROPROPOXY-1-BENZOTHIOPHENE-5-OL

To a stirred solution of 1-benzothiophene-5,6-diol (0.51 g, 3.1 mmol) in anhydrous DMF (6 mL) was added cesium carbonate (1.2 g, 3.7 mmol) at 0° C. under nitrogen followed by dropwise addition of 1-bromo-3-chloropropane (0.33 mL, 3.4 mmol). After the addition, the reaction was allowed to warm to ambient temperature and stirred for 22 h. The reaction mixture was filtered to remove excess cesium carbonate. The filtrate was partitioned between EtOAc and 2 N HCl/ice. The organic layer was collected, washed with brine, dried over sodium sulfate, and concentrated in vacuo. The residue was purified by chromatography using the Isco CombiFlash Companion (12 g, column, 0% to 100% EtOAc/hexane over 28 column volume; flow rate=30 mL/min; desired product elutes at 48% EtOAc/hexane) to give the product as a 2:1 mixture of regioisomers. Further purification by Chiracel Semi-Prep OD column (flow rate=9 mL/min; 15% EtOH/heptane for 28 min.; 220 nM; 13 injections) separated the regioisomers as white solids.

Major isomer (0.1966 g): ¹H NMR (500 MHz, CDCl₃) δ (ppm): 7.35 (2s, 2H), 7.30 (d, 1H), 7.20 (d, 1H), 4.31 (t, 4H), 3.79 (t, 4H), 2.36 (m, 4H).

Minor isomer (0.0732 g): ¹H NMR (500 MHz, CDCl₃) δ (ppm): 7.41 (s, 2H), 7.28 (s, 1H), 7.28 (d, 1H), 7.21 (d, 1H), 4.32 (t, 4H), 3.79 (t, 4H), 2.36 (m, 4H).

### Preparative Example 29

### 6-(4-CHLOROBUTOXY)-5-(3-CHLOROPROPOXY)-1-BENZOTHIOPHENE and 5-(4-CHLOROBUTOXY-6-(3-CHLOROPROPOXY)-1-BENZOTHIOPHENE

Utilizing the procedure described above, the two isomers, from the previous example, were separately treated with 1-bromo-4-chlorobutane to give the desire products after purification by chromatography using the Isco CombiFlash Companion (4 g, column, 0% to 70% EtOAc/hexane over 40 column volume; flow rate=18 mL/min; desired product elutes at 20% EtOAc/hexane).

Major isomer (0.23 g): ¹H NMR (500 MHz, CDCl₃) δ (ppm): 7.38 (s, 1H), 7.31 (d, 1H), 7.29 (s, 1H), 7.22 (d, 1H), 4.23 (t, 2H), 4.11 (t, 2H), 3.81 (t, 2H), 3.71 (t, 2H), 2.34 (m, 2H), 2.04 (m, 4H).

Minor isomer (0.0838 g): ¹H NMR (500 MHz, CDCl₃) δ (ppm): 7.34 (s, 1H), 7.32 (s, 1H), 7.31 (d, 1H), 7.22 (d, 1H), 4.23 (t, 2H), 4.11 (t, 2H), 3.82 (t, 2H), 3.70 (t, 2H), 2.34 (m, 2H), 2.04 (m, 4H).

### Preparative Example 30

### Preparation of 5-(2-CHLOROETHOXY)-3-METHYL-1-BENZOTHIOPHENE

To an acetone solution (180 mL) of 3-methyl-5-hydroxy-1-benzothiophene (3.56 g, 23.73 mmol) was added cesium carbonate (30.1 g, 92.5 mmol), 1-bromo-2-chloroethane (5.9 mL, 71.2 mmol) and 0.5 mL of deionized water. The suspension was stirred vigorously at 60° C. for 4.0 hrs. The mixture was cooled to room temperature and the white precipitate was removed by filtration. The filtrate was diluted with EtOAc and washed with water and brine. The organic layer was dried over anhydrous Na₂SO₄, filtered and evaporated in vacuo. The crude residue was purified by silica gel chromatography using a gradient elution with 5% EtOAc-hexanes to 100% EtOAc to give the product.

¹H NMR (500 MHz, CDCl₃) [symbol] ppm): 7.95 (d, J=8.7 Hz, 1H), 7.49 (d, J=5.2 Hz, 1H), 7.33 (d, J=2.5 Hz, 1H), 7.29 (d, J=5.2 Hz, 1H), 7.07 (dd, J=2.3 Hz, 8.8 Hz, 1H), 4.34 (t, J=6.0 Hz, 2H), 3.89 (t, J=6.0 Hz, 2H).

Utilizing the above procedures and the appropriate alkylating agent the following compounds were prepared:

5,6-bis(4-chlorobutoxy)-1-benzothiophene was prepared and purified using 5% EtOAc/hexane as the chromatography eluant on a short pad silica gel.

¹H NMR (500 MHz, CDCl₃) δ (ppm): 7.3 (s, 1H), 7.3 (d, 1H), 7.3 (s, 1H), 7.2 (d, 1H), 4.2 (t, 4H), 3.7 (t, 4H), 2.1 (m, 8H).

5,6-bis(3-chloropropoxy)-3-methyl-1-benzothiophene

¹H NMR (500 MHz, CDCl₃) δ (ppm): 7.4 (s, 1H), 7.2 (s, 1H), 7.0 (s, 1H), 4.35 (m, 4H), 3.85 (m, 4H), 2.45 (s, 3H), 2.35 (m, 4H); MS m/z 334 (M+1).

5,6-bis(3-chloropropoxy)-3-ethyl-1-benzothiophene

Purified by silica gel chromatography (EtOAc/Hexane=1/9).

¹H NMR (500 MHz, CDCl₃) δ (ppm): 7.40 (s, 1H), 7.22 (s, 1H), 7.00 (d, 1H), 4.22 (m, 4H), 3.82 (m, 4H), 2.82 (q, 2H), 2.35 (m, 4H), 1.40 (t, 3H).

5,6-bis(3-chloropropoxy)-4,7-difluoro-1-benzothiophene

¹H NMR (500 MHz, CDCl₃) δ (ppm): 7.42 (m, 2H), 4.35 (m, 4H), 3.85 (m, 4H), 2.35 (m, 4H); MS m/z 357 (M+1).

5,6-bis(3-chloropropoxy)-4,7-difluoro-3-methyl-1-benzothiophene

¹H NMR (500 MHz, CDCl₃) δ (ppm): 6.99 (s, 1H), 4.4 (two t, 4H), 3.85 (m, 4H), 2.6 (s, 3H), 2.25 (m, 4H).

5,6-bis(3-chloropropoxy)-4,7-difluoro-3-ethyl-1-benzothiophene

Purified by silica gel chromatography (EtOAc/Hexane=1/9).

¹H NMR (500 MHz, CDCl₃) δ (ppm): 7.00 (s, 1H), 4.35 (t, 2H), 4.30 (t, 2H), 3.86 (m, 4H), 3.00 (q, 2H), 2.28 (m, 4H), 1.35 (t, 3H).

### Preparative Example 31

### 5-(MORPHIN-4-YLMETHYL)-1-BENZOTHIOPHENE-2-SULFONAMIDE

To a solution of 4-(1-benzothien-5-ylmethyl)morpholine (1.7 g, 7.29 mmol, 1 eq) in anhydrous tetrahydrofuran (36 mL) at −78° C. was slowly added n-butyllithium (3.50 mL, 2.5 M solution in hexane, 8.75 mmol, 1.20 eq). The reaction mixture was allowed to warm to −40° C., placed under SO₂ and slowly warmed up to room temperature. The SO₂ was removed and the reaction mixture was concentrated. The residue was suspended in dichloromethane (45 mL) and treated with N-chlorosuccinimide (1.12 g, 8.38 mmol, 1.15 eq). After stirring for 1 hour at room temperature, the reaction mixture was filtered through celite and concentrated. The residue was dissolved in acetone (35 mL), treated with ammonium hydroxide (7 mL) for 1 hour and concentrated. The residue was adsorbed on silica gel and purified by flash chromatography on silica gel (methanol/dichloromethane, 6:94). The resulting solid was triturated with dichloromethane, filtered off, rinsed with dichloromethane and dried under high vacuum to afford 5-(morpholin-4-ylmethyl)-1-benzothiophene-2-sulfonamide as a white solid.

¹H NMR (400 MHz, DMSO-d₆) δ (ppm): 7.98 (d, J=8.4 Hz, 1H), 7.90 (s, 1H), 7.87 (s, 1H), 7.45 (d, J=8.4 Hz, 1H), 3.60-3.55 (m, 6H), 2.40-30 (m, 4H); MS m/z 313 (M+1).

Following the foregoing general procedure, the following compounds were prepared:

5,6-bis(4-chlorobutoxy)-1-benzothiophene-2-sulfonamide was purified using 30% EtOAc/hexane as the chromatography eluant.

¹H NMR (500 MHz, CD₃OD) δ (ppm): 7.75 (s, 1H), 7.46 (s, 1H), 7.4 (s, 1H), 4.18 (m, 4H), 3.70 (t, 4H), 2.00 (m, 8H).

5,6-bis(3-chloropropoxy)-1-benzothiophene-2-sulfonamide was purified using 30% EtOAc/hexane as the chromatography eluant.

¹H NMR (500 MHz, CD₃OD) δ (ppm): 7.75 (s, 1H), 7.46 (s, 1H), 7.42 (s, 1H), 4.20 (m, 4H), 3.82 (t, 4H), 2.30 (m, 4H).

5-(4-chlorobutoxy)-6-(3-chloropropoxy)-1-benzothiophene-2-sulfonamide and 6-(4-chlorobutoxy)-5-(3-chloropropoxy)-1-benzothiophene-2-sulfonamide were prepared. Purification was accomplished by chromatography using the Isco CombiFlash Companion (4 g, column, 10% to 100% EtOAc/hexane over 30 column volume; flow rate=18 mL/min; desired product elutes at 68% EtOAc/hexane).

Major isomer (0.2066 g): ¹H NMR (500 MHz, CDCl₃) δ (ppm): 7.81 (s, 1H), 7.31 (s, 1H), 7.28 (s, 1H), 5.05 (bs, 2H), 4.27 (t, 2H), 4.13 (t, 2H), 3.85 (t, 2H), 3.73 (t, 2H), 2.39 (m, 2H), 2.08 (m, 4H).

Minor isomer (0.0657 g): NMR (500 MHz, CDCl₃) δ (ppm): 7.79 (s, 1H), 7.29 (s, 1H), 7.25 (s, 1H), 5.11 (bs, 2H), 4.22 (t, 2H), 4.13 (t, 2H), 3.83 (t, 2H), 3.71 (t, 2H), 2.34 (m, 2H), 2.06 (m, 4H).

5,6-bis(2-chloroethoxy)-1-benzothiophene-2-sulfonamide

¹H NMR (400 MHz, CDCl₃) δ (ppm): 7.29 (s, 1H), 7.26 (s, 1H), 7.24 (s, 1H), 4.25 (m, 4H), 3.80 (m, 4H);

MS m/z 291 (M+1).

5,6-bis(2-chloroethoxy)-4,7-difluoro-1-benzothiophene-2-sulfonamide

¹H NMR (400 MHz, CDCl₃) δ (ppm): 7.85 (m, 1H), 4.4 (m, 4H), 3.80 (m, 4H); MS m/z 407 (M+1).

5,6-bis(2-chloroethoxy)-3-methyl-4,7-difluoro-1-benzothiophene-2-sulfonamide

Purification was accomplished by chromatography using the Biotage Horizon (25 M column, 10% to 100% EtOAc/hexane over 10 column volumes; flow rate=25 mL/min) to give the desired product as a pale yellow solid.

¹H NMR (500 MHz, CD₃OD) δ (ppm): 4.49 (t, 2H), 4.42 (t, 2H), 3.90 (t, 4H), 2.78 (s, 3H).

5,6-bis(2-chloroethoxy)-3-ethyl-4,7-difluoro-1-benzothiophene-2-sulfonamide

Purified by silica gel chromatography (EtOAc/Hexane=2/3).

¹H NMR (500 MHz, CD₃OD) δ (ppm): 4.50 (t, 2H), 4.44 (t, 2H), 3.90 (m, 4H), 3.24 (q, 2H), 1.36 (t, 3H).

5,6-bis(2-chloroethoxy)-3-methyl-1-benzothiophene-2-sulfonamide

Purification was accomplished by chromatography using the Isco CombiFlash Companion (12 g, column, 5% to 100% EtOAc/hexane over 12 min.; flow rate=30 mL/min; desired product elutes at 65% EtOAc/hexane).

¹H NMR (500 MHz, CD₃OD) δ (ppm): 7.49 (s, 1H), 7.40 (s, 1H), 4.37 (m, 4H), 3.91 (m, 4H), 2.61 (s, 3H).

5,6-bis(3-chloroproxy)-3-methyl-1-benzothiophene-2-sulfonamide was prepared.

¹H NMR (500 MHz, CD₃OD) δ (ppm): 7.45 (s, 1H), 7.34 (s, 1H), 4.25 (m, 4H), 3.85 (m, 4H), 2.65 (s, 3H); 2.3 (m, 4H); MS m/z 412 (M+1).

5,6-bis(3-chloroproxy)-3-ethyl-1-benzothiophene-2-sulfonamide

Purified by silica gel chromatography (EtOAc/Hexane=2/3).

¹H NMR (500 MHz, CD₃OD) δ (ppm): 7.45 (s, 1H), 7.35 (s, 1H), 4.22 (m, 4H), 3.82 (m, 4H), 3.20 (q, 2H), 2.30 (m, 4H), 1.30 (t, 3H).

5,6-bis(3-chloroproxy)-4,7-difluoro-3-methyl-1-benzothiophene-2-sulfonamide was prepared in 69% yield.

¹H NMR (500 MHz, CDCl₃) δ (ppm): 4.4 (two t, 4H), 3.85 (m, 4H); 2.8 (s, 3H), 2.3 (m, 4H); MS m/z 448 (M+1).

5,6-bis(3-chloroproxy)-4,7-difluoro-3-ethyl-1-benzothiophene-2-sulfonamide

Purified by silica gel chromatography (EtOAc/Hexane=2/3).

¹H NMR (500 MHz, CD₃OD) δ (ppm): 4.40 (t, 2H), 4.30 (t, 2H), 3.82 (m, 4H), 3.22 (q, 2H), 2.26 (m, 4H), 1.36 (t, 3H).

5,6-bis(3-chloroproxy)-4,7-difluorol-1-benzothiophene-2-sulfonamide was prepared in 82% yield.

¹H NMR (500 MHz, CD₃OD) δ (ppm): 7.9 (d, 1H), 4.24 (m, 4H), 3.85 (m, 4H); 22 (m, 4H); MS m/z 434 (M+1).

### Preparative Example 32

### Preparation of TERT-BUTYL({5-[3,4-BIS(3-CHLOROPROPOXY)PHENYL]THIOPHENE-2-YL}SULFONYL)CARBAMATE

To a stirred solution of the sulfonamide (2.7 g, 6.9 mmol) in anhydrous dichloromethane (35 mL) at room temperature was added 4-(dimethylamino)pyridine (85 mg, 0.10 equiv) followed by triethylamine (3.7 mL, 4 equiv) and di-tert-butyl dicarbonate (1.75 g, 1.2 equiv). After 2 h, the reaction mixture was concentrated and the residue was purified by silica gel chromatography (5% of MeOH in CH₂Cl₂) to provide the product (1.8 g, 32% overall for 5 steps).

¹H NMR (500 MHz, CD₃OD) δ (ppm): 7.70 (d, 1H), 7.32 (d, 1H), 7.04 (m, 2H), 7.00 (d, 1H), 4.20 (m, 4H), 3.80 (m, 4H), 2.22 (m, 4H), 1.42 (s, 9H).

Utilizing the foregoing procedures, the following compounds were prepared:

tert-butyl({5-[3,4-bis(3-chloropropoxy)-2-methylphenyl]thiophen-2-yl}sulfonyl)carbamate was purified by silica gel chromatography (5% MeOH in CH₂Cl₂).

¹H NMR (500 MHz, CD₃OD) δ (ppm): 7.78 (d, 1H), 7.20 (d, 1H), 7.10 (d, 1H), 7.00 (d, 1H), 4.20 (t, 2H), 4.10 (t, 2H), 3.82 (m, 4H), 2.34 (s, 3H), 2.24 (m, 4H), 1.44 (s, 9H).

tert-butyl({5-[4,5-bis(3-chloropropoxy)-2-methylphenyl]thiophen-2-yl}sulfonyl)carbamate was purified by silica gel chromatography (5% MeOH in CH₂Cl₂).

¹H NMR (500 MHz, CD₃OD) δ (ppm): 7.78 (d, 1H), 7.10 (d, 1H), 7.00 (s, 1H), 6.98 (s, 1H), 4.20 (t, 2H), 4.10 (t, 2H), 3.80 (m, 4H), 2.36 (s, 3H), 2.22 (m, 4H), 1.44 (s, 9H).

tert-butyl {[5,6-bis(4-chlorobutoxy)-1-benzothien-2-yl]sulfonyl}carbamate was purified by silica gel chromatography using 5% MeOH/CH₂Cl₂ as the eluant.

¹H NMR (500 MHz, CD₃OD) δ (ppm): 8.00 (s, 1H), 7.40 (s, 1H), 7.30 (s, 1H), 7.25 (s, 1H), 4.18 (m, 4H), 3.72 (t, 4H), 2.15 (m, 8H), 1.40 (s, 9H).

tert-butyl {[5,6-bis(3-chloropropoxy)-1-benzothien-2-yl]sulfonyl}carbamate was purified by silica gel chromatography using 5% MeOH/CH₂Cl₂ as the eluant.

¹H NMR (500 MHz, CD₃OD) δ (ppm): 7.95 (s, 1H), 7.52 (s, 1H), 7.50 (s, 1H), 4.26 (m, 4H), 3.84 (t, 4H), 2.30 (m, 4H), 1.40 (s, 9H).

tert-butyl {[5,6-bis(3-chloroethoxy)-1-benzothien-2-yl]sulfonyl}carbamate was prepared in >90% yield, as a colorless oil.

¹H NMR (400 MHz, CDCl₃) δ (ppm): 8.0 (s, 1H), 7.8 (s, 1H), 7.4 (s, 1H), 7.35 (s, 1H), 4.4 (m, 4H), 3.90 (m, 4H).

tert-butyl {[5,6-bis(2-chloroethoxy)-4,7-difluoro-1-benzothien-2-yl]sulfonyl}carbamate

¹H NMR (400 MHz, CDCl₃) δ (ppm): 8.1 (d, 1H), 7.8 (s, 1H), 4.5 (t, 2H), 4.45 (t, 2H), 3.85 (m, 4H), 1.5 (s, 9H).

tert-butyl {[5,6-bis(3-chloropropoxy)-3-methyl-1-benzothien-2-yl]sulfonyl}carbamate was prepared in 81% yield.

¹H NMR (500 MHz, CD₃OD) [symbol] (ppm): 7.5 (brs, 1H), 7.28 (s, 1H), 7.24 (s, 1H), 4.25 (t, 4H), 3.8 (q, 4H); 2.7 (s, 3H), 2.4 (m, 4H), 1.4 (s, 9H).

tert-butyl {[5,6-bis(3-chloropropoxy)-3-ethyl-1-benzothien-2-yl]sulfonyl}carbamate

Purified by silica gel chromatography (MeOH/CH₂Cl₂=0.5/9.5).

¹H NMR (500 MHz, CD₃OD) δ (ppm): 7.50 (s, 1H), 7.40 (s, 1H), 4.25 (m, 4H), 3.82 (m, 4H), 3.20 (q, 2H), 2.30 (m, 4H), 1.40 (s, 9H), 1.30 (t, 3H).

tert-butyl {[5,6-bis(3-chloropropoxy)-4,7-difluoro-3-methyl-1-benzothien-2-yl]sulfonyl}-carbamate was prepared in 100% yield.

¹H NMR (500 MHz, CDCl₃) δ (ppm): 4.4 (two t, 4H), 3.82 (m, 4H), 2.85 (s, 3H), 2.3 (m, 4H), 1.5 (s, 9H).

tert-butyl {[5,6-bis(2-chloroethoxy)-4,7-difluoro-3-ethyl-1-benzothien-2-yl]sulfonyl}carbamate

¹H NMR (500 MHz, CD₃OD) δ (ppm): 4.50 (t, 2H), 4.40 (t, 2H), 3.86 (m, 4H), 3.28 (q, 2H), 1.40 (s, 9H), 1.30 (t, 3H).

tert-butyl {[5,6-bis(3-chloropropoxy)-4,7-difluoro-3-ethyl-1-benzothien-2-yl]sulfonyl}carbamate

¹H NMR (500 MHz, CD₃OD) δ (ppm): 4.42 (t, 2H), 4.32 (t, 2H), 3.86 (m, 4H), 3.28 (q, 2H), 2.26 (m, 4H), 1.40 (s, 9H), 1.30 (t, 3H).

tert-butyl {[5,6-bis(3-chloropropoxy)-4,7-difluoro-1-benzothien-2-yl]sulfonyl}carbamate

¹H NMR (500 MHz, CDCl₃) δ (ppm): 8.1 (d, 1H), 8.2 (d, 1H), 4.4 (two t, 4H), 3.82 (t, 4H), 2.25 (m, 4H), 1.5 (s, 9H).

### Preparative Example 33

### Preparation of DIETHYL {[{[5,6-BIS(2-CHLOROETHOXY)-1-BENZOTHIEN-2-YL]-SULFONYL}(TERT-BUTOXYCARBONYL)AMINO]METHYL}PHOSPHONATE

The N-Boc sulfonamide (2.54 g, 5.4 mmol), triphenylphosphine resin (7.2 g, 3 equiv), and diethyl-hydroxymethylphosphonate (2.5 mL, 3 equiv) were combined and stirred in anhydrous THF. The stirred mixture was cooled to 0° C. and diisopropyl azodicarboxylate (4.3 mL, 3 equiv) was added dropwise over a 0.2 h period. The resulting pale yellow solution was stirred at room temperature overnight. The volatile components were then removed in vacuo and the residue purified by silica gel flash chromatography (EA/Hex=1:2, followed by 3% MeOH/CH₂Cl₂) to give the product which was contaminated with a small amount of diethyl-hydroxymethylphosphonate).

¹H NMR (400 MHz, CDCl₃) δ (ppm): 8.0 (s, 1H), 7.35 (s, 1H), 7.3 (s, 1H), 4.3 (m, 4H), 4.25 (d, 2H), 4.1 (m, 4H), 3.85 (m, 4H), 1.4 (s, 9H), 1.3 (t, 6H).

Utilizing the foregoing procedures, the following compounds were prepared:

diethyl-{[(tert-butoxycarbonyl)({5-[3,4-bis(3-chloropropoxy)phenyl]thiophen-2-yl}sulfonyl)-amino]methyl}phosphonate was prepared and purified by silica gel chromatography (5% MeOH in CH₂Cl₂).

¹H NMR (500 MHz, CD₃OD) δ (ppm): 7.80 (d, 1H), 7.60 (d, 1H), 7.30 (m, 2H), 7.08 (d, 1H), 4.30 (d, 2H), 4.20 (m, 8H), 3.80 (m, 4H), 2.22 (m, 4H), 1.42 (s, 9H), 1.36 (m, 6H).

diethyl-{[(tert-butoxycarbonyl)({5-[3,4-bis(3-chloropropoxy)-2-methylphenyl]thiophen-2-yl}-sulfonyl)-amino]methyl}phosphonate was prepared and purified by silica gel chromatography (5% MeOH in CH₂Cl₂).

¹H NMR (500 MHz, CD₃OD) δ (ppm): 7.80 (d, 1H), 7.12 (d, 1H), 7.08 (d, 1H), 6.98 (d, 1H), 4.30 (d, 2H), 4.20 (m, 6H), 4.10 (t, 2H), 3.80 (m, 4H), 2.30 (s, 3H), 2.22 (m, 4H), 1.42 (s, 9H), 1.36 (m, 6H).

diethyl-{[(tert-butoxycarbonyl)({5-[4,5-bis(3-chloropropoxy)-2-methylphenyl]thiophen-2-yl}-sulfonyl)-amino]methyl}phosphonate was prepared and purified by silica gel chromatography (5% MeOH in CH₂Cl₂).

¹H NMR (500 MHz, CD₃OD) δ (ppm): 7.84 (d, 1H), 7.14 (d, 1H), 7.00 (s, 1H), 6.98 (s, 1H), 4.30 (d, 2H), 4.20 (m, 8H), 3.80 (m, 4H), 2.40 (s, 3H), 2.22 (m, 4H), 1.42 (s, 9H), 1.34 (m, 6H).

diethyl-{[{[5,6-bis(4-chlorobutoxy)-1-benzothien-2-yl]sulfonyl}(tert-butoxycarbonyl)amino]-methyl}phosphonate was prepared and purified using 5% MeOH/CH₂Cl₂ as the chromatography eluant.

¹H NMR (500 MHz, CDCl₃) δ (ppm): 8.08 (s, 1H), 7.50 (s, 1H), 7.46 (s, 1H), 4.35 (d, 2H), 4.20 (m, 8H), 330 (m, 4H), 2.05 (m, 8H), 1.42 (s, 9H), 1.38 (t, 6H); MS m/z 576 (M−100).

diethyl-{[{[5,6-bis(3-chloropropoxy)-1-benzothien-2-yl]sulfonyl}(tert-butoxycarbonyl)amino]-methyl}phosphonate was prepared in 89% yield and purified using 5% MeOH/CH₂Cl₂ as the chromatography eluant.

¹H NMR (500 MHz, CDCl₃) δ (ppm): 8.08 (s, 1H), 7.32 (s, 1H), 7.30 (s, 1H), 4.30 (d, 2H), 4.20 (m, 8H), 3.82 (t, 4H), 2.38 (m, 4H), 1.42 (s, 9H), 1.38 (t, 6H); MS m/z 548 (M−100).

diethyl {[{[5,6-bis(2-chloroethoxy)-4,7-difluoro-1-benzothien-2-yl]sulfonyl}(tert-butoxycarbonyl)amino]methyl}phosphonate

¹H NMR (400 MHz, CDCl₃) δ (ppm): 8.22 (d, 1H), 4.55 (t 2H), 4.45 (t, 2H), 4.3 (d, 2H), 4.2 (m, 4H), 1.5 (s, 9H), 1.4 (t, 6H).

diethyl {[{[5,6-bis(2-chloroethoxy)-4,7-difluoro-3-ethyl-1-benzothien-2-yl]sulfonyl}(tert-butoxycarbonyl)amino]methyl}phosphonate

Purified by silica gel chromatography (EtOAc/Hexane=3/7).

¹H NMR (500 MHz, CD₃OD) δ (ppm): 4.58 (t, 2H), 4.44 (t, 2H), 4.40 (d, 2H), 4.25 (m, 4H), 3.90 (m, 4H), 3.14 (q, 2H), 1.40 (m, 15H), 1.30 (t, 3H); MS m/z 585 (M−100).

diethyl {[{[5,6-bis(3-chloropropoxy)-3-methyl-1-benzothien-2-yl]sulfonyl}(tert-butoxycarbonyl)amino]methyl}phosphonate

¹H NMR (500 MHz, CDCl₃) δ (ppm): 7.27 (s, 1H), 7.22 (s, 1H), 4.4 (d, 2H), 4.2 (m, 4H), 3.8 (m, 4H), 2.6 (s, 3H), 2.4 (m, 4H), 1.55 (m, 6H), 1.5 (s, 9H).

diethyl {[{[5,6-bis(3-chloropropoxy)-3-ethyl-1-benzothien-2-yl]sulfonyl}(tert-butoxycarbonyl)amino]methyl}phosphonate

Purified by silica gel chromatography (EtOAc/Hexane=3/7).

¹H NMR (500 MHz, CD₃OD) δ (ppm): 7.50 (s, 1H), 7.40 (s, 1H), 4.20 (m, 8H), 3.85 (d, 2H), 3.80 (m, 4H), 3.10 (q, 2H), 2.30 (m, 4H), 1.40 (s, 9H), 1.30 (m, 9H); MS m/z 576 (M−100).

diethyl {[{[4,7-difluoro-5,6-bis(3-chloropropoxy)-3-ethyl-1-benzothien-2-yl]sulfonyl}(tert-butoxycarbonyl)amino]methyl}phosphonate

Purified by silica gel chromatography (EtOAc/Hexane=3/7).

¹H NMR (500 MHz, CD₃OD) δ (ppm): 4.40 (t, 2H), 4.30 (t, 2H), 4.30 (m, 8H), 3.85 (d, 2H), 3.82 (m, 4H), 3.14 (q, 2H), 2.42 (m, 4H), 1.40 (s, 9H), 1.35 (m, 9H); MS m/z 712 (M−100).

### Preparative Example 34

### Method A. Preparation of [({[5,6-BIS(2-CHLOROETHOXY)-1-BENZOTHIEN-2-YL]SULFONYL}AMINO)METHYL]PHOSPHONIC ACID

To a stirred solution of the sulfonamide (0.52 g, 1.39 mmol) in 6 mL of Ac₂O/AcOH (2:1) was added paraformaldehyde (51 mg, 1.2 equiv). The reaction mixture was heated at 75° C. until all of the solid was dissolved, and then P(OTMS)₃ (0.49 mL, 1.05 equiv) was added. The homogeneous solution was heated at 110° C. for 3 h, then cooled to room temperature, and concentrated. The residue was dissolved in dichloromethane at room temperature and TMSBr (1.8 mL, 8 equiv) was added. The mixture was left overnight and the volatiles were removed by evaporation. The oily residue so obtained was dissolved in MeOH and stirred with 5 equivalents of K₂CO₃ at room temperature overnight. The mixture was filtered, neutralized with dilute HCl, and purified by HPLC to give the product.

¹H NMR (400 MHz, CD₃OD) δ (ppm): 7.8 (s, 1H), 7.55 (s, 1H), 7.52 (s, 1H), 4.4 (m, 4H), 3.95 (m, 4H), 3.25 (brd, 2H); MS m/z 464 (M+1).

Utilizing the foregoing procedure, the following compounds were prepared:

[({[6-(4-chlorobutoxy)-5-(3-chloropropoxy)-1-benzothien-2-yl]sulfonyl}amino)methyl]-phosphonic acid

The minor isomer was elaborated to its corresponding phosphonic acid.

¹H NMR (500 MHz, CD₃OD) δ (ppm): 7.79 (s, 1H), 7.47 (s, 2H), 4.21 (t, 2H), 4.13 (t, 2H), 3.82 (t, 2H), 3.71 (t, 2H), 3.02 (d, 2H), 2.27 (m, 2H), 2.02 (m, 4H); MS m/z 506 (M).

[({[5,6-bis(2-chloroethoxy)-3-methyl-1-benzothien-2-yl]sulfonyl}amino)methyl]phosphonic acid

¹H NMR (500 MHz, CD₃OD) δ (ppm): 7.52 (s, 1H), 7.43 (s, 1H), 4.39 (m, 4H), 3.92 (m, 4H), 3.03 (d, 2H), 2.65 (s, 3H).

[({[5,6-bis(2-chloroethoxy)-3-methyl-4,7-difluoro-1-benzothien-2-yl]sulfonyl}amino)methyl]-phosphonic acid

¹H NMR (500 MHz, CD₃OD) δ (ppm): 4.50 (t, 2H), 4.41 (t, 2H), 3.84 (m, 4H), 3.22 (d, 2H), 2.80 (s, 3H);

MS m/z 528 (M+1).

### Method B. Preparation of DIETHYL {[{[5,6-BIS(2-CHLOROETHOXY)-1-BENZOTHIEN-2-YL]-SULFONYL}AMINO]METHYL}PHOSPHONATE

To a stirred solution of the N-Boc-phosphonate in CH₂Cl₂ at 0° C. was added drop wise TFA and the mixture was warmed up to room temperature. After 1 h, a saturated, aqueous solution of NaHCO₃ was added to quench the excess TFA. The organic layer was separated and the aqueous layer was extracted with ethyl acetate. The organic layers were combined, dried over magnesium sulfate, filtered, and evaporated in vacuo. Purification by silica gel chromatography yielded the product.

¹H NMR (400 MHz, CDCl₃) δ (ppm): 7.8 (s, 1H), 7.4 (s, 1H), 7.35 (s, 1H), 5.6 (m, 1H), 4.4 (m, 4H), 4.2 (m, 4H), 3.95 (m, 4H), 3.4 (dd, 2H), 1.4 (t, 6H).

Utilizing the foregoing procedure, the following compounds were prepared:

diethyl[({[5,6-bis(4-chlorobutoxy)-1-benzothien-2-yl]sulfonyl}amino)methyl]phosphonate was prepared and used as is for next step.

¹H NMR (500 MHz, CD₃OD) δ (ppm): 7.80 (s, 1H), 7.24 (s, 1H), 7.22 (s, 1H), 4.16 (m, 8H), 3.70 (m, 4H), 3.40 (d, 2H), 2.00 (m, 8H), 1.32 (t, 6H); MS m/z 576 (M).

diethyl[({[5,6-bis(3-chloropropoxy)-1-benzothien-2-yl]sulfonyl}amino)methyl]phosphonate was prepared and purified using 5% MeOH/CH₂Cl₂ as the chromatography eluant.

¹H NMR (500 MHz, CD₃OD) δ (ppm): 7.82 (s, 1H), 7.32 (s, 1H), 7.30 (s, 1H), 4.24 (q, 4H), 4.18 (m, 4H), 3.82 (t, 4H), 3.20 (d, 2H), 2.30 (m, 4H), 1.32 (t, 6H); MS m/z 548 (M).

diethyl[({[5,6-bis(3-chloropropoxy)-3-ethyl-1-benzothien-2-yl]sulfonyl}amino)methyl]-phosphonate

¹H NMR (500 MHz, CD₃OD) δ (ppm): 7.45 (s, 1H), 7.35 (s, 1H), 4.20 (m, 4H), 4.10 (m, 4H), 3.80 (m, 4H), 3.40 (d, 2H), 3.18 (q, 2H), 2.25 (m, 4H), 1.25 (m, 9H); MS m/z 576 (M).

diethyl-[({[5,6-bis(3-chloropropoxy)-4,7-difluoro-1-benzothien-2-yl]sulfonyl}amino)methyl]-phosphonate

¹H NMR (500 MHz, CDCl₃) δ (ppm): 7.9 (d, 1H), 7.63 (br m, 1H), 4.35 (two t, 4H), 4.2 (m, 4H), 3.8 (m, 4H), 3.4 (dd, 2H), 2.25 (m, 4H), 1.35 (t, 6H); MS m/z 584 (M+1).

diethyl-M[({[5,6-bis(3-chloropropoxy)-4,7-difluoro-3-methyl-1-benzothien-2-yl]-sulfonyl}amino)methyl]phosphonate

¹H NMR (500 MHz, CDCl₃) δ (ppm): 4.35 (m, 4H), 4.2 (m, 4H), 3.8 (m, 4H), 3.4 (brd, 2H), 2.8 (s, 3H), 2.25 (m, 4H), 1.4 (t, 6H); MS m/z 584 (M+1).

diethyl-[({[5,6-bis(2-chloroethoxy)-4,7-difluoro-3-ethyl-1-benzothien-2-yl]-sulfonyl}amino)methyl]phosphonate

¹H NMR (500 MHz, CD₃OD) δ (ppm): 4.54 (t, 2H), 4.42 (t, 2H), 4.18 (m, 4H), 3.90 (m, 4H), 3.50 (d, 2H), 3.25 (q, 2H), 1.30 (m, 9H); MS m/z 584 (M).

diethyl-[({[5,6-bis(3-chloropropoxy)-4,7-difluoro-3-ethyl-1-benzothien-2-yl]-sulfonyl}amino)methyl]phosphonate

¹H NMR (500 MHz, CD₃OD) δ (ppm): 4.40 (t, 2H), 4.30 (t, 2H), 4.20 (m, 8H), 3.88 (m, 4H), 3.50 (d, 2H), 3.25 (q, 2H), 2.28 (m, 4H), 1.30 (m, 9H); MS m/z 612 (M).

diethyl-{[({5-[3,4-bis(3-chloropropoxy)phenyl]thiophen-2-yl}sulfonyl)amino]methyl}-phosphonate was prepared and used as is in the next step.

¹H NMR (500 MHz, CD₃OD) δ (ppm): 7.60 (d, 1H), 7.35 (d, 1H), 7.28 (m, 2H), 7.08 (d, 1H), 4.20 (m, 8H), 3.80 (m, 4H), 3.40 (d, 2H), 2.22 (m, 4H), 1.36 (m, 6H).

diethyl-{[({5-[3,4-bis(3-chloropropoxy-2-methylphenyl]thiophen-2-yl}sulfonyl)amino]methyl}-phosphonate was prepared and used as is in the next step.

¹H NMR (500 MHz, CD₃OD) δ (ppm): 7.60 (d, 1H), 7.18 (d, 1H), 7.10 (d, 1H), 7.00 (d, 1H), 4.20 (m, 8H), 3.82 (m, 4H), 3.40 (d, 2H), 2.36 (s, 3H), 2.22 (m, 4H), 1.36 (m, 6H).

diethyl-{[({5-[4,5-bis(3-chloropropoxy-2-methylphenyl]thiophen-2-yl}sulfonyl)amino]methyl}-phosphonate was prepared and used as is in the next step.

¹H NMR (500 MHz, CD₃OD) δ (ppm): 7.62 (d, 1H), 7.18 (d, 1H), 7.04 (s, 1H), 6.98 (s, 1H), 4.20 (m, 8H), 3.82 (m, 4H), 3.40 (d, 2H), 2.40 (s, 3H), 2.22 (m, 4H), 1.26 (m, 6H).

### Method C. Preparation of DIETHYL {[{[4,7-DICHLORO-5,6-BIS(2-CHLOROETHOXY)-1-BENZOTHIEN-2-YL]SULFONYL}AMINO]METHYL}-PHOSPHONATE

To a stirred solution of diethyl-{[{[5,6-bis(2-chloroethoxy)-1-benzothien-2-yl]sulfonyl}-amino]methyl}phosphonate (3.3 g, 6.33 mmol) in CH₂Cl₂/HOAc (1:1) at room temperature was added NCS (5.07 g, 4 equiv) and the mixture was heated at 70° C. After 2 h, the reaction mixture was quenched with a saturated, aqueous solution of NaHCO₃ and the organic layer was separated, dried, and evaporated to give an oily residue. The residue was purified by silica gel chromatography to afford the product.

¹H NMR (400 MHz, CDCl₃) δ (ppm): 8.0 (s, 1H), 6.25 (brs, 1H), 4.4 (m, 4H), 4.2 (m, 4H), 3.95 (m, 4H); 3.42 (dd, 2H), 1.4 (m, 6H); MS m/z 590 (M+1).

### Method D. Preparation of [({[4,7-DICHLORO-5,6-BIS(2-CHLOROETHOXY)-1-BENZOTHIEN-2-YL]SULFONYL}AMINO)METHYL]PHOSPHONIC ACID

To a stirred solution of diethyl-{[{[4,7-dichloro-5,6-bis(2-chloroethoxy)-1-benzothien-2-yl]sulfonyl}amino]methyl}phosphonate (2 g, 3.38 mmol) in anhydrous dichloromethane at room temperature was added TMSBr (4.4 mL, 10 equiv). The mixture was left overnight and the dichloromethane was evaporated to give an oily residue. The residue was dissolved in MeOH and stirred at room temperature for 30 min. The mixture was evaporated and the residue was triturated with dichloromethane to form a crystalline product which was collected by filtration.

¹H NMR (400 MHz, CD₃OD) δ (ppm): 8.0 (s, 1H), 4.4 (m, 4H), 4.0 (m, 4H), 3.3 (d, 2H); MS m/z 534 (M+1).

Utilizing the foregoing procedure, the following compounds were prepared:

({[(5-formyl-1-benzothien-2-yl)sulfonyl]amino}methyl)phosphonic acid

¹H NMR (400 MHz, DMSO-d₆) δ (ppm): 10.10 (s, 1H); 8.58 (s, 1H); 8.46-8.42 (m, 1H); 8.30 (d, J=8.6, 1H); 8.19 (s, 1H); 7.98 (d, J=8.6, 1H); 3.02 (dd, J=13.1, 6.3, 2H).

[({[5,6-bis(3-chloropropoxy)-1-benzothien-2-yl]sulfonyl}amino)methyl]phosphonic acid was prepared and used as is in the next step.

MS m/z 492 (M).

[({[5,6-bis(3-chloropropoxy)-3-ethyl-1-benzothien-2-yl]sulfonyl}amino)methyl]phosphonic acid

¹H NMR (500 MHz, CD₃OD) δ (ppm): 7.50 (s, 1H), 7.40 (s, 1H), 4.24 (m, 4H), 3.82 (m, 4H), 3.25 (d, 2H), 3.20 (q, 2H), 2.30 (m, 4H), 1.30 (t, 3H); MS m/z 520 (M).

[({[4,7-difluoro-5,6-bis(2-chloroethoxy)-3-ethyl-1-benzothien-2-yl]sulfonyl}amino)methyl]phosphonic acid

¹H NMR (500 MHz, CD₃OD) δ (ppm): 4.50 (t, 2H), 4.42 (t, 2H), 3.92 (m, 4H), 3.32 (d, 2H), 3.26 (q, 2H), 1.30 (t, 3H); MS m/z 528 (M).

[({[4,7-difluoro-5,6-bis(3-chloropropoxy)-3-ethyl-1-benzothien-2-yl]sulfonyl}amino)methyl]phosphonic acid

¹H NMR (500 MHz, CD₃OD) δ (ppm): 4.40 (t, 2H), 4.30 (t, 2H), 3.82 (m, 4H), 3.30 (d, 2H), 3.22 (q, 2H), 2.25 (m, 4H), 1.30 (t, 3H); MS m/z 557 (M+1).

[({[5,6-bis(4-chlorobutoxy)-1-benzothien-2-yl]sulfonyl}amino)methyl]phosphonic acid was prepared and used as is in the next step.

¹H NMR (500 MHz, CD₃OD) δ (ppm): 7.80 (s, 1H), 7.24 (s, 1H), 7.22 (s, 1H), 4.10 (m, 4H), 3.70 (m, 4H), 3.20 (d, 2H), 2.00 (m, 8H); MS m/z 519 (M).

[({[5-(4-chlorobutoxy)-6-(3-chloropropoxy)-1-benzothien-2-yl]sulfonyl}amino)methyl]-phosphonic acid

The major isomer was elaborated to its corresponding phosphonic acid.

{[({5-[3,4-bis(3-chloropropoxy)phenyl]thiophen-2-yl}sulfonyl}amino)methyl]phosphonic acid was prepared and used as is in the next step.

¹H NMR (500 MHz, CD₃OD) δ (ppm): 7.60 (d, 1H), 7.35 (d, 1H), 7.24 (m, 2H), 7.04 (d, 1H), 4.20 (m, 4H), 3.80 (m, 4H), 3.20 (d, 2H), 2.22 (m, 4H); MS m/z 518 (M).

{[({5-[3,4-bis(3-chloropropoxy)-2-methylphenyl]thiophen-2-yl}sulfonyl)amino]methyl}-phosphonic acid was prepared and used as is in the next step.

¹H NMR (500 MHz, CD₃OD) δ (ppm): 7.60 (d, 1H), 7.20 (d, 1H), 7.10 (d, 1H), 7.00 (d, 1H), 4.20 (t, 2H), 4.10 (t, 2H), 3.80 (m, 4H), 3.24 (d, 2H), 2.36 (s, 3H), 2.22 (m, 4H).

{[({5-[4,5-bis(3-chloropropoxy)-2-methylphenyl]thiophen-2-yl}sulfonyl)amino]methyl}-phosphonic acid was prepared and used as is in the next step.

¹H NMR (500 MHz, CD₃OD) □ (ppm): 7.60 (d, 1H), 7.10 (d, 1H), 7.00 (s, 1H), 6.96 (s, 1H), 4.20 (t, 2H), 4.08 (t, 2H), 3.80 (m, 4H), 3.24 (d, 2H), 2.38 (s, 3H), 2.22 (m, 4H); MS m/z 561 (M).

(5,6-bis(2-chloroethoxy)benzo[d]thiazole-2-sulfonamido)methylphosphonic acid

¹H NMR (400 MHz, DMSO-d₆) δ (ppm): 8.78-8.75 (m, 1H); 7.86 (s, 1H); 7.79 (s, 1H); 4.40-4.34 (m, 4H); 4.00-3.96 (m, 4H); 3.22 (dd, J=12.7, 6.3, 2H).

5,6-bis(3-chloropropoxy)benzo[d]thiazole-2-sulfonamido]methylphosphonic acid

Off-white off solid.

¹H NMR (400 MHz, DMSO-d₆) [symbol] ppm): 7.74 (s, 1H), 7.73 (s, 1H), 4.29 (m, 4H), 3.87 (t, J=6.4 Hz, 4H), 3.33 (d, J=12.3 Hz, 2H), 2.33 (m, 4H); MS m/z 491 (M−1).

### Method E. Preparation of DIETHYL (5,6-BIS(2-CHLOROETHOXY)-BENZO[d]THIAZOLE-2-SULFONAMIDO)METHYLPHOSPHONATE

To diethyl (5,6-bis(2-chloroethoxy)benzo[d]thiazol-2-ylthioamino)methylphosphonate (0.51 g, 1.0 mmol) in dichloromethane (25 mL) at r.t. was added sodium acetate (0.85 g, 10 mmol) and m-chloroperoxybenzoic acid (0.55 g, 3.2 mmol), and the resulting mixture was stirred for 18 h, then heated to reflux for 1 h. The mixture was cooled to r.t., washed with water, saturated sodium bicarbonate, and brine, dried over magnesium sulfate, and concentrated. Column chromatography (90% ethyl acetate/hexanes) yielded the product as a colorless solid.

¹H NMR (400 MHz, CD₃OD) δ (ppm): 7.65 (s, 1H); 7.63 (s, 1H); 4.89 (br s, 1H); 4.38-4.35 (m, 4H); 4.14 (quint, J=8.2, 4H); 3.94-3.91 (m, 4H); 3.63 (d, J=11.7, 2H); 1.30 (t, J=7.0, 6H).

Utilizing the foregoing procedure, the following compound was prepared:

diethyl[5,6-bis(3-chloropropoxy)benzo[d]thiazole-2-sulfonamido]methylphosphonate

The product was purified by flash column chromatography (eluents 80% EtOAc/DCM to pure AcOEt, then EtOAc/MeOH) to give product as a pale yellow solid.

¹H NMR (400 MHz, DMSO-d₆) [symbol] ppm): 7.80 (s, 1H), 7.69 (s, 1H), 4.15-4.20 (m, 4H), 3.99 (m, 4H), 3.81 (t, J=6.5 Hz, 4H), 3.42 (d, J=11.7 Hz, 2H), 2.20 (m, 4H), 1.17 (t, J=7.0 Hz, 6H); MS m/z 547 (M−1).

### Preparative Example 35

### DIMETHYL (5-FORMYLBENZO[b]THIOPHENE-2-SULFONAMIDO)-METHYLPHOSPHONATE

To a solution of dimethyl (5-vinylbenzo[b]thiophene-2-sulfonamido)methylphosphonate (0.45 g, 1.25 mmol) in THF (75 mL) was added osmium tetroxide (4% in water, 0.1 mL), and sodium periodate (0.70 g, 3.3 mmol) in water (10 mL). This mixture was stirred for 24 h at r.t. and concentrated. The residue was extracted with ethyl acetate; the organic extract was washed with water and brine, dried over magnesium sulfate, and concentrated. Column chromatography (ethyl acetate as an eluent) provided the pure product.

¹H NMR (400 MHz, DMSO-d₆) δ (ppm): 10.10 (s, 1H); 8.74 (br s, 1H); 8.58 (d, J=1.6, 1H); 8.30 (dd, J=8.4, 0.6, 1H); 8.23 (d. J=0.6, 1H); 7.98 (dd, J=8.4, 1.6, 1H); 3.63 (d, J=10.8, 6H); 3.38 (d, J=12.1, 2H).

### Preparative Example 36

### Method A. Preparation of [({[5,6-BIS(2-AZIDOETHOXY)-1-BENZOTHIEN-2-YL]-SULFONYL}AMINO)METHYL]PHOSPHONIC ACID

A stirred mixture of phosphonic acid (110 mg, 0.24 mmol) and NaN₃ (62 mg, 4 equiv) in DMSO was heated at 80° C. for 16 h. The reaction mixture was cooled to room temperature and then purified by reverse phase HPLC to afford the product.

¹H NMR (400 MHz, CD₃OD) δ (ppm): 7.82 (s, 1H), 7.55 (s, 1H), 7.5 (s, 1H), 4.3 (m, 4H), 3.7 (m, 4H), 3.25 (d, 2H); MS m/z 478 (M+1).

Utilizing the foregoing procedure, the following compounds were prepared:

{[({5-[3,4-bis(3-azidopropoxy)phenyl]thiophen-2-yl}sulfonyl)amino]methyl}phosphonic acid was prepared and purified by reverse phase HPLC.

¹H NMR (500 MHz, CD₃OD) δ (ppm): 7.60 (d, 1H), 7.38 (d, 1H), 7.24 (m, 2H), 7.04 (d, 1H), 4.20 (m, 4H), 3.60 (m, 4H), 3.10 (d, 2H), 2.10 (m, 4H); MS m/z 532 (M+1).

{[({5-[3,4-bis(3-azidopropoxy)-2-methylphenyl]thiophen-2-yl}sulfonyl)amino]methyl}-phosphonic acid was prepared and purified by reverse phase HPLC.

¹H NMR (500 MHz, CD₃OD) δ (ppm): 7.60 (d, 1H), 7.20 (d, 1H), 7.10 (d, 1H), 7.00 (d, 1H), 4.20 (t, 2H), 4.10 (t, 2H), 3.60 (m, 4H), 3.20 (d, 2H), 2.36 (s, 3H), 2.10 (m, 4H); MS m/z 546 (M+1).

{[({5-[4,5-bis(3-azidopropoxy)-2-methylphenyl]thiophen-2-yl}sulfonyl)amino]methyl}-phosphonic acid was prepared and purified by reverse phase HPLC.

¹H NMR (500 MHz, CD₃OD) δ (ppm): 7.60 (d, 1H), 7.16 (d, 1H), 7.0 (s, 1H), 6.98 (s, 1H), 4.18 (t, 2H), 4.14 (t, 2H), 3.60 (m, 4H), 3.24 (d, 2H), 2.40 (s, 3H), 2.10 (m, 4H); MS m/z 546 (M+1).

[({[5,6-bis(3-azidopropoxy)-1-benzothien-2-yl]sulfonyl}amino)methyl]phosphonic acid

¹H NMR (500 MHz, CD₃OD) δ (ppm): 7.80 (s, 1H), 7.52 (s, 1H), 7.50 (s, 1H), 4.20 (m, 4H), 3.60 (t, 4H), 3.06 (d, 2H), 2.18 (m, 4H); MS m/z 506 (M+1).

[({[5,6-bis(4-azidobutoxy)-1-benzothien-2-yl]sulfonyl}amino)methyl]phosphonic acid

¹H NMR (500 MHz, CD₃OD) δ (ppm): 7.80 (s, 1H), 7.42 (s, 1H), 7.40 (s, 1H), 4.10 (m, 4H), 3.40 (t, 4H), 3.20 (d, 2H), 1.90 (m, 4H), 1.80 (m, 4H); MS m/z 534 (M+1).

[({[6-(4-azidoobutoxy)-5-(3-azidopropoxy)-1-benzothien-2-yl]sulfonyl}amino)methyl]-phosphonic acid and [({[6-(4-azidobutoxy)-5-(3-azidopropoxy)-1-benzothien-2-yl]-sulfonyl}amino)methyl]phosphonic acid

Major isomer (0.0639 g): ¹H NMR (500 MHz, CD₃OD) δ (ppm): 7.79 (s, 1H), 7.49 (s, 1H), 7.45 (s, 1H), 4.18 (t, 2H), 4.11 (t, 2H), 3.58 (t, 2H), 3.43 (t, 2H), 3.15 (d, 2H), 2.11 (m, 2H), 1.94 (m, 2H), 1.85 (m, 2H); MS m/z 520 (M+1).

Minor isomer (0.0206 g): ¹H NMR (500 MHz, CD₃OD) δ (ppm): 7.79 (s, 1H), 7.48 (s, 1H), 7.47 (s, 1H), 4.21 (t, 2H), 4.14 (bt, 2H), 3.83 (t, 2H), 3.71 (bt, 2H), 3.19 (d, 2H), 2.28 (m, 2H), 2.00 (m, 4H); MS m/z 520 (M+1).

[({[5,6-bis(2-azidoethoxy)-4,7-dichloro-1-benzothien-2-yl]sulfonyl}amino)methyl]phosphonic acid was prepared.

¹H NMR (400 MHz, CD₃OD) [symbol] (ppm): 8.0 (s, 1H), 4.35 (m, 4H), 3.7 (m, 4H), 3.1 (d, 2H); MS m/z 546 (M+1).

[({[5,6-bis(2-azidoethoxy)-4,7-difluoro-1-benzothien-2-yl]sulfonyl}amino)methyl]phosphonic acid

¹H NMR (400 MHz, CD₃OD) δ (ppm): 8.0 (d, 1H), 4.45 (t, 2H), 4.4 (t, 2H), 3.7 (m, 4H), 3.3 (d, 2H); MS m/z 514 (M+1).

[({[5,6-bis(2-azidoethoxy)-3-methyl-4,7-difluoro-1-benzothien-2-yl]sulfonyl}amino)methyl]-phosphonic acid

¹H NMR (500 MHz, CD₃OD) δ (ppm): 4.42 (t, 2H), 4.35 (t, 2H), 3.69 (m, 4H), 3.30 (d, 2H), 2.82 (s, 3H).

[({[5,6-bis(3-azidopropoxy)-4,7-difluoro-1-benzothien-2-yl]sulfonyl}amino)methyl]phosphonic acid

¹H NMR (500 MHz, CD₃OD) δ (ppm): 7.90 (d, 1H), 4.3 (two t, 4H), 3.6 (t, 4H), 3.1 (d, 2H); 2.1 (m, 4H); MS m/z 542 (M+1).

[({[5,6-bis(2-azidoethoxy)-3-methyl-1-benzothien-2-yl]sulfonyl}amino)methyl]phosphonic acid

¹H NMR (500 MHz, CD₃OD) δ (ppm): 7.51 (s, 1H), 7.40 (s, 1H), 4.29 (m, 4H), 3.68 (m, 4H), 3.22 (d, 2H), 2.68 (s, 3H); MS m/z 492 (M+1).

[({[5,6-bis(3-azidopropoxy)-3-methyl-1-benzothien-2-yl]sulfonyl}amino)methyl]phosphonic acid

¹H NMR (500 MHz, CD₃OD) δ (ppm): 7.5 (s, 1H), 7.4 (s, 1H), 4.2 (m, 4H), 3.6 (m, 4H), 3.25 (d, 2H), 2.65 (s, 3H), 2.15 (m, 4H); MS m/z 520 (M+1).

[({[5,6-bis(3-azidopropoxy)-3-ethyl-1-benzothien-2-yl]sulfonyl}amino)methyl]phosphonic acid

¹H NMR (500 MHz, CD₃OD) δ (ppm): 7.45 (s, 1H), 7.38 (s, 1H), 4.20 (q, 4H), 3.60 (q, 4H), 3.25 (d, 2H), 3.20 (q, 2H), 2.15 (m, 4H), 1.30 (t, 3H); MS m/z 534 (M+1).

[({[5,6-bis(3-azidopropoxy)-4,7-difluoro-3-methyl-1-benzothien-2-yl]sulfonyl}amino)methyl]-phosphonic acid

¹H NMR (500 MHz, CD₃OD) δ (ppm): 4.3 (two t, 4H), 3.6 (m, 4H), 3.3 (d, 2H); 2.8 (s, 3H), 2.1 (m, 4H); MS m/z 556 (M+1).

[({[5,6-bis(2-azidoethoxy)-4,7-difluoro-3-ethyl-1-benzothien-2-yl]sulfonyl}amino)methyl]-phosphonic acid

¹H NMR (500 MHz, CD₃OD) δ (ppm): 4.42 (t, 2H), 4.38 (t, 2H), 3.70 (m, 4H), 3.30 (d, 2H), 3.26 (m, 2H), 1.38 (t, 3H); MS m/z 452 (M+1).

[({[5,6-bis(3-azidopropoxy)-4,7-difluoro-3-ethyl-1-benzothien-2-yl]sulfonyl}amino)methyl]-phosphonic acid

¹H NMR (500 MHz, CD₃OD) δ (ppm): 4.30 (t, 2H), 4.20 (t, 2H), 3.60 (m, 4H), 3.30 (d, 2H), 3.26 (m, 2H), 2.05 (m, 4H), 1.30 (t, 3H); MS m/z 570 (M+1).

(5,6-bis(2-azidoethoxy)benzo[d]thiazole-2-sulfonamido)methylphosphonic acid was purified by reverse phase HPLC (250×21.2 mm Aquasil C18 column, 40-70% methanol/water linear gradient, 30 min. elution time; elutes ˜20 min.) to product as a white solid.

¹H NMR (400 MHz, DMSO-d₆) δ (ppm): 7.83 (s, 1H); 7.77 (s, 1H); 4.28-4.22 (m, 4H); 3.69-3.64 (m, 4H); 3.16 (d, J=13.1, 2H).

(5,6-bis(3-azidopropoxy)benzo[d]thiazole-2-sulfonamido)methylphosphonic acid

Purified by preparative HPLC (Thermo, Aquasil C18, 240×21.2 mm, 5 ≡m; eluting with a gradient MeOH/H₂O 10/90 to 95/5, both containing 0.05% HCO₂H, over 30 min) to afford title compound as a white solid. ¹H NMR (400 MHz, CD₃OD) [symbol] ppm): 7.67 (s, 1H), 7.63 (s, 1H), 4.20 (m, 4H), 3.58 (m, 4H), 3.24 (d, J=13.3 Hz, 2H), 2.12 (m, 4H); MS m/z 505 (M−1).

### Preparative Example 37

### Preparation of 4-CYANO-3-FLUOROPHENYL HYDROGEN {[({5-[3,4-BIS(3-AZIDOPROPOXY)PHENYL]THIOPHENE-2-YL}SULFONYL)AMINO}METHYL}PHOSPHONATE

A mixture of the phosphonic acid (0.2 g, 0.38 mmol), 2-fluoro-4-hydroxybenzonitrile (0.077 g, 1.5 equiv), and trichloroacetonitrile (380 uL, 10 equiv) in a solvent mixture of 2.0 mL of anhydrous pyridine and 0.2 mL of DMF in a sealed tube was heated at 105° C. for 6 h. The reaction mixture was concentrated and used as is in the next step.

MS m/z 623 (M−28).

Utilizing the foregoing procedure the follow compounds were prepared:

4-cyano-3-fluorophenyl hydrogen {[({5-[3,4-bis(3-azidopropoxy)-2-methylphenyl]thiophen-2-yl}sulfonyl)amino]methyl}phosphonate was prepared and used as is in the next step.

MS m/z 637 (M−28).

4-cyano-3-fluorophenyl hydrogen {[({5-[4,5-bis(3-azidopropoxy)-2-methylphenyl]thiophen-2-yl}sulfonyl)amino]methyl}phosphonate was prepared and used as is in the next step.

MS m/z 637 (M−28).

4-cyano-3-fluorophenyl hydrogen[({[5,6-bis(3-azidopropoxy)-1-benzothien-2-yl]sulfonyl}-amino)methyl]phosphonate was prepared and purified by reverse phase HPLC.

¹H NMR (500 MHz, CD₃OD) δ (ppm): 7.79 (s, 1H), 7.60 (m, 1H), 7.44 (s, 1H), 7.40 (s, 1H), 7.15 (m, 2H), 4.20 (m, 4H), 3.60 (m, 4H), 3.44 (d, 2H), 2.16 (m, 4H); MS m/z 597 (M−27).

4-cyano-3-fluorophenyl hydrogen[({[5,6-bis(3-azidopropoxy)-3-ethyl-1-benzothien-2-yl]-sulfonyl}amino)methyl]phosphonate

MS m/z 625 (M−27).

4-cyano-3-fluorophenyl hydrogen[({[4,7-difluoro-5,6-bis(2-azidoethoxy)-3-ethyl-1-benzothien-2-yl]sulfonyl}amino)methyl]phosphonate

MS m/z 633 (M−27).

4-cyano-3-fluorophenyl hydrogen[({[4,7-difluoro-5,6-bis(3-azidopropoxy)-3-ethyl-1-benzothien-2-yl]sulfonyl}amino)methyl]phosphonate

MS m/z 661 (M−27).

4-cyano-3-fluorophenyl hydrogen[({[5,6-bis(4-azidobutoxy)-1-benzothien-2-yl]sulfonyl}-amino)methyl]phosphonate was prepared and used as is in the next step.

MS m/z 625 (M+1).

4-cyano-3-fluorophenyl hydrogen[({[6-(4-azidobutoxy)-5-(3-azido propoxy)-1-benzothien-2-yl]sulfonyl}amino)methyl]phosphonate and 4-cyano-3-fluorophenyl hydrogen[({[6-(4-azidobutoxy)-5-(3-azidopropoxy)-1-benzothien-2-yl]sulfonyl}amino)methyl]phosphonate

The products were obtained after purification by silica gel plate chromatography (1000 micron, 40/10/1 CHCl₃/MeOH/conc. NH₄OH).

Major isomer (0.0263 g): ¹H NMR (500 MHz, CD₃OD) δ (ppm): 7.71 (s, 1H), 7.43 (s, 1H), 7.43 (t, 1H), 7.37 (s, 1H), 7.10 (dd, 1H), 7.03 (dd, 1H), 4.20 (t, 2H), 4.11 (t, 2H), 3.59 (t, 2H), 3.44 (t, 2H), 3.26 (d, 2H), 2.11 (m, 2H), 1.94 (m, 2H), 1.85 (m, 2H); MS m/z 639 (M+1).

Minor isomer (0.0088 g): ¹H NMR (500 MHz, CD₃OD) δ (ppm): 7.72 (s, 1H), 7.43 (s, 1H), 7.43 (t, 1H), 7.40 (s, 1H), 7.10 (dd, 1H), 7.03 (dd, 1H), 4.16 (m, 4H), 3.59 (t, 2H), 3.45 (t, 2H), 3.25 (d, 2H), 2.11 (m, 2H), 1.95 (m, 2H), 1.85 (m, 2H).

4-cyano-3-fluorophenyl hydrogen (5,6-bis(2-azidoethoxy)benzo[d]thiazole-2-sulfonamido)methylphosphonate

The product was partially purified by reverse phase HPLC (250×21.2 mm Aquasil C18 column, 45%-90% methanol/water linear gradient, 30 min. elution time; elutes ˜15 min.) and was contaminated with large amounts of un-reacted 2-fluoro-4-hydroxybenzonitrile. It was used as is for the next transformation.

4-cyano-3-fluorophenyl hydrogen (5,6-bis(2-azidopropoxy)benzo[d]thiazole-2-sulfonamido)methylphosphonate

Purification by flash chromatography on silica gel (MeOH/NH₄OH/CHCl₃: 20/1/79) to afforded the title compound as a pale yellow solid.

MS m/z 624 (M−1).

Utilizing the foregoing procedure and substituting 2-trifluoromethyl-4-hydroxybenzonitrile for 2-fluoro-4-hydroxybenzonitrile, the following compound was prepared:

4-cyano-3-(trifluoromethy)lphenyl hydrogen[({[5,6-bis-(3-azidopropoxy)-1-benzothien-2-yl]-sulfonyl}amino)methyl]phosphonate

MS m/z 647 (M⁺−27).

Utilizing the foregoing procedure and 2-fluoro-4-hydroxyphenyl-trifluoromethylsulfone, as the coupling partner, the following compounds were prepared:

3-fluoro-4-[(trifluoromethyl)sulfonyl]phenyl hydrogen[({[5,6-bis(2-azidoethoxy)-4,7-dichloro-1-benzothien-2-yl]sulfonyl}amino)methyl]phosphonate

MS m/z 744 (M⁺−28).

3-fluoro-4-[(trifluoromethyl)sulfonyl]phenyl hydrogen[({[5,6-bis(2-azidoethoxy)-1-benzothien-2-yl]sulfonyl}amino)methyl]phosphonate was prepared and used as is in the next step.

Utilizing the foregoing procedure and 2-cyano-5-hydroxy-pyridine as the coupling partner, the following compound was prepared:

6-cyanopyridin-3-yl hydrogen[({[5,6-bis(2-azidoethoxy)-1-benzothien-2-yl]sulfonyl}-amino)methyl]phosphonate

¹H NMR (500 MHz, CD₃OD) [symbol] ppm: 8.50 (s, 1H), 7.80 (m, 2H), 7.46 (m 3H), 4.26 (t, 4H), 3.64 (t, 4H), 3.25 (d, 2H); MS m/z 579 (M⁺).

### Preparative Example 38

### Preparation of 2-[({[(4-CYANO-3-FLUOROPHENOXY)(HYDROXY)PHOSPHORYL]-METHYL}AMINO)SULFONYL]-1-BENZOTHIOPHENE-5-CARBOXYLIC ACID

A solution of ({[(5-formyl-1-benzothien-2-yl)sulfonyl]amino}methyl)phosphonic acid (0.22 g, 0.66 mmol), trichloroacetonitrile (1.0 mL, 10 mmol) and 2-fluoro-4-hydroxybenzonitrile (0.18 g, 1.3 mmol) in anhydrous pyridine (10 mL) was heated at 120° C. for 2.5 h in a pressure bottle. The reaction mixture was then concentrated and the residue triturated with ether and ethyl acetate, and dissolved in 50% aqueous acetone (25 mL). To this solution were added 2-methyl-2-butene (5 mL), sodium chlorite (0.020 g, 0.22 mmol) and potassium dihydrogen phosphate (0.020 g, 0.15 mmol) in water (1 mL). The mixture was stirred for 30 min then concentrated. The residue was purified by reverse phase HPLC (250×21.2 mm Aquasil C18 column, 30%-70% methanol/water linear gradient, 30 min elution time, elutes ˜16 min) to yield the product as an amorphous white solid.

¹H NMR (400 MHz, CD₃OD) δ (ppm): 8.50 (s, 1H); 8.02 (dd, J=8.4, 1.4, 1H); 7.92 (d, J=8.8, 1H); 7.91 (s, 1H); 7.39 (t, J=8.2, 1H); 7.04-6.96 (m, 2H). [CH₂ peak hidden underneath residual methanol peak at ˜3.2 ppm].

### Preparative Example 39

### Preparation of 4-CYANO-3-FLUOROPHENYL [({[5,6-BIS(2-AMMONIOETHOXY)-1-BENZOTHIEN-2-YL]SULFONYL}AMINO)METHYL]PHOSPHONATE TRIFLUOROACETATE

A mixture consisting of [({[5,6-bis(2-azidoethoxy)-1-benzothien-2-yl]sulfonyl}amino)methyl]-phosphonic acid (187 mg, 0.39 mmol), the phenol (70 mg, 1.3 equiv), trichloroacetonitrile (0.4 mL, 10 equiv), anhydrous pyridine (3 mL) and 10% of DMF (0.3 mL) was stirred in a sealed tube for 6 h at 105° C. After the reaction was complete, pyridine was evaporated in vacuum to give an oily residue. To a solution of the crude adduct in MeOH was added palladium black (19 mg, 10%). The resulting mixture was stirred under 40 psi of hydrogen gas overnight. The resulting dark yellow solution was filtered and the organic layer was evaporated to give an oil, which was purified by reverse phase HPLC to afford the diamine.

¹H NMR (400 MHz, CD₃OD) δ (ppm): 7.8 (s, 1H), 7.6 (brt, 1H), 7.45 (s, 1H), 7.4 (s, 1H), 7.2 (m, 2H), 4.3 (m, 4H), 3.7 (m, 4H), 3.5 (d, 2H); MS m/z 545 (M+1).

Utilizing the foregoing procedure, the following compounds were prepared:

4-cyano-3-fluorophenyl hydrogen (5,6-bis(2-aminoethoxy)benzo[d]thiazole-2-sulfonamido)-methylphosphonate

Purified by reverse phase HPLC (250×21.2 mm Aquasil C18 column, 20%-60% methanol/water linear gradient, 30 min. elution time; elutes ˜15 min.) to give the product over two steps as a colorless solid.

¹H NMR (400 MHz, CD₃OD) δ (ppm): 7.69 (s, 1H); 7.63 (s, 1H); 7.42 (t, J=8.6, 1H); 7.08-7.04 (m, 2H); 4.63 (br s, 4H); 4.40-4.36 (m, 4H); 4.45 (d, J=13.1, 2H); MS m/z 546 (M).

4-cyano-3-fluorophenyl hydrogen (5,6-bis(2-ammoniopropoxy)benzo[d]thiazole-2-sulfonamido)methylphosphonate trifluoroacetate

The residue was dissolved in a few drops of TFA, diluted with MeOH (2 mL) and purified by preparative HPLC (Thermo, Aquasil C18, 240×21.2 mm, 5 μm; gradient MeOH/H₂O: 10/90 to 95/5, both containing 0.05% HCO₂H over 30 min) twice to afford the title compound as a white solid.

¹H NMR (400 MHz, CD₃OD) [symbol] ppm): 8.07 (s, 1H), 7.47 (s, 7.42(s, 1H), 7.35 (dd, J=8.0, 8.4 Hz, 1H), 7.02 (m, 2H), 4.24 (m, 4H), 3.46 (d, J=12.7 Hz, 2H), 3.25 (m, 4H), 2.26 (m, 4H); MS m/z 572 (M−1).

4-cyano-3-fluorophenyl hydrogen ({[(5-amino-1-benzothien-2-yl)sulfonyl]amino}methyl)-phosphonate prepared as a pale pink solid.

¹H NMR (400 MHz, DMSO-d₆) δ (ppm): 7.99-7.91 (m, 1H), 7.82-7.76 (m, 2H), 7.72 (dd, J=8.3 Hz, 1H), 7.32-7.25 (m, 2H), 7.09 (dd, J=8.4, 2.0 Hz, 1H), 7.03 (d, J=8.4 Hz, 1H), 3.04 (dd, J=12.5, 5.9 Hz, 2H).

4-cyano-3-(trifluoromethyl)phenyl[({[5,6-bis(2-ammonioethoxy)-1-benzothien-2-yl]-sulfonyl}amino)methyl]phosphonate trifluoroacetate

¹H NMR (400 MHz, DMSO-d₆) δ (ppm): 7.9 (d, 1H), 7.8 (d, 1H), 7.75 (s, 1H), 7.7 (s, 1H), 7.55 (s, 1H), 7.5 (dd, 1H), 4.2 (m, 4H), 3.3 (m, 4H), 2.9 (d, 2H); MS m/z 595 (M+1).

3-fluoro-4-[(trifluoromethyl)sulfonyl]phenyl-[({[5,6-bis(2-ammonioethoxy)-1-benzothien-2-yl]-sulfonyl}amino)methyl]phosphonate trifluoroacetate

¹H NMR (500 MHz, CD₃OD) δ (ppm): 7.75 (s, 1H), 7.70 (t, 1H), 7.55 (s, 1H), 7.46 (s 1H), 7.19 (m, 2H), 4.36 (t, 4H), 3.44 (t, 4H), 3.25 (d, 2H); MS m/z 652 (M+1).

6-cyanopyridin-2-yl-[({[5,6-bis(2-ammonioethoxy)-1-benzothien-2-yl]sulfonyl}amino)-methyl]-phosphonate trifluoroacetate

¹H NMR (500 MHz, CD₃OD) δ (ppm): 8.30 (d, 1H), 7.70 (m, 2H), 7.60 (d, 1H), 7.56 (s 1H), 7.46 (s, 1H), 4.40 (m, 4H), 3.44 (m, 4H), 3.25 (d, 2H); MS m/z 528 (M+1).

4-cyano-3-fluorophenyl[({[5,6-bis(2-ammonioethoxy)-3-methyl-1-benzothien-2-yl]sulfonyl}-amino)methyl]phosphonate trifluoroacetate

¹H NMR (500 MHz, CD₃OP) δ (ppm): 7.50 (s, 1H), 7.33 (s, 1H), 7.31 (t, 1H), 6.94 (m, 2H), 4.39 (m, 4H), 3.49 (t, 4H), 3.28 (d, 2H), 2.56 (s, 3H); MS m/z 559 (M+1).

4-cyano-3-fluorophenyl[({[5,6-bis(2-ammonioethoxy)-3-ethyl-1-benzothien-2-yl]sulfonyl}-amino)methyl]phosphonate trifluoroacetate

¹H NMR (500 MHz, CD₃OD) δ (ppm): 7.54 (s, 1H), 7.38 (s, 1H), 7.36 (m, 1H), 6.98 (m, 2H), 4.20 (m, 4H), 3.50 (m, 4H), 3.30 (d, 2H), 3.10 (q, 2H), 1.25 (t, 3H); MS m/z 573 (M+1).

4-cyano-3-fluorophenyl[({[5,6-bis(3-ammoniopropoxy)-1-benzothien-2-yl]sulfonyl}amino)-methyl]phosphonate trifluoroacetate

¹H NMR (500 MHz, CD₃OD) δ (ppm): 7.62 (s, 1H), 7.40 (s, 1H), 7.32 (s, 1H), 7.30 (m, 1H), 7.00 (m, 2H), 4.24 (m, 4H), 3.30 (d, 2H), 3.2 (m, 4H), 2.24 (m, 4H); MS m/z 573 (M+1).

4-cyano-3-fluorophenyl hydrogen[({[5,6-bis(3-ammoniopropoxy)-3-ethyl-1-benzothien-2-yl]-sulfonyl}amino)methyl]phosphonate trifluoroacetate

¹H NMR (500 MHz, CD₃OD) δ (ppm): 7.40 (s, 1H), 7.26 (m, 1H), 7.20 (s, 1H), 6.90 (m, 2H), 4.26 (m, 4H), 3.30 (d, 2H), 3.26 (m, 4H), 3.05 (d, 2H), 3.20 (q, 2H), 2.25 (m, 4H), 1.25 (t, 3H); MS m/z 601 (M+1).

4-cyano-3-fluorophenyl[({[5,6-bis(4-ammoniobutoxy)-1-benzothien-2-yl]sulfonyl}amino)-methyl]phosphonate trifluoroacetate

¹H NMR (500 MHz, CD₃OD) δ (ppm): 7.70 (s, 1H), 7.42 (s, 1H), 7.40 (m, 2H), 7.00 (m, 2H), 4.20 (m, 4H), 3.30 (d, 2H), 3.15 (m, 4H), 2.00 (m, 8H); MS m/z 601 (M+1).

4-cyano-3-fluorophenyl hydrogen[({[6-(4-ammoniobutoxy)-5-(3-ammoniopropoxy)-1-benzothien-2-yl]sulfonyl}amino)methyl]phosphonate and 4-cyano-3-fluorophenyl hydrogen[({[5-(4-ammoniobutoxy)-6-(3-ammoniopropoxy)-1-benzothien-2-yl]sulfonyl}amino)methyl]phosphonate.

Major isomer: ¹H NMR (500 MHz, CD₃OD) δ (ppm): 7.62 (s, 1H), 7.38 (s, 1H), 7.32 (t, 1H), 7.32 (s, 1H), 7.01 (m, 2H), 4.28 (t, 2H), 4.12 (t, 2H), 3.26 (m, 4H), 3.07 (t, 2H), 2.26 (m, 2H), 1.97 (m, 2H), 1.91 (m, 2H).

Minor isomer: ¹H NMR (500 MHz, CD₃OD) δ (ppm): 7.65 (s, 1H), 7.44 (s, 1H), 7.37 (s, 1H), 7.33 (t, 1H), 7.00 (m, 2H), 4.26 (t, 2H), 4.19 (t, 2H), 3.27 (t, 2H), 3.25 (d, 2H), 3.07 (t, 2H), 2.24 (m, 2H), 1.99 (m, 2H), 1.90 (m, 2H).

4-cyano-3-(trifluoromethyl)phenyl[({[5,6-bis(3-ammoniopropoxy)-1-benzothien-2-yl]sulfonyl}-amino)methyl]phosphonate trifluoroacetate

¹H NMR (500 MHz, CD₃OD) δ (ppm): 7.62 (m, 3H), 7.40 (m, 2H), 7.38 (s, 1H), 4.24 (m, 4H), 3.32 (d, 2H), 3.26 (m, 4H), 2.26 (m, 4H); MS m/z 623 (M+1).

4-cyano-3-fluorophenyl[({[5,6-bis(2-ammonioethoxy)-4,7-dichloro-1-benzothien-2-yl]sulfonyl}-amino)methyl]phosphonate trifluoroacetate

¹H NMR (400 MHz, CD₃OD) δ (ppm): 7.9 (s, 1H), 7.4 (t, 1H), 7.05 (d, 1H), 7.0 (d, 1H), 4.4 (m, 4H), 3.42 (m, 4H), 3.3 (d, 2H); MS m/z 613 (M+1).

4-cyano-3-(trifluoromethyl)phenyl[({[5,6-bis(2-ammonioethoxy)-4,7-dichloro-1-benzothien-2-yl]sulfonyl}amino)methyl]phosphonate trifluoroacetate

¹H NMR (400 MHz, CD₃OD) δ (ppm): 7.9 (s, 1H), 7.8 (d, 1H), 7.65 (s, 1H), 7.5 (d, 1H), 4.4 (m, 4H), 3.42 (m, 4H), 3.3 (d, 2H); MS m/z 663 (M+1).

3-fluoro-4-[(trifluoromethyl)sulfonyl]phenyl-[({[5,6-bis(2-ammonioethoxy)-4,7-dichloro-1-benzothien-2-yl]sulfonyl}amino)methyl]phosphonate trifluoroacetate

¹H NMR (500 MHz, CD₃OD) δ (ppm): 8.00 (s, 1H), 7.85 (m, 1H), 7.40 (m, 1H), 7.25 (m, 1H), 4.40 (t, 4H), 3.50 (t, 4H), 3.35 (d, 2H); MS m/z 720 (M).

4-cyano-3-fluorophenyl[({[5,6-bis(2-ammonioethoxy)-4,7-difluoro-1-benzothien-2-yl]sulfonyl}-amino)methyl]phosphonate trifluoroacetate

¹H NMR (400 MHz, CD₃OD) δ (ppm): 7.85 (brs, 1H), 7.5 (brt, 1H), 7.1 (m, 1H), 4.5 (two brd, 4H), 3.4 (m, 4H), 3.3 (d, 2H, hidden); MS m/z 581 (M+1).

4-cyano-3-(trifluoromethyl)phenyl[({[5,6-bis(2-ammonioethoxy)-4,7-difluoro-1-benzothien-2-yl]sulfonyl}amino)methyl]phosphonate trifluoroacetate

¹H NMR (400 MHz, CD₃OD) δ (ppm): 7.9 (d, 1H), 7.8 (d, 1H), 7.75 (brs, 1H), 7.55 (d, 1H), 4.55 (t, 2H), 4.45 (t, 2H), 3.3 (d, 2H, hidden); MS m/z 631 (M+1).

4-cyano-3-fluorophenyl[({[5,6-bis(2-ammonioethoxy)-3-methyl-4,7-difluoro-1-benzothien-2-yl]sulfonyl}amino)methyl]phosphonate trifluoroacetate

¹H NMR (500 MHz, CD₃OD) δ (ppm): 7.49 (t, 1H), 6.99-7.08 (m, 2H), 4.50 (t, 2H), 4.41 (t, 2H), 3.42 (m, 4H), 3.32 (d, 2H), 2.72 (s, 3H); MS m/z 595 (M+1).

4-cyano-3-fluorophenyl[({[5,6-bis(2-ammonioethoxy)-3-ethyl-4,7-difluoro-1-benzothien-2-yl]sulfonyl}amino)methyl]phosphonate trifluoroacetate

¹H NMR (500 MHz, CD₃OD) δ (ppm): 7.50 (t, 1H), 7.05 (d, 1H), 7.00 (d, 1H), 4.50 (t, 2H), 4.40 (t, 2H), 3.40 (m, 4H), 3.30 (d, 2H), 3.20 (q, 2H), 1.25 (t, 3H); MS m/z 609 (M+1).

4-cyano-3-(trifluoromethyl)phenyl[({[5,6-bis(3-ammoniopropoxy)-4,7-difluoro-1-benzothien-2-yl]sulfonyl}amino)methyl]phosphonate trifluoroacetate

¹H NMR (500 MHz, CD₃OD) δ (ppm): 7.8 (brs, 1H), 7.5 (t, 1H), 7.1 (m, 2H), 4.4 (two t, 4H), 3.3 (d, 2H, hidden), 3.25 (m, 4H), 2.2 (m, 4H); MS m/z 609 (M+1).

4-cyano-3-fluorophenyl[({[5,6-bis(3-ammoniopropoxy)-3-methyl-1-benzothien-2-yl]-sulfonyl}amino)methyl]phosphonate trifluoroacetate

¹H NMR (500 MHz, CD₃OD) δ (ppm): 7.4 (s, 1H), 7.3 (t, 1H), 7.2 (s, 1H), 6.92 (s, 1H), 6.9 (dd, 1H), 4.3 (m, 4H), 2.5 (s, 3H), 2.3 (m, 4H); MS m/z 587 (M+1).

4-cyano-3-fluorophenyl[({[5,6-bis(3-ammoniopropoxy)-4,7-difluoro-3-methyl-1-benzothien-2-yl]sulfonyl}amino)methyl]phosphonate trifluoroacetate

¹H NMR (500 MHz, CD₃OD) δ (ppm): 7.45 (t, 1H), 7.15 (dd, 1H), 6.95 (d, 1H), 4.4 (two t, 4H), 3.53 (d, 2H, hidden), 3.25 (m, 4H), 2.7 (s, 3H), 2.2 (m, 4H); MS m/z 623 (M+1).

4-cyano-3-fluorophenyl[({[5,6-bis(3-ammoniopropoxy)-4,7-difluoro-3-ethyl-1-benzothien-2-yl]-sulfonyl}amino)methyl]phosphonate trifluoroacetate

¹H NMR (500 MHz, CD₃OD) δ (ppm): 7.50 (t, 1H), 7.05 (d, 1H), 7.00 (d, 1H), 4.42 (t, 2H), 4.38 (t, 2H), 3.40 (d, 2H), 3.26 (m, 4H), 3.20 (q, 2H), 2.20 (m, 4H), 1.25 (t, 3H); MS m/z 637 (M+1).

4-cyano-3-fluorophenyl-{[({5-[3,4-bis(3-ammoniopropoxy)phenyl]thiophen-2-yl}sulfonyl)-amino]methyl}phosphonate trifluoroacetate

¹H NMR (500 MHz, CD₃OD) δ (ppm): 7.50 (m, 2H), 7.24 (m, 3H), 7.10 (m, 3H), 4.20 (m, 4H), 3.20 (m, 6H), 2.20 (m, 4H); MS m/z 598 (M+1).

4-cyano-3-fluorophenyl-{[({5-[3,4-bis(3-ammoniopropoxy)-2-methylphenyl]thiophen-2-yl}sulfonyl)amino]methyl}phosphonate trifluoroacetate

¹H NMR (500 MHz, CD₃OD) δ (ppm): 7.60 (t, 1H), 7.52 (d, 1H), 7.20 (m, 2H), 7.08 (d, 1H), 6.98 (d, 1H), 6.96 (d, 1H), 4.20 (t, 2H), 4.06 (t, 2H), 3.20 (m, 6H), 2.22 (s, 3H), 2.20 (m, 4H); MS m/z 613 (M+1).

4-cyano-3-fluorophenyl-{[({5-[4,5-bis(3-ammoniopropoxy)-2-methylphenyl]thiophen-2-yl}-sulfonyl)amino]methyl}phosphonate

¹H NMR (500 MHz, CD₃OD) δ (ppm): 7.60 (m, 2H), 7.20 (m, 2H), 7.10 (d, 1H), 7.00 (d, 2H), 4.22 (t, 2H), 4.20 (t, 2H), 3.22 (d, 2H), 3.20 (m, 4H), 2.38 (s, 3H), 2.20 (m, 4H); MS m/z 613 (M+1).

### Preparative Example 40

### Preparation of 3,4-BIS {3-[3,4-BIS(METHOXYMETHOXY)BENZAMIDO]PROPOXY}BENZOIC ACID

Step 1. Ethyl 3,4-bis(methoxymethoxy)benzoate

To a stirred solution of ethyl 3,4-dihydroxybenzoate (2.00 g, 10.98 mmol) and NaI (cat) in anhydrous DMF (20 mL) at 0° C. under argon were added drop wise diisopropylethylamine (6.7 mL, 38.42 mmol) and chloromethyl methyl ether (4.0 mL, 52.66 mmol). The reaction mixture was allowed to warm to room temperature and stirred for two days. It was diluted with AcOEt, and washed with sat. NaHCO₃ solution, sat. NH₄Cl, H₂O and brine, dried over Na₂SO₄, filtered, concentrated and dried under high vacuum to afford the title compound (2.93 g, 10.84 mmol, 98% yield) as a dark yellow oil.

¹H NMR (400 MHz, CDCl₃) δ (ppm): ABX system (δ_{A}=7.19, δ_{B}=7.71, δ_{X}=7.82, J_{AB}=8.4 Hz, J_{BX}=2.0 Hz, J_{AX}=0 Hz, 3H), 5.31 and 5.29 (2s, 2x2H), 4.36 (q, J=7.1 Hz, 2H), 3.54 and 3.52 (2s, 2x3H), 1.39 (t, J=7.1 Hz, 3H).

Step 2. Sodium 3,4-bis(methoxymethoxy)benzoate

To a stirred solution of the product from the prior step (2.93 g) in methanol (40 mL) at room temperature was added an aqueous solution of 1N NaOH (21.7 mL). After stirring for 2.5 days, the reaction mixture was concentrated under reduced pressure, dissolved in a minimum of methanol and precipitated with AcOEt. The suspension was shaken for 1 h, the solid material was collected by filtration, rinsed with AcOEt, and dried under high vacuum to afford the title compound (3.91 g) as a white solid.

¹H NMR (400 MHz, DMSO-d₆) δ (ppm): ABX system (δ_{A}=6.99, δ_{B}=7.49, δ_{X}=7.64, J_{AB}=8.4 Hz, J_{BX}=2.0 Hz, J_{AX}=0 Hz, 3H), 5.20 and 5.18 (2s, 2x2H), 3.44 and 3.43 (2s, 2x3H).

Step 3. 3,4-Bis(methoxymethoxy)benzoic acid

To a stirred solution of the product from the prior step (1.62 g) in methanol (3 mL) and water (100 mL) at room temperature was slowly added an aqueous solution of 1N HCl in order to reach pH 4-5. After shaking for 1 h, the suspension was filtered; the precipitate was rinsed with water, air-dried and dried under high vacuum to afford the title compound (704 mg, 2.90 mmol, 47% over 2 steps) as a white fluffy solid.

¹H NMR (400 MHz, DMSO-d₆) δ (ppm): 12.83 (bs, 1H), ABX system (δ_{A}=7.22, δ_{B}=7.62, δ_{X}=7.68, J_{AB}=8.5 Hz, J_{BX}=2.1 Hz, J_{AX}=0 Hz, 3H), 5.32 (s, 2H), 5.26 (s, 2H), 3.45 and 3.44 (2s, 2x3H).

Step 4. Ethyl 3,4-bis(3-chloropropoxy)benzoate

To a stirred solution of ethyl 3,4-dihydroxybenzoate (2.00 g, 10.98 mmol) in acetone (100 mL) at room temperature were added 1-bromo-3-choropropane (3.63 g, 23.05 mmol) and potassium carbonate (6.07 g, 43.91 mmol). The reaction mixture was refluxed for 24 h, cooled to room temperature and filtered. The filtrate was collected, concentrated under reduced pressure, and purified by flash chromatography on silica gel (eluent AcOEt/hexane: 10/90→25/75) to afford the title compound (2.90 g, crude) as a yellow sticky solid which was used as is.

Step 5. Ethyl 3,4-bis(3-azidopropoxy)benzoate

To a stirred solution of the product from the prior step (2.90 g, 8.65 mmol) in DMSO (20 mL) at room temperature was added NaN₃ (2.81 g, 43.26 mmol). The reaction mixture was heated at 70° C. for 18 h, cooled to room temperature, diluted with AcOEt, and washed with water (x4), sat. NaHCO₃, water and concentrated under reduced pressure. The residue was purified by flash chromatography on silica gel (eluent AcOEt/hexane: 10/90→25/75) to afford the title compound (2.02 g, 5.80 mmol, 53% over 2 steps) as a yellow oil.

¹H NMR (400 MHz, CDCl₃) δ (ppm): ABX system (δ_{A}=6.90, δ_{B}=7.69, δ_{X}=7.57, J_{AB}=8.5 Hz, J_{BX}=2.0 Hz, J_{AX}=0 Hz, 3H), 4.36 (q, J=7.1 Hz, 2H), 4.15 (t, J=6.0 Hz, 4H), 3.56 (t, J=6.6 Hz, 2H), 3.557 (t, J=6.6 Hz, 2H), 2.12 (quin, J=6.3 Hz, 2H), 2.11 (quin, J=6.3 Hz, 2H), 1.40 (t, J=7.1 Hz, 3H).

Step 6. Ethyl 3,4-bis(3-aminopropoxy)benzoate

To a stirred solution of the product from the prior step (1.50 g, 4.31 mmol) in methanol (100 mL) at room temperature under nitrogen was added palladium black (46 mg, 0.43 mmol), and the mixture was stirred under an atmosphere of hydrogen for 2 h. The reaction mixture was filtered through paper and cotton, concentrated under reduced pressure, and dried under high vacuum to afford the title compound (1.28 g, crude) as a pale yellow sticky oil.

MS m/z 297 (M+1).

Step 7. Ethyl 3,4-bis {3-[3,4-bis(methoxymethoxy)benzamido]propoxy}benzoate

To a stirred solution of 3,4-bis(methoxymethoxy)benzoic acid (446 mg, 1.84 mmol) in anhydrous DMF (10 mL) at room temperature under nitrogen were added Et₃N (367 μL, 2.63 mmol) and benzotriazol-1-yloxytris(dimethylamino)phosphonium hexfluorophosphate (BOP) (970 mg, 2.19 mmol). After 20 min, a solution of ethyl 3,4-bis(3-aminopropoxy)benzoate (260 mg, 0.88 mmol) and Et₃N (367 μL, 2.91 mmol) in anhydrous DMF (5 mL) was added drop wise. The reaction mixture was stirred for 3.5 h, poured into water and diluted with AcOEt. The organic layer was separated, washed with sat. NaHCO₃, water, sat. NH₄Cl, water and brine, dried over Na₂SO₄, filtered, and concentrated. The crude mixture was purified twice by flash chromatography on silica gel (eluent MeOH/CH₂Cl₂: 2/98→5/95) and (eluent AcOEt/CH₂Cl₂: 50/50→MeOH/CH₂Cl₂: 5/95) to afford the title compound (480 mg, 0.64 mmol, 73% yield) as a colorless sticky solid.

¹H NMR (400 MHz, DMSO-d₆) δ (ppm): 8.46 (t, J=5.2 Hz, 2H), ABX system (δ_{A}=7.13, δ_{B}=7.49, δ_{X}=7.59, J_{AB}=8.6 Hz, J_{BX}=2.0 Hz, J_{Ax}=0 Hz, 6H), A′B′X′ system (δ_{A′}=7.08, δ_{B′}=7.57, δ_{X′}=7.46, J_{A′B′}=8.6 Hz, J_{B′X′}=2.0 Hz, J_{A′X′}=0 Hz, 3H), 5.24 (s, 4H), 5.21 (s, 4H), 4.26 (q, J=7.0 Hz, 2H), 4.07 (t, J=6.6 Hz, 4H), 3.46-3.38 (m, 4H), 3.40 and 3.39 (2s, 2x6H), 2.04-1.94 (m, 4H), 1.29 (t, J=7.1 Hz, 3H).

### Step 8.

To a stirred solution of the product of the prior step (475 mg, 0.64 mmol) in MeOH (30 mL) at room temperature was added an aqueous solution of 1N NaOH (3.6 mL, 3.60 mmol). The reaction mixture was heated at 70° C. overnight, cooled to room temperature, concentrated under reduced pressure and diluted with water. The pH was adjusted to 5 with 1N HCl and the resulting suspension was extracted with CH₂Cl₂. The combined extracts were concentrated, and the sticky residue was washed with hexane and dried under high vacuum to afford the final product, 3,4-bis{3-[3,4-bis(methoxymethoxy)benzamido]-propoxy}benzoic acid (410 mg, 0.57 mmol, 90% yield) as an off-white sticky solid.

¹H NMR (400 MHz, DMSO-d₆) δ (ppm): 8.46 (t, J=5.5 Hz, 2H), ABX system (δ_{A}=7.13, δ_{B}=7.49, δ_{X}=7.59, J_{AB}=8.4 Hz, J_{BX}=2.0 Hz, J_{AX}=0 Hz, 6H), A′B′X′ system (δ_{A′}=7.05, δ_{B′}=7.54, δ_{X′}=7.46, J_{A′B′}=8.5 Hz, J_{B′X′}=2.0 Hz, J_{A′X′}=0 Hz, 3H), 5.24 (s, 4H), 5.21 (s, 4H), 4.14-4.02 (m, 4H), 3.46-3.37 (m, 4H), 3.40 and 3.39 (2s, 2x6H), 2.05-1.93 (m, 4H).

### Preparative Example 41

### Preparation of [3,4-BIS(ACEYLOXY)PHENYL]ACETIC ACID

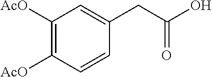

To a solution of 3,4-dihydroxy-phenyl acetic acid (1.0 g, 5.95 mmols) in Ac₂O (5.0 mL) at r.t., was added conc. H₂SO₄ (0.1 mL). The mixture was stirred at r.t., for 18 hrs. The reaction mixture was partitioned between EtOAc and D.I. H₂O and washed with brine. The extract was dried over Na₂SO₄ and filtered.

The filtrate was concentrated in vacuo. The crude mixture was purified using silica gel chromatography (10% MeOH-DCM) to give 160 mg of product.

¹H NMR (500 MHz, d₆-acetone) δ (ppm): 7.2 (m, 3H), 3.6 (s, 2H), 2.2 (d, 6H).

### Example 1

### Preparation of {2-[({[[4-cyano-3-(trifluoromethyl)phenoxy](hydroxy)phosphoryl]methyl}amino)sulfonyl]-4,7-difluoro-1-benzothiene-5,6-diyl}bis(oxyethane-2,1-diyliminocarbonylbenzene-4,1,2-triyl) tetraacetate

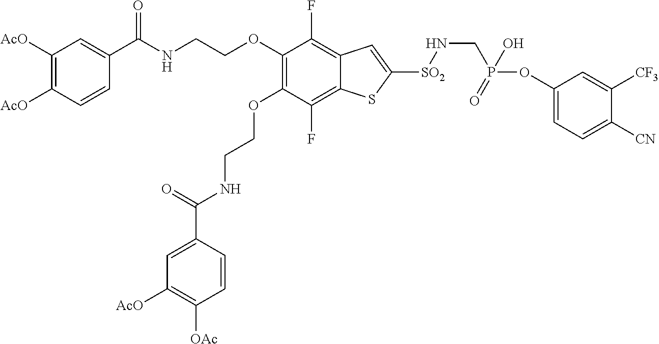

To a stirred solution of 4-cyano-3-(trifluoromethyl)phenyl[({[5,6-bis(2-ammonioethoxy)-4,7-difluoro-1-benzothien-2-yl]sulfonyl}amino)methyl]phosphonate trifluoroacetate (53 mg, 0.084 mmol) and benzoic acid (60 mg, 3 equiv.) in dry DMF (1 mL) at 0° C. under nitrogen was added O-(7-azabenzotriazol-1-yl)-N,N,N′,N′, tetramethyluronium hexafluorophosphate (HATU) (96 mg, 3 equiv.), followed by diisopropylethyl amine (0.073 mL, 5 equiv.). The cooling bath was then removed and the mixture stirred for 1.5 h at room temperature. The reaction mixture was quenched with a couple drops of HOAc and then purified by HPLC using a reverse phase column to afford the product (40 mg, 44%).

¹H NMR (600 MHz, CD₃OD) δ (ppm): 7.82 (m, 2H), 7.7-7.64 (m, 4H), 7.64 (m, 2H), 7.24 (m, 2H), 4.35 (m, 4H), 3.7 (brs, 4H), 3.44 (d, 2H), 2.3 (several s, 12H); MS m/z 1071 (M+1).

Utilizing the foregoing procedure, the following compounds were prepared:

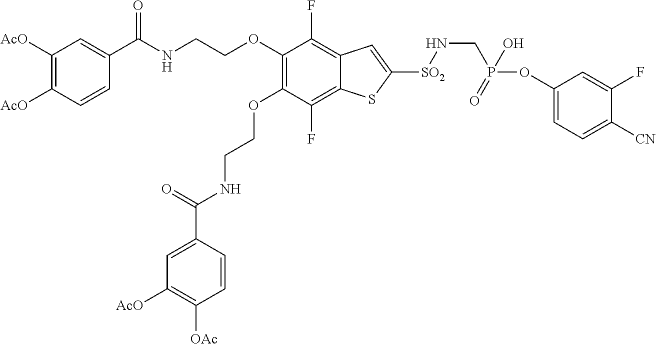

### {2-[({[[4-cyano-3-fluorophenoxy](hydroxy)phosphoryl]methyl}amino)sulfonyl]-4,7-difluoro-1-benzothiene-5,6-diyl}bis(oxyethane-2,1-diyliminocarbonylbenzene-4,1,2-triyl)tetraacetate

¹H NMR (500 MHz, CD₃OD) δ (ppm): 7.88 (d, 1H), 7.76 (m, 2H), 7.7 (dd, 2H), 7.64 (brt, 1H), 7.3 (dd, 2H), 7.16 (m, 2H), 4.4 (two t, 4H), 3.74 (br t, 4H), 3.46 (d, 2H), 2.3 (several s, 12H); MS m/z 1021 (M+1).

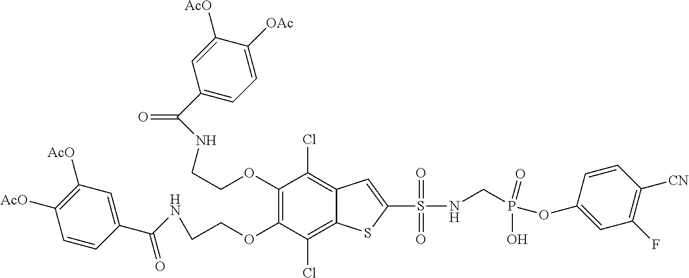

### {2-[({[[4-cyano-3-fluorophenoxy](hydroxy)phosphoryl]methyl}amino)sulfonyl]-4,7-dichloro-1-benzothiene-5,6-diyl}bis(oxyethane-2,1-diyliminocarbonylbenzene-4,1,2-triyl)tetraacetate

¹H NMR (500 MHz, CD₃OD) δ (ppm): 7.90 (s, 1H), 7.75 (m, 2H), 7.70 (m, 2H), 7.60 (m, 1H), 7.30 (m, 2H), 7.15 (m, 2H), 4.40 (t, 2H), 4.32 (t, 2H), 3.78 (m, 4H), 3.50 (d, 2H), 2.35 (d, 12H); MS m/z 1053 (M).

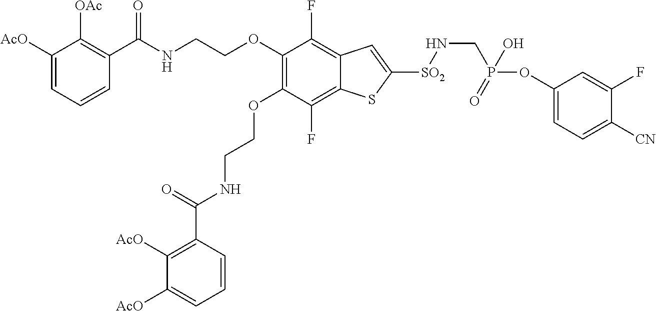

### {2-[({[(4-cyano-3-fluorophenoxy)(hydroxy)phosphoryl]methyl}amino)sulfonyl]-1-benzothiene-5,6-diyl}bis(oxyethane-2,1-diyliminocarbonylbenzene-3,1,2-triyl)tetraacetate

¹H NMR (500 MHz, CD₃OD) δ (ppm): 7.85 (s, 1H), 7.78 (s, 1H), 7.5 (m, 4H), 7.48 (s, 1H), 7.34 (m, 3H), 7.12 (dd, 2H), 4.24 (m, 4H), 3.76 (br t, 4H), 2.28 (s, 6H), 2.18 (s, 6H); MS m/z 985 (M+1).

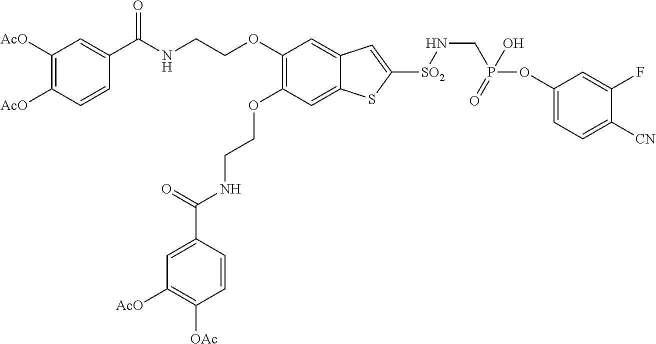

### {2-[({[[4-cyano-3-fluorophenoxy](hydroxy)phosphoryl]methyl}amino)sulfonyl]-1-benzothiene-5,6-diyl}bis(oxyethane-2,1-diyliminocarbonylbenzene-4,1,2-triyl)tetraacetate

¹H NMR (600 MHz, CD₃OD) δ (ppm): 7.76 (d, 2H), 7.7 (m, 3H), 7.5 (br t, 1H), 7.48 (s, 1H), 7.44 (s, 1H), 7.28 (dd, 2H), 7.1 (m, 2H), 4.24 (m, 4H), 3.76 (m, 4H), 3.25 (d, 2H, hidden), 2.26 (several s, 12H); MS m/z 985

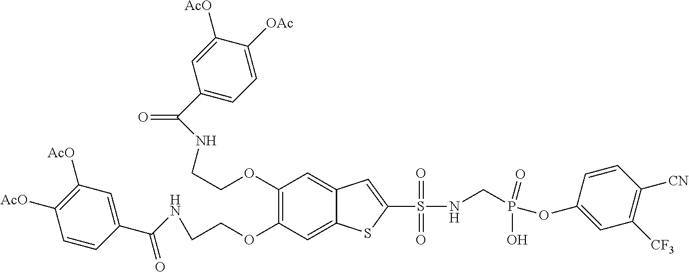

### {2-[({[[4-cyano-3-(trifluoromethyl)phenoxy](hydroxy)phosphoryl]methyl}amino)sulfonyl]-1-benzothiene-5,6-diyl}bis(oxyethane-2,1-diyliminocarbonylbenzene-4,1,2-triyl)tetraacetate

¹H NMR (500 MHz, CD₃OD) δ (ppm): 7.70 (m, 6H), 7.45 (m, 4H), 7.30 (s, 1H), 7.28 (m, 1H), 4.24 (m, 4H), 3.78 (m, 4H), 3.30 (d, 2H), 2.30 (d, 12H); MS m/z 1035 (M+1).

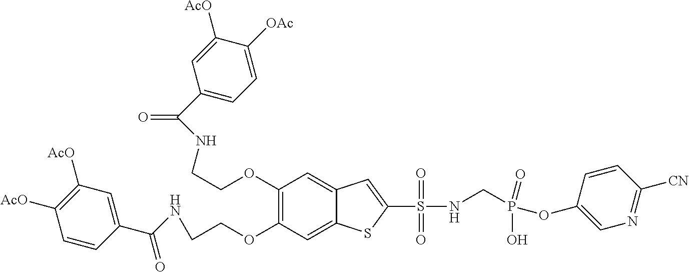

### {2-[({[[(6-cyanopyridin-3-yl)oxy](hydroxy)phosphoryl]methyl}amino)sulfonyl]-1-benzothiene-5,6-diyl}bis(oxyethane-2,1-diyliminocarbonylbenzene-4,1,2-triyl)tetraacetate

¹H NMR (500 MHz, CD₃OD) δ (ppm): 7.78 (m, 4H), 7.64 (m, 1H), 7.50 (m, 3H), 7.30 (m, 3H), 6.90 (m, 1H), 4.26 (m, 4H), 3.80 (m, 4H), 3.30 (d, 2H), 2.30 (d, 12); MS m/z 968 (M+1).

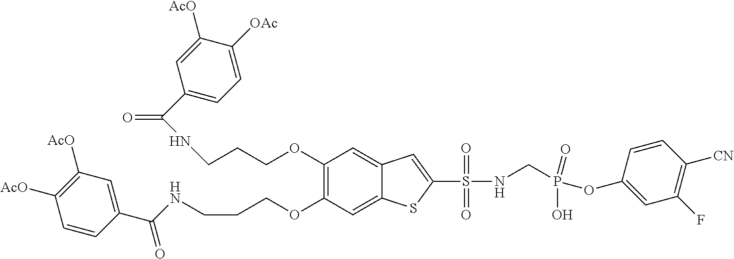

### {2-[({[[4-cyano-3-fluorophenoxy](hydroxy)phosphoryl]methyl}amino)sulfonyl]-1-benzothiene-5,6-diyl}bis(oxypropane-2,1-diyliminocarbonylbenzene-4,1,2-triyl)tetraacetate

¹H NMR (500 MHz, CD₃OD) δ (ppm): 7.72 (m, 4H), 7.66 (s, 1H), 7.50 (m, 1H), 7.42 (s, 1H), 7.30 (m, 1H), 7.20 (d, 2H), 7.12 (m, 2H), 4.18 (m, 4H), 3.62 (m, 4H), 3.40 (d, 2H), 3.30 (d, 12H), 2.14 (m, 4H); MS m/z 1013 (M+1).

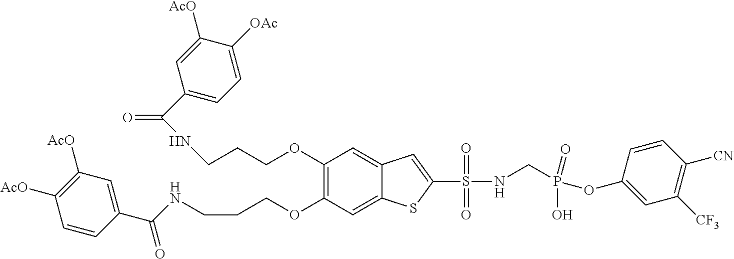

### {2-[({[[4-cyano-3-(trifluoromethyl)phenoxy](hydroxy)phosphoryl]methyl}amino)sulfonyl]-1-benzothiene-5,6-diyl}bis(oxypropane-2,1-diyliminocarbonylbenzene-4,1,2-triyl)tetraacetate

¹H NMR (500 MHz, CD₃OD) δ (ppm): 7.70 (m, 6H), 7.50 (m, 1H), 7.40 (m, 3H), 7.30 (m, 2H), 4.18 (m, 4H), 3.65 (m, 4H), 3.30 (d, 2H), 2.30 (d, 12H), 2.10 (m, 4H); MS m/z 1063 (M+1).

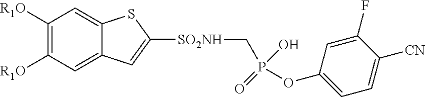

R₁ and R₂ independently represent:

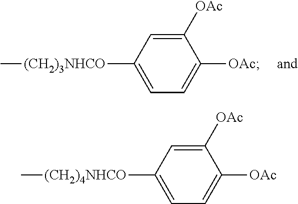

### 2-(acetyloxy)-4-({[3-({6-(4-{[3,4-bis(acetyloxy)benzoyl]amino}butoxy)-2-[({[(4-cyano-3-fluorophenoxy)-(hydroxy)phosphoryl]methyl}amino)sulfonyl]-1-benzothien-5-yl}oxy)propyl]amino}carbonyl)phenyl acetate and 2-(acetyloxy)-4-({[3-({6-(4-{[3,4-bis(acetyloxy)benzoyl]amino}propoxy)-2-[({[(4-cyano-3-fluorophenoxy)-(hydroxy)-phosphoryl]methyl}amino)sulfonyl]-1-benzothien-5-yl}oxy)butyl]amino}carbonyl)phenyl acetate

Major isomer (0.0031 g): ¹H NMR (500 MHz, CD₃OD) δ (ppm): 7.69 (dd, 1H), 7.68 (s, 1H), 7.61 (d, 1H), 7.44 (t, 1H), 7.38 (s, 1H), 7.34 (s, 1H), 7.27 (d, 1H), 7.19 (d, 1H), 7.09 (dd, 1H), 7.03 (dd, 1H), 4.15 (t, 2H), 4.08 (t, 2H), 3.60 (t, 2H), 3.46 (t, 2H), 3.26 (d, 2H), 2.26 (d, 6H), 2.24 (s, 3H), 2.22 (s, 3H), 2.11 (m, 2H), 1.84 (m, 4H); MS m/z 1028 (M+1);

Minor isomer (0.0022 g): ¹H NMR (500 MHz, CD₃OD) δ (ppm): 7.69 (dd, 1H), 7.67 (s, 1H), 7.60-7.65 (m, 3H), 7.49 (t, 1H), 7.38 (s, 1H), 7.33 (s, 1H), 7.27 (d, 1H), 7.17 (d, 1H), 7.11 (dd, 1H), 7.06 (dd, 1H), 4.12 (m, 4H), 3.61 (t, 2H), 3.45 (t, 2H), 3.34 (d, 2H), 2.26 (d, 6H), 2.24 (s, 3H), 2.22 (s, 3H), 2.10 (m, 2H), 1.84 (m, 4H); MS m/z 1028 (M+1).

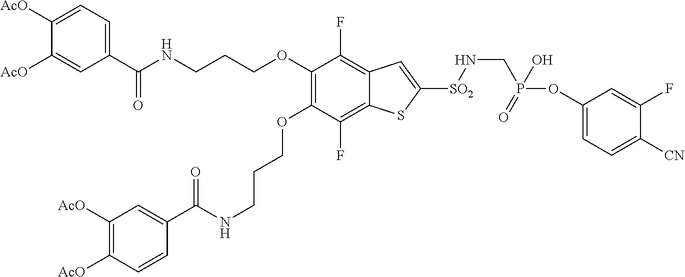

### {2-[({[[4-cyano-3-fluorophenoxy](hydroxy)phosphoryl]methyl}amino)sulfonyl]-4,7-d fluoro-1-benzothiene-5,6-diyl}bis(oxypropane-2,1-diyliminocarbonylbenzene-4,1,2-triyl)tetraacetate

¹H NMR (600 MHz, CD₃OD) δ (ppm): 7.86 (brs 1H), 7.72 (dd, 2H), 7.68 (brs, 2H), 7.6 (br t, 1H), 7.28 (d, 2H), 7.12 (m, 2H), 4.3 (two t, 4H), 3.6 (t, 4H), 3.5 (brs, 2H), 2.25 (several s, 12H), 2.1 (m, 4H); MS m/z 1049 (M+1).

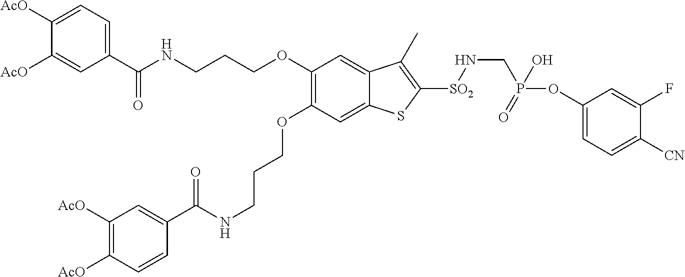

### {2-[({[[4-cyano-3-fluorophenoxy](hydroxy)phosphoryl]methyl}amino)sulfonyl]-3-methyl-1-benzothiene-5,6-diyl}bis(oxypropane-2,1-diyliminocarbonylbenzene-4,1,2-triyl)tetraacetate

¹H NMR (500 MHz, CD₃OD) δ (ppm): 7.73 (d, 2H), 7.68 (s, 2H), 7.46 (t, 1H), 7.38 (s, 1H), 7.3 (d, 2H), 7.24 (s, 1H), 7.04 (m, 2H), 4.19 (m, 4H), 3.64 (m, 4H), 3.44 (d, 2H), 2.58 (s, 3H), 2.29 (two s, 12H), 2.1 (m, 4H); MS m/z 1027 (M+1).

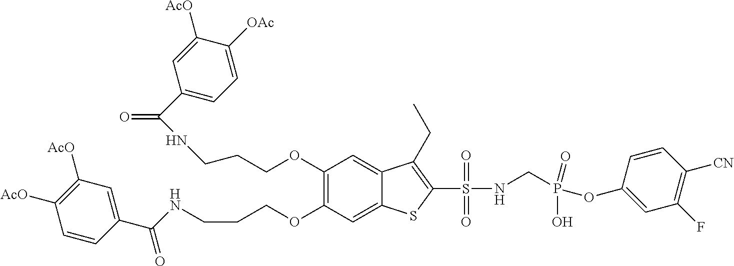

### {2-[({[[4-cyano-3-fluorophenoxy](hydroxy)phosphoryl]methyl}amino)sulfonyl]-3-ethyl-1-benzothiene-5,6-diyl}bis(oxypropane-2,1-diyliminocarbonylbenzene-4,1,2-triyl)tetraacetate

¹H NMR (500 MHz, CD₃OD) δ (ppm): 7.72 (m, 2H), 7.68 (m, 2H), 7.46 (m, 1H), 7.40 (s, 1H), 7.30 (d, 2H), 7.26 (s, 1H), 7.06 (m, 2H), 4.20 (m, 4H), 3.62 (m, 4H), 3.20 (d, 2H), 3.10 (t, 3H), 2.30 (d, 12H), 2.10 (m, 4H), 1.26 (t, 3H); MS m/z 1041 (M+1).

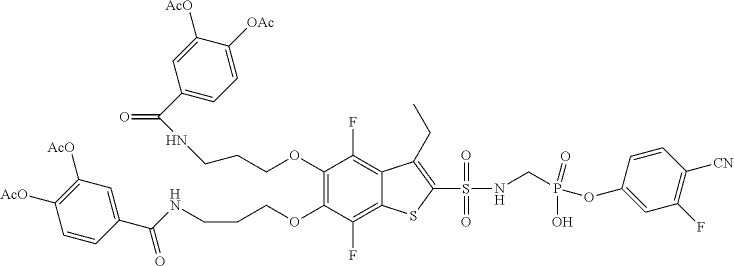

### {2-[({[[4-cyano-3-fluorophenoxy](hydroxy)phosphoryl]methyl}amino)sulfonyl]-4,7-difluoro-3-ethyl-1-benzothiene-5,6-diyl}bis(oxypropane-2,1-diyliminocarbonylbenzene-4,1,2-triyl)tetraacetate

¹H NMR (500 MHz, CD₃OD) δ (ppm): 7.78 (m, 2H), 7.70 (d, 2H), 7.60 (m, 1H), 7.30 (d, 2H), 7.10 (m, 2H), 4.40 (t, 2H), 4.30 (t, 2H), 3.66 (m, 4H), 3.50 (d, 2H), 3.20 (q, 2H), 2.30 (d, 12H), 2.18 (m, 4H), 1.30 (t, 3H); MS m/z 1077 (M+1).

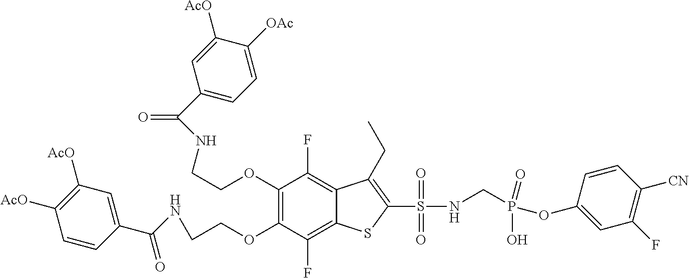

### {2-[({[[4-cyano-3-fluorophenoxy](hydroxy)phosphoryl]methyl}amino)sulfonyl]-4,7-difluoro-3-ethyl-1-benzothiene-5,6-diyl}bis(oxyethane-2,1-diyliminocarbonylbenzene-4,1,2-triyl)tetraacetate

¹H NMR (500 MHz, CD₃OD) δ (ppm): 7.70 (m, 2H), 7.64 (d, 2H), 7.58 (m, 1H), 7.28 (m, 2H), 7.10 (m, 2H), 4.40 (t, 2H), 4.30 (t, 2H), 3.70 (m, 4H), 3.40 (d, 2H), 3.10 (q, 2H), 2.26 (d, 12H), 1.20 (t, 3H); MS m/z 1049 (M+1).

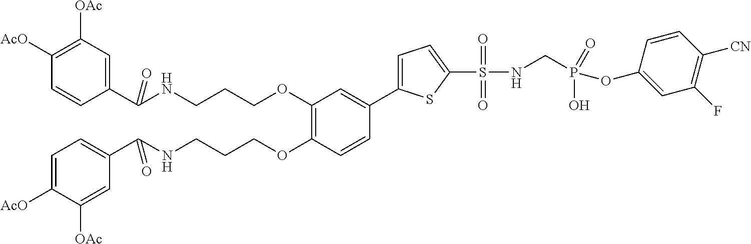

### (4-{5-[({[(4-cyano-3-fluorophenoxy)(hydroxy)phosphoryl]methyl}amino)sulfonyl]-2-thienyl}-1,2-phenylene)bis(oxypropane-3,1-diyliminocarbonylbenzene-4,1,2-triyl)tetraacetate

¹H NMR (500 MHz, CD₃OD) δ (ppm): 7.72 (m, 2H), 7.64 (m, 3H), 7.54 (s, 1H), 7.30 (m, 3H), 7.20 (m, 4H), 7.00 (d, 1H), 4.18 (q, 4H), 3.60 (q, 4H), 3.40 (d, 2H), 2.30 (m, 12H), 2.08 (m, 4H); MS m/z 1039 (M+1).

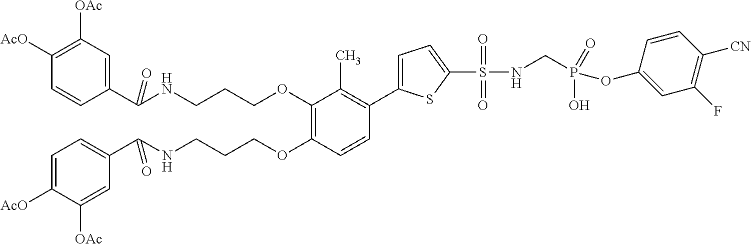

### (4-{5-[({[(4-cyano-3-fluorophenoxy)(hydroxy)phosphoryl]methyl}amino)sulfonyl]-2-thienyl}-3-methyl-1,2-phenylene)bis(oxypropane-3,1-diyliminocarbonylbenzene-4,1,2-triyl)tetraacetate

¹H NMR (500 MHz, CD₃OD) δ (ppm): 7.74 (m, 2H), 7.64 (m, 2H), 7.62 (m, 1H), 7.60 (d, 1H), 7.30 (m, 2H), 7.22 (m, 1H), 7.20 (m, 1H), 7.10 (d, 1H), 7.05 (d, 1H), 6.96 (d, 1H), 4.10 (t, 2H), 4.08 (t, 2H), 3.66 (t, 2H), 3.60 (t, 2H), 3.26 (d, 2H), 2.30 (m, 12H), 2.10 (m, 4H); MS m/z 1053 (M+1).

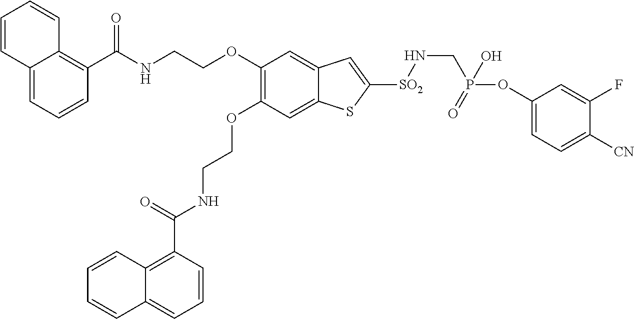

### 4-cyano-3-fluorophenyl hydrogen {[({5,6-bis[2-(1-naphthoylamino)ethoxy]-1-benzothien-2-yl}sulfonyl)amino]-methyl}phosphonate

¹H NMR (600 MHz, CD₃OD) δ (ppm): 8.54 (m, 1H), 8.1 (d, 2H), 7.8 (dd, 2H), 7.74-7.3 (m, 13H), 7.05 (dd, 2H), 4.28 (m, 4H), 3.78 (m, 4H), 3.26 (brs, 2H); MS m/z 853 (M+1).

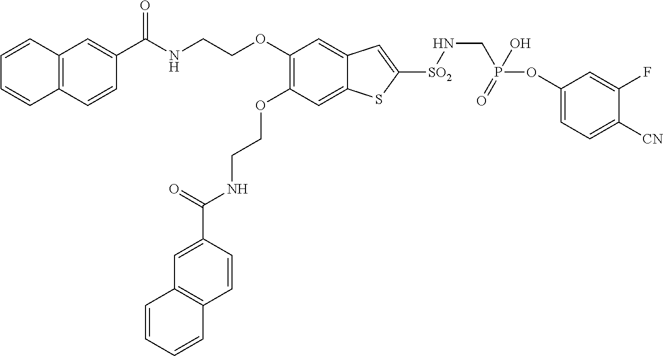

### 4-cyano-3-fluorophenyl hydrogen {[({5,6-bis[2-(2-naphthoylamino)ethoxy]-1-benzothien-2-yl}sulfonyl)amino]-methyl}phosphonate

¹H NMR (500 MHz, CD₃OD) δ (ppm): 8.36 (s, 1H), 7.89-7.4 (m, 17H), 7.1 (dd, 2H), 4.34 (m, 4H), 3.90 (m, 4H), 3.36 (d, 2H, hidden); MS m/z 853 (M+1).

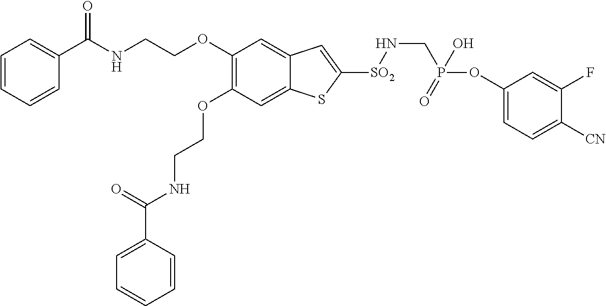

### 4-cyano-3-fluorophenyl hydrogen {[({5,6-bis[2-(benzoylamino)ethoxy]-1-benzothien-2-yl}sulfonyl)amino]-methyl}phosphonate

¹H NMR (600 MHz, CD₃OD) δ (ppm): 7.8 (d, 4H), 7.7 (s, 1H), 7.5-7.3 (m, 9H), 7.1 (m, 2H), 4.25 (m, 4H), 3.8 (m, 4H), 3.35 (brs, 2H); MS m/z 753 (M+1).

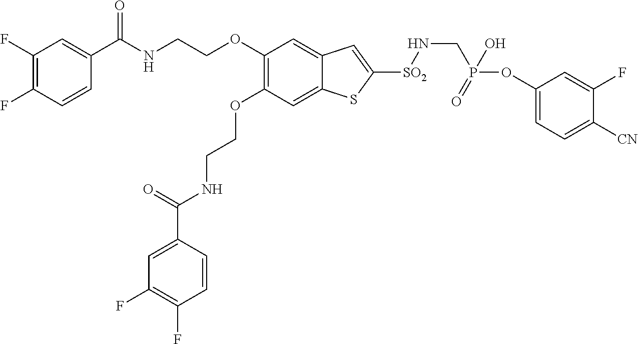

### 4-cyano-3-fluorophenyl hydrogen {[({5,6-bis[2-(3,4-difluorobenzoylamino)ethoxy]-1-benzothien-2-yl}-sulfonyl)amino]methyl}phosphonate

¹H NMR (500 MHz, CD₃OD) δ (ppm): 7.8-7.3 (m, 11H), 7.05 (dd, 2H), 4.3 (m, 4H), 3.8 (m, 4H), 3.3 (d, 2H); MS m/z 825 (M+1).

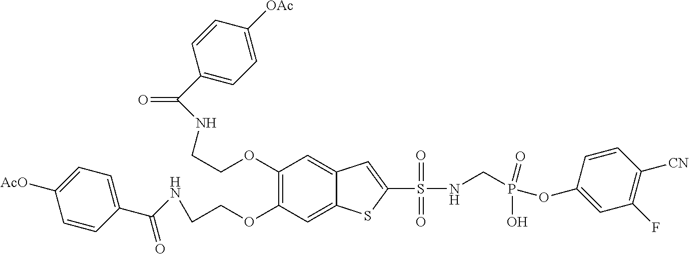

### {2-[({[(4-cyano-3-fluorophenoxy)(hydroxy)phosphoryl]methyl}amino)sulfonyl]-1-benzothiene-5,6-diyl}bis(oxyethane-2,1-diyliminocarbonyl-4,1-phenylene) diacetate

¹H NMR (500 MHz, CD₃OD) δ (ppm): 7.90 (d, 4H), 7.74 (s, 1H), 7.58 (m, 2H), 7.46 (s, 1H), 7.20 (m, 4H), 7.12 (m, 2H), 4.30 (m, 4H), 3.80 (m, 4H), 3.40 (d, 2H), 2.30 (s, 6H); MS m/z 869 (M+1).

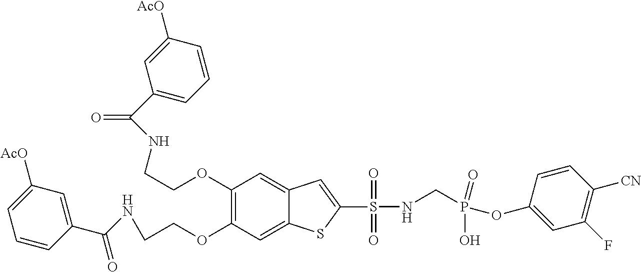

### {2-[({[(4-cyano-3-fluorophenoxy)(hydroxy)phosphoryl]methyl}amino)sulfonyl]-1-benzothiene-5,6-diyl}bis(oxyethane-2,1-diyliminocarbonyl-3,1-phenylene) diacetate

¹H NMR (500 MHz, CD₃OD) δ (ppm): 7.76 (m, 3H), 7.60 (s, 2H), 7.50 (m, 5H), 7.30 (m, 2H), 7.16 (m, 2H), 4.30 (m, 4H), 3.80 (m, 4H), 3.36 (d, 2H), 2.30 (s, 6H); MS m/z 869 (M+1).

Substituting the phenylacetic acid for benzoic acid, the following compound was prepared:

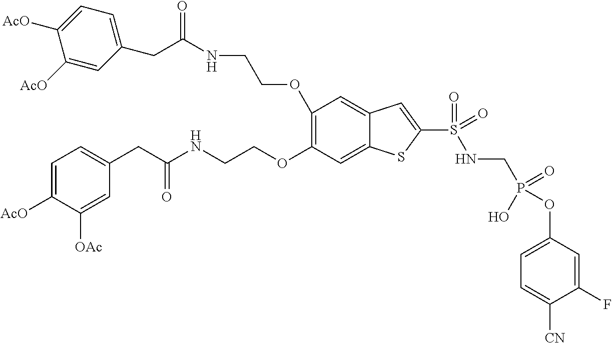

### {2-[({[(4-cyano-3-fluorophenoxy)(hydroxy)phosphoryl]methyl}amino)sulfonyl]-1-benzothiene-5,6-diyl}bis[oxyethane-2,1-diylimino(2-oxoethane-2,1-diyl)benzene-4,1,2-triyl]tetraacetate

¹H NMR (500 MHz, CD₃OD) δ (ppm): 7.8 (s, 1H), 7.5 (t, 1H), 7.4 (s, 1H), 7.35 (s, 1H), 7.2-7.05 (m, 8H), 4.15 (m, 4H), 3.6 (m, 8H), 2.3 (s, 6H), 2.1 (s, 6H); MS m/z 1013 (M+1).

### Example 2

### Preparation of 4-(cyano-3-fluorophenyl hydrogen {5,6-bis[3-(3,4-dihydroxybenzamido)propoxy]benzo[d]thiazole-2-sulfonamido}-methylphosphonate

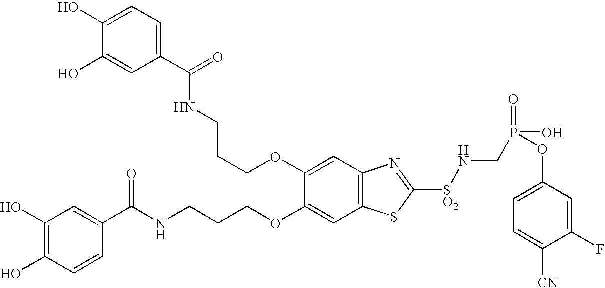

Step 1. A mixture of 3,4-bis(methoxymethoxy)benzoic acid (4 mg, 0.029 mmol), diisopropylethylamine (0.005 mL, 0.029 mmol) and HATU (11.4 mg, 0.030 mmol) in DMF (1 mL) was stirred at 0° C.-5° C. under N₂. To this mixture was added a suspension of compound 4-cyano-3-fluorophenyl hydrogen (5,6-bis(2-ammoniopropoxy)benzo[d]thiazole-2-sulfonamido)methylphosphonate trifluoroacetate (6.6 mg, 0.012 mmol) in diisopropylethylamine (0.006 mL, 0.03 mmol) and DMF (5 mL). The reaction mixture was stirred at 0° C.-5° C. for 10 min, then at ambient temperature for 2 h. Purification by preparative HPLC (Thermo, Aquasil C18, 240×21.2 mm, 5 μm; gradient MeOH/H₂O 10/90 to 95/5 both containing 0.05% HCO₂H, over 30 min) gave 12 mg of intermediate, as a pale yellow solid.

MS m/z 1020 (M−1).

Step 2. The solid was then dissolved in methanol (2 mL) and a 3M solution of HCl in MeOH (0.02 mL 6 mmol) was added under N₂. The reaction mixture was stirred at ambient temperature for 2 h and purified by preparative HPLC (Thermo, Aquasil C18, 240×21.2 mm, 5 μM; gradient 10/90 to 95/5 MeOH/H₂O both containing 0.05% HCO₂H, over 30 min), three times, to give 1 mg of product as a white film.

MS m/z 844 (M−1).

### Example 3

### Preparation of 4-cyano-3-(trifluoromethyl)phenyl hydrogen ({[(5,6-bis{2-[(3,4-dihydroxybenzoyl)amino]ethoxy}-4,7-difluoro-1-benzothien-2-yl)-sulfonyl]amino}methyl)phosphonate

A methanolic solution of {2-[({[[4-cyano-3-(trifluoromethyl)phenoxy](hydroxy)phosphoryl]methyl}-amino)-sulfonyl]-4,7-difluoro-1-benzothiene-5,6-diyl}bis(oxyethane-2,1-diyliminocarbonylbenzene-4,1,2-triyl)tetraacetate (40 mg, 0.037 mmol) was purged with N₂ for 15 min at 0° C., and then NaOMe (5 equiv.) was added. The reaction mixture was stirred further for 10 minutes and then neutralized with HOAc. Purification by reverse phase HPLC afforded the product (16 mg, 50%).

¹H NMR (600 MHz, CD₃OD) δ (ppm): 7.9 (d, 1H), 7.85 (s, 1H), 7.7 (s, 1H), 7.55 (d, 1H), 7.3 (d, 2H), 7.2 (brt, 2H), 6.8 (m, 2H), 4.35 (m, 4H), 3.65 (brs, 4H), 3.45 (brs, 2H); MS m/z 903 (M+1).

Utilizing the foregoing procedure, the following compounds were prepared:

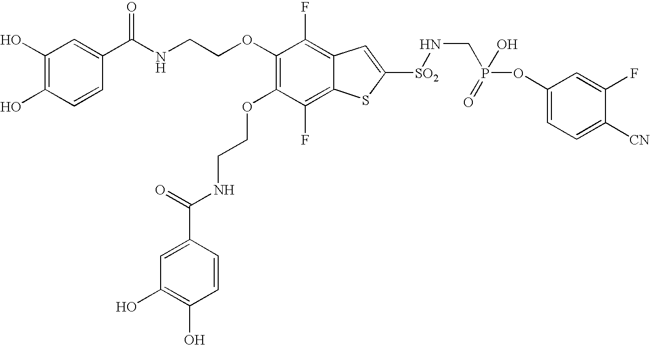

### 4-cyano-3-fluorophenyl hydrogen ({[(5,6-bis {2-[(3,4-dihydroxybenzoyl)amino]ethoxy}-4,7-difluoro-1-benzothien-2-yl)sulfonyl]amino}methyl)phosphonate

¹H NMR (500 MHz, CD₃OD) δ (ppm): 7.84 (brs, 1H), 7.52 (br s, 1H), 7.3 (brs, 2H), 7.2 (m, 4H), 6.8 (m, 2H), 4.4 (two t, 4H), 3.7 (brs, 4H), 3.34 (brs, 2H, hidden); MS m/z 853 (M+1).

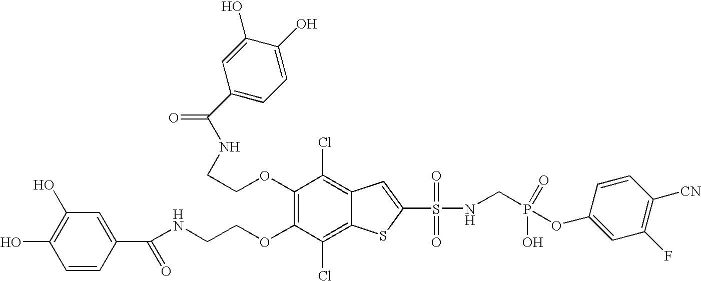

### 4-cyano-3-fluorophenyl hydrogen ({[(5,6-bis {2-[3,4-dihydroxybenzoyl)amino]ethoxy}-4,7-dichloro-1-benzothien-2-yl)sulfonyl]amino}methyl)phosphonate

¹H NMR (500 MHz, CD₃OD) δ (ppm): 7.90 (s, 1H), 7.60 (m, 1H), 7.32 (m, 2H), 7.22 (m, 2H), 7.12 (m, 2H), 6.80 (m, 2H), 4.36 (t, 2H), 4.30 (t, 2H), 3.75 (m, 4H), 3.40 (d, 2H); MS m/z 885 (M+1).

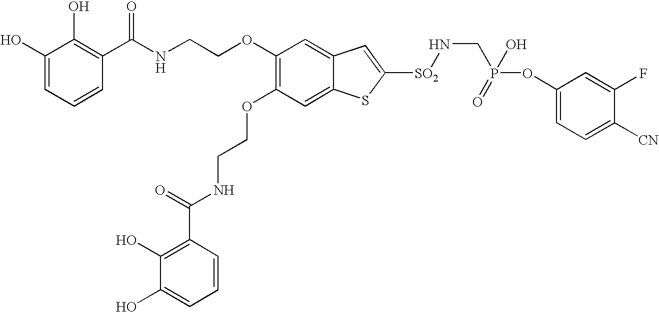

### 4-cyano-3-fluorophenyl hydrogen ({[(5,6-bis{2-[(2,3-dihydroxybenzoyl)amino]ethoxy}-1-benzothien-2-yl)-sulfonyl]amino}methyl)phosphonate

¹H NMR (600 MHz, CD₃OD) δ (ppm): 7.68 (s, 1H), 7.5 (s, 1H), 7.45 (s, 1H), 7.42 (m, 1H), 7.2 (d, 2H), 7.1 (d, 1H), 7.04 (d, 1H), 6.9 (d, 2H), 6.68 (m, 2H), 4.28 (m, 4H), 3.82 (m, 4H), 3.22 (d, 2H); MS m/z 817 (M+1).

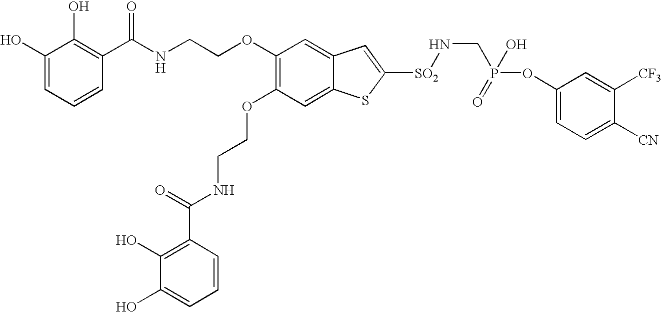

### 4-cyano-3-(trifluoromethyl)phenyl hydrogen ({[(5,6-bis{2-[(2,3-dihydroxybenzoyl)amino]ethoxy}-1-benzothien-2-yl)-sulfonyl]amino}methyl)phosphonate

¹H NMR (600 MHz, CD₃OD) δ (ppm): 7.7 (m, 2H), 7.68 (s, 1H), 7.48 (m, 2H), 7.44 (s, 1H), 7.2 (d, 2H), 6.9 (d, 2H), 6.68 (br t, 2H), 4.26 (m, 4H), 3.8 (m, 4H), 3.24 (d, 2H); MS m/z 867 (M+1).

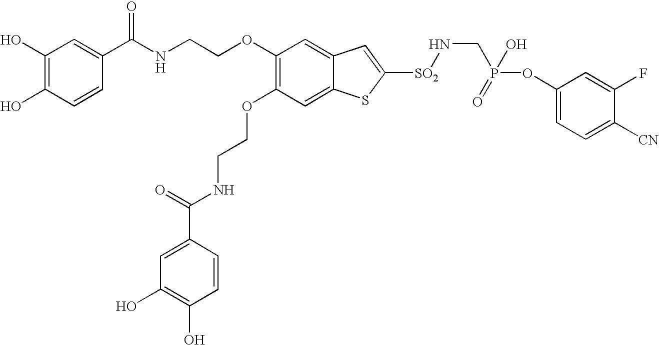

### 4-cyano-3-fluorophenyl hydrogen ({[(5,6-bis{2-[(3,4-dihydroxybenzoyl)amino]ethoxy}-1-benzothien-2-yl)-sulfonyl]amino}methyl)phosphonate

¹H NMR (600 MHz, CD₃OD) δ (ppm): 7.54 (s, 1H), 7.08 (d, 2H), 7.3 (brs, 2H), 7.2 (d, 2H), 7.1 (m, 2H), 6.8 (d, 2H), 4.2 (m, 4H), 3.15 (m, 4H), 3.3 (d, 2H, hidden); MS m/z 817 (M+1).

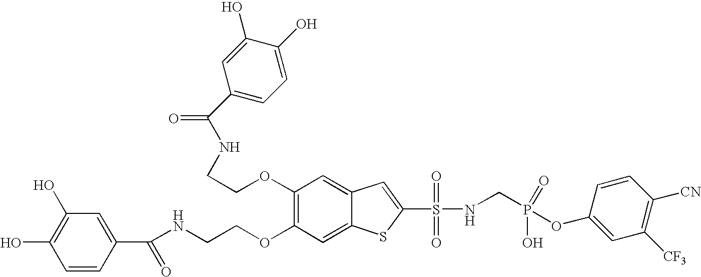

### 4-cyano-3-(trifluoromethyl)phenyl hydrogen ({[(5,6-bis{2-[(3,4-dihydroxybenzoyl)amino]ethoxy}-1-benzothien-2-yl)sulfonyl]amino}methyl)phosphonate

¹H NMR (500 MHz, CD₃OD) δ (ppm): 7.70 (m, 3H), 7.45 (m, 3H), 7.30 (s, 2H), 7.20 (d, 2H), 6.80 (d, 2H), 4.24 (m, 4H), 3.78 (m, 4H), 3.30 (d, 2H); MS m/z 867 (M+1).

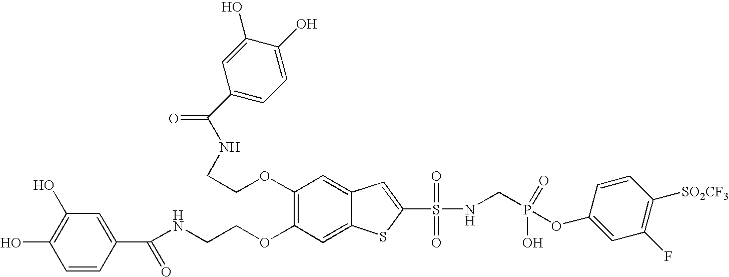

### 3-fluoro-4-[(trifluoromethyl)sulfonyl]phenyl hydrogen ({[(5,6-bis{2-[(3,4-dihydroxybenzoyl)amino]ethoxy}-1-benzothien-2-yl)sulfonyl]amino}methyl)phosphonate

¹H NMR (500 MHz, CD₃OD) δ (ppm): 7.70 (m, 2H), 7.64 (d, 2H), 7.30 (m, 3H), 7.20 (m, 2H), 6.78 (m, 2H), 4.20 (m, 4H), 3.78 (m, 4H), 3.30 (d, 2H); MS m/z 924 (M+1).

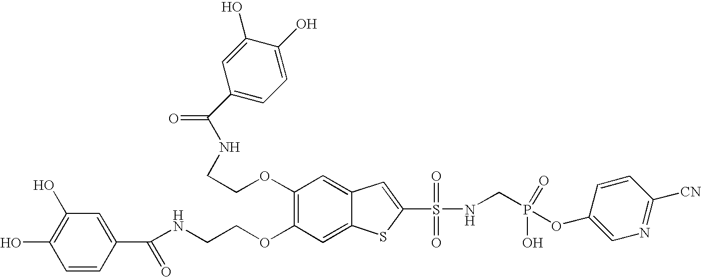

### 6-cyanopyridin-3-yl hydrogen ({[(5,6-bis{2-[3,4-dihydroxybenzoyl)amino]ethoxy}-1-benzothien-2-yl)sulfonyl]amino}methyl)phosphonate

¹H NMR (500 MHz, CD₃OD) δ (ppm): 7.68 (m, 2H), 7.40 (m, 3H), 7.30 (m, 2H), 7.20 (m, 2H), 6.80 (m, 3H), 4.20 (m, 4H), 3.76 (m, 4H), 3.30 (d, 2H); MS m/z 800 (M+1).

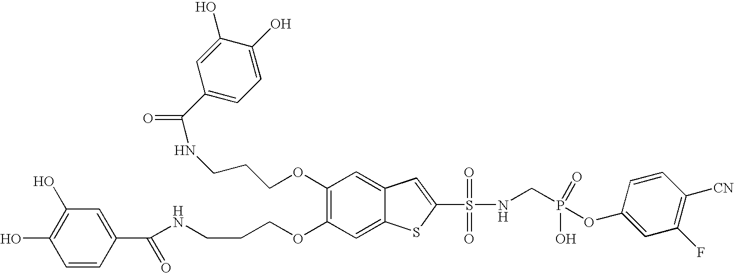

### 4-cyano-3-fluorophenyl hydrogen ({[(5,6-bis{2-[(3,4-dihydroxybenzoyl)amino]propoxy}-1-benzothien-2-yl)-sulfonyl]amino}methyl)phosphonate

¹H NMR (500 MHz, CD₃OD) δ (ppm): 7.80 (s, 1H), 7.52 (m, 1H), 7.40 (d, 2H), 7.30 (s, 2H), 7.20 (d, 2H), 7.10 (m, 2H), 6.80 (d, 2H), 4.18 (m, 4H), 3.60 (m, 4H), 3.40 (d, 2H), 2.10 (m, 4H); MS m/z 845 (M+1).

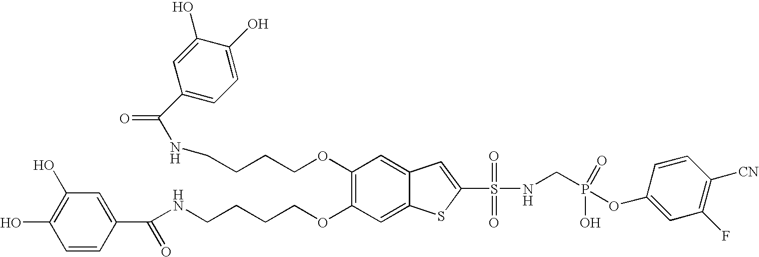

### 4-cyano-3-fluorophenyl hydrogen ({[(5,6-bis{2-[(3,4-dihydroxybenzoyl)amino]butoxy}-1-benzothien-2-yl)-sulfonyl]amino}methyl)phosphonate

¹H NMR (500 MHz, CD₃OD) δ (ppm): 7.70 (s, 1H), 7.42 (m, 1H), 7.40 (d, 2H), 7.30 (s, 2H), 7.20 (d, 2H), 7.10 (m, 2H), 6.80 (d, 2H), 4.18 (m, 4H), 3.42 (m, 4H), 3.30 (d, 2H), 1.94 (m, 4H), 1.82 (m, 4H); MS m/z 873 (M+1).

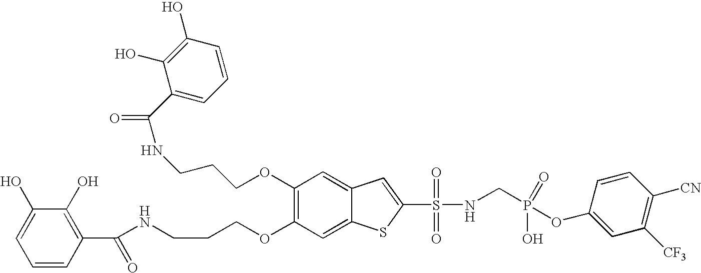

### 4-cyano-3-(trifluoromethyl)phenyl hydrogen ({[(5,6-bis{2-[(2,3-dihydroxybenzoyl)amino]propoxy}-1-benzothien-2-yl)sulfonyl]amino}methyl)phosphonate

¹H NMR (500 MHz, CD₃OD) δ (ppm): 7.70 (m, 3H), 7.45 (m, 3H), 7.20 (d, 2H), 6.92 (d, 2H), 6.70 (t, 2H), 4.20 (m, 4H), 3.65 (m, 4H), 3.30 (d, 2H), 2.15 (m, 4H); MS m/z 895 (M+1).

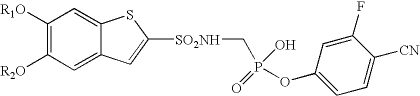

R₁ and R₂ independently represent:

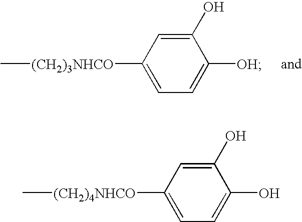

### 4-cyano-3-fluorophenyl hydrogen ({[(6-{4-[(3,4-dihydroxybenzoyl)amino]butoxy}-5-{3-[(3,4-dihydroxybenzoyl)amino]propoxy}-1-benzothien-2-yl)sulfonyl]amino}methyl)phosphonate and 4-cyano-3-fluorophenyl hydrogen ({[(5-{-4-[(3,4-dihydroxybenzoyl)amino]butoxy}-6-{3-[(3,4-dihydroxybenzoyl)amino]propoxy}-1-benzothien-2-yl]sulfonyl}amino)methyl)phosphonate

Purification by HPLC (X-terra Prep MS C18 column; 30×100 mm, 5 micron, flow rate=30 mL/min, 10% to 70% CH₃CN+0.05% TFA/water+0.05% TFA over 15 min. then 70% to 100% CH₃CN+0.05% TFA/water+0.05% TFA over 5 min.)

Major isomer (63% yield): ¹H NMR (500 MHz, CD₃OD) δ (ppm): 7.66 (s, 1H), 7.42 (t, 1H), 7.40 (s, 1H), 7.34 (s, 1H), 7.25 (m, 2H), 7.16 (m, 2H), 7.09 (dd, 1H), 7.02 (dd, 1H), 6.74 (m, 2H), 4.17 (t, 2H), 4.08 (t, 2H), 3.57 (t, 2H), 3.42 (t, 2H), 3.22 (d, 2H), 2.11 (m, 2H), 1.82 (m, 4H); MS m/z 859 (M⁺).

Minor isomer (60% yield): ¹H NMR (500 MHz, CD₃OD) δ (ppm): 7.68 (s, 1H), 7.50 (t, 1H), 7.38 (s, 1H), 7.36 (s, 1H), 7.25 (m, 2H), 7.16 (m, 2H), 7.11 (dd, 1H), 7.06 (dd, 1H), 6.74 (m, 2H), 4.13 (t, 2H), 4.10 (t, 2H), 3.57 (t, 2H), 3.42 (t, 2H), 3.15 (d, 2H), 2.10 (m, 2H), 1.92 (m, 4H); MS m/z 859 (M⁺).

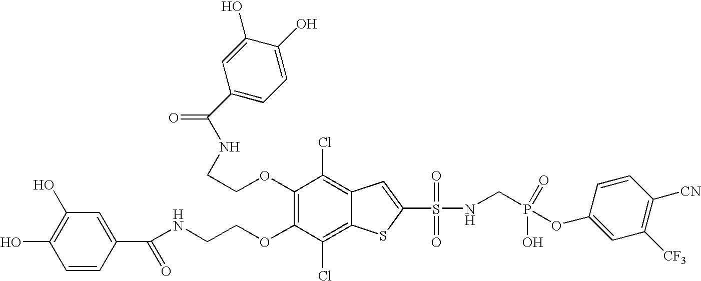

### 4-cyano-3-(trifluoromethyl)phenyl hydrogen ({[(5,6-bis{2-[(3,4-dihydroxybenzoyl)amino]propoxy}-4,7-dichloro-1-benzothien-2-yl)sulfonyl]amino}methyl)phosphonate

¹H NMR (500 MHz, CD₃OD) δ (ppm): 7.90 (s, 1H), 7.80 (m, 1H), 7.72 (m, 1H), 7.52 (m, 1H), 7.30 (m, 2H), 7.20 (m, 2H), 6.80 (m, 2H), 4.36 (t, 2H), 4.30 (t, 2H), 3.70 (m, 4H), 3.38 (d, 2H); MS m/z 935 (M).

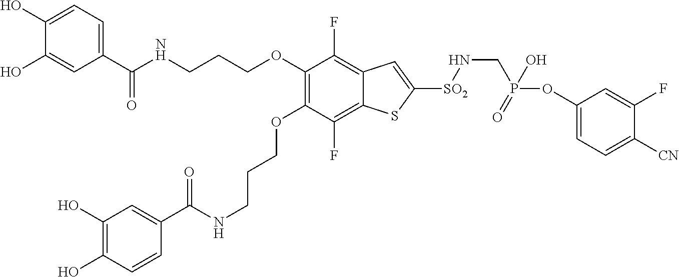

### 4-cyano-3-fluorophenyl hydrogen ({[(5,6-bis{2-[(3,4-dihydroxybenzoyl)amino]propoxy}-4,7-difluoro-1-benzothien-2-yl)sulfonyl]amino}methyl)phosphonate

¹H NMR (500 MHz, CD₃OD) δ (ppm): 7.9 (s, 1H), 7.65 (br t, 1H), 7.3 (s, 2H), 7.2 (d, 2H), 7.15 (m, 2H), 6.8 (d, 2H), 4.3 (m, 4H), 3.6 (m, 4H), 3.5 (brs, 2H), 2.1 (m, 4H); MS m/z 881 (M+1).

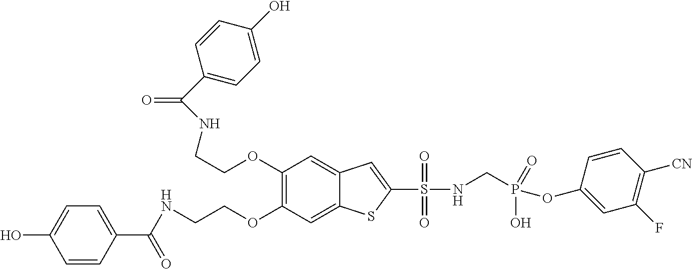

### 4-cyano-3-fluorophenyl hydrogen ({[(5,6-bis{2-[(4-hydroxybenzoyl)amino]ethoxy}-1-benzothien-2-yl)-sulfonyl]amino}methyl)phosphonate

¹H NMR (500 MHz, CD₃OD) δ (ppm): 7.78 (m, 4H), 7.60 (t, 1H), 7.46 (d, 2H), 7.14 (m, 3H), 6.80 (m, 4H), 4.24 (m, 4H), 3.80 (m, 4H), 3.40 (d, 2H); MS m/z 785 (M+1).

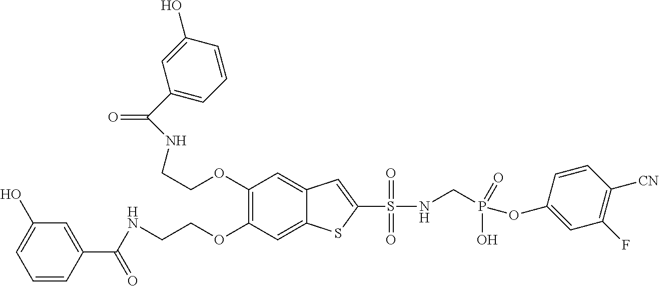

### 4-cyano-3-fluorophenyl hydrogen ({[(5,6-bis{2-[(3-hydroxybenzoyl)amino]ethoxy}-1-benzothien-2-yl)-sulfonyl]amino}methyl)phosphonate

¹H NMR (500 MHz, CD₃OD) δ (ppm): 7.78 (s, 1H), 7.58 (t, 1H), 7.50 (d, 2H), 7.26 (m, 6H), 7.14 (m, 2H), 6.98 (d, 2H), 4.24 (m, 4H), 3.80 (m, 4H), 3.38 (d, 2H); MS m/z 785 (M+1).

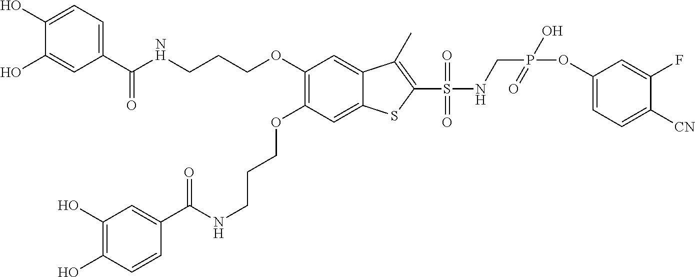

### 4-cyano-3-fluorophenyl hydrogen ({[(5,6-bis{2-[(3,4-dihydroxybenzoyl)amino]propoxy}-3-methyl-1-benzothien-2-yl)sulfonyl]amino}methyl)phosphonate

¹H NMR (500 MHz, CD₃OD) δ (ppm): 7.5 (t, 1H), 7.4 (s, 1H), 7.3 (dd, 2H), 7.24 (s, 1H), 7.22 (m, 2H), 7.05 (m, 2H), 6.8 (d, 2H), 4.2 (m, 4H), 3.6 (m, 4H), 3.5 (d, 2H), 2.59 (s, 3H), 2.12 (m, 4H); MS m/z 859 (M+1).

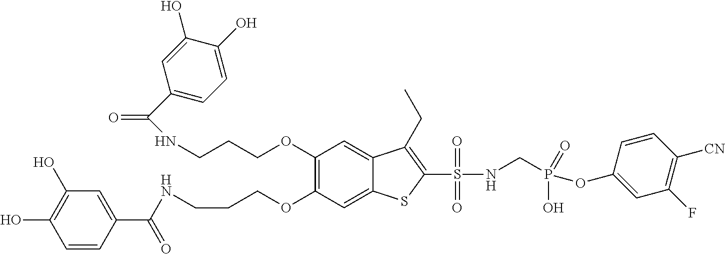

### 4-cyano-3-fluorophenyl hydrogen ({[(5,6-bis{2-[(3,4-dihydroxybenzoyl)amino]propoxy}-3-ethyl-1-benzothien-2-yl)sulfonyl]amino}methyl)phosphonate

¹H NMR (500 MHz, CD₃OD) δ (ppm): 7.50 (t, 1H), 7.40 (s, 1H), 7.30 (m, 2H), 7.22 (s, 1H), 7.20 (m, 2H), 7.05 (m, 2H), 6.80 (d, 2H), 4.20 (m, 4H), 3.60 (m, 4H), 3.40 (d, 2H), 3.10 (q, 2H), 2.10 (m, 4H), 1.25 (t, 3H); MS m/z 873 (M+1).

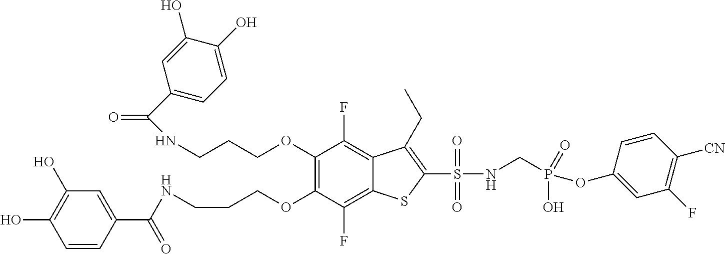

### 4-cyano-3-fluorophenyl hydrogen ({[(5,6-bis{2-[(3,4-dihydroxybenzoyl)amino]propoxy}-4,7-difluoro-3-ethyl-1-benzothien-2-yl)sulfonyl]amino}methyl)phosphonate

¹H NMR (500 MHz, CD₃OD) δ (ppm): 7.60 (t, 1H), 7.30 (d, 2H), 7.20 (d, 2H), 7.10 (d, 2H), 6.80 (d, 2H), 4.40 (t, 2H), 4.30 (t, 2H), 3.60 (m, 4H), 3.50 (d, 2H), 3.20 (q, 2H), 2.18 (m, 4H), 1.28 (t, 3H); MS m/z 909 (M+1).

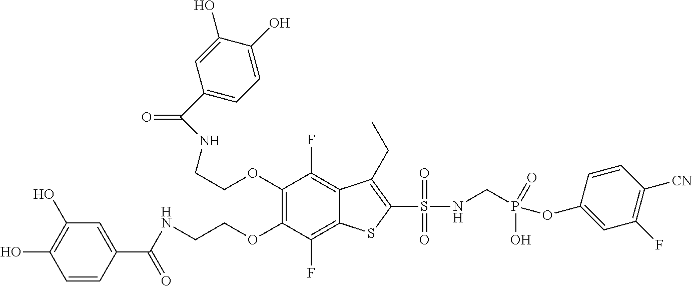

### 4-cyano-3-fluorophenyl hydrogen ({[(5,6-bis{2-[(3,4-dihydroxybenzoyl)amino]ethoxy}-4,7-difluoro-3-ethyl-1-benzothien-2-yl)sulfonyl]amino}methyl)phosphonate

¹H NMR (500 MHz, CD₃OD) δ (ppm): 7.60 (t, 1H), 7.30 (d, 2H), 7.20 (m, 2H), 7.10 (m, 2H), 6.80 (m, 2H), 4.40 (t, 2H), 4.30 (t, 2H), 3.70 (m, 4H), 3.45 (d, 2H), 3.14 (q, 2H), 1.20 (t, 3H); MS m/z 881 (M+1).

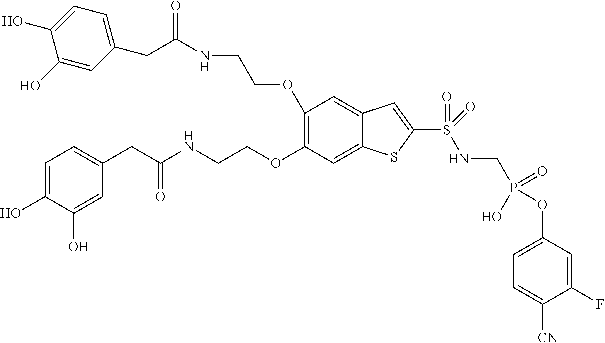

### 4-cyano-3-fluorophenyl hydrogen[({[5,6-bis(2-{[(3,4-dihydroxyphenyl)acetyl]amino}ethoxy)-1-benzothien-2-yl]sulfonyl}amino)methyl]phosphonate

¹H NMR (500 MHz, CD₃OD) δ (ppm): 7.8 (s, 1H), 7.5 (t, 1H), 7.4 (s, 1H), 7.35 (s, 1H), 7.2 (d, 1H), 7.15 (d, 1H), 6.8 (dd, 2H), 6.7 (m, 2H), 6.6 (t, 2H), 4.15 (t, 2H), 4.10 (t, 2H), 3.6 (m, 4H), 3.4 (s, 2H); MS m/z 845 (M+1).

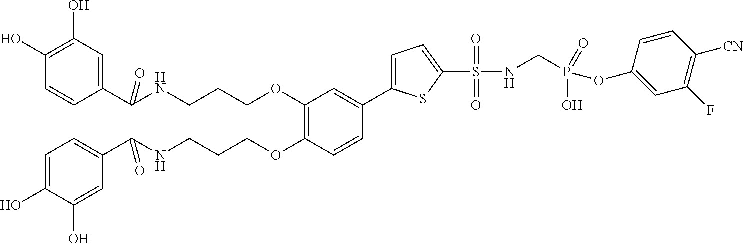

### 4-cyano-3-fluorophenyl hydrogen[({[5-(3,4-bis{3-[(3,4-dihydroxybenzoyl)amino]propoxy}phenyl)-2-thienyl]sulfonyl}amino)methyl]phosphonate

¹H NMR (500 MHz, CD₃OD) δ (ppm): 7.64 (m, 1H), 7.56 (d, 1H), 7.20 (m, 9H), 7.02 (d, 1H), 6.80 (d, 2H), 4.20 (m, 4H), 3.60 (m, 4H), 3.40 (d, 2H), 2.10 (m, 4H); MS m/z 871 (M+1).

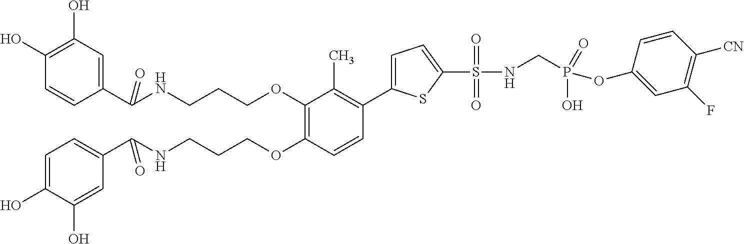

### 4-cyano-3-fluorophenyl hydrogen[({[5-(3,4-bis{3-[(3,4-dihydroxybenzoyl)amino]propoxy}-2-methylphenyl)-2-thienyl]sulfonyl}amino)methyl]phosphonate

¹H NMR (500 MHz, CD₃OD) δ (ppm): 7.70 (m, 2H), 7.60 (m, 2H), 7.30 (m, 2H), 7.22 (m, 2H), 7.10 (d, 1H), 7.05 (d, 1H), 6.98 (d, 1H), 6.80 (m, 2H), 4.14 (m, 4H), 3.62 (t, 2H), 3.60 (t, 2H), 3.30 (d, 2H), 2.10 (m, 4H); MS m/z 885 (M+1).

### Example 4

### Preparation of 4-Cyano-3-fluorophenyl hydrogen (5-{3,4-bis[3-(3,4-dihydroxybenzamido) propoxy]benzamido}benzo[b]thiophene-2-sulfonamido)methylphosphonate

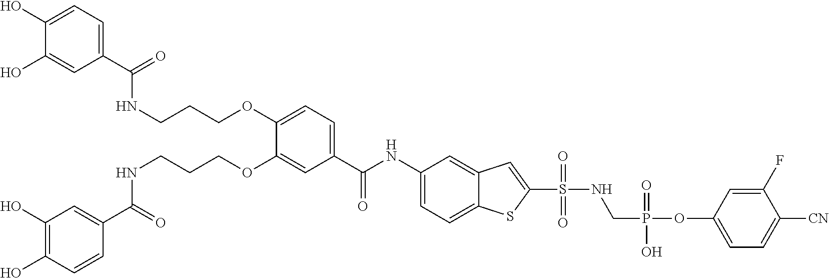

Step 1. To a stirred solution of 3,4-bis{3-[3,4-bis(methoxymethoxy)benzamido]propoxy}benzoic acid (120 mg, 0.17 mmol) in anhydrous DMF (3 mL) at room temperature under nitrogen were added DIPEA (44 μL, 0.25 mmol) and HATU (96 mg, 0.25 mmol). After 5 min, a solution of ammonium-3-fluoro-4-cyanophenyl-(5-aminobenzo[b]thiophene-2-sulfonamido)methyl-phosphonate (37 mg, 0.08 mmol) and DIPEA (30 μL, 0.17 mmol) in anhydrous DMF (1 mL) was added. The reaction mixture was stirred overnight, quenched with a little bit of methanol and water, and purified twice by preparative HPLC (Thermo, Aquasil C18, 250×21.2 mm, 5 μm; eluent MeOH/H₂O [both containing 0.05% HCO₂H], linear gradient 30/70→95/5 over 30 min), to afford the crude intermediate, which was used without further purification.

Step 2. To a stirred solution of the product of the prior step in methanol (5 mL) at room temperature under nitrogen was added a methanolic solution of 3N HCl (5 mL). The reaction mixture was stirred for 4 h, quenched with water, concentrated under reduced pressure, and purified twice by preparative HPLC (Thermo, Aquasil C18, 250×21.2 mm, 5 μm; eluent MeOH/H₂O [both containing 0.05% HCO₂H], linear gradient 30/70→95/5 and 30/70→80/20 over 30 min), to afford the product (2.8 mg) as a colorless sticky film.

¹H NMR (400 MHz, MeOH-d₄) δ (ppm): 8.31 (s, 1H), 7.87 (d, J=8.6 Hz, 1H), 7.82 (s, 1H), 7.77 (d, J=8.0 Hz, 1H), 7.68-7.52 (m, 3H), 7.45 (t, J=7.7 Hz, 1H), 7.27 (bs, 3H), 7.19 (bd, J=8.2 Hz, 2H), 7.12 (bd, J=11.2 Hz, 1H), 7.06 (bd, J=8.4 Hz, 1H), 7.02 (bd, J=8.6 Hz, 1H), 6.77 (dd, J=7.5 Hz, 2H), 6.78-6.68 (m, 1H), 4.24-4.08 (m, 4H), 3.64-3.46 (m, 4H), one CH₂ is hidden by methanol signals, 2.14-1.98 (m, 4H).

Utilizing the foregoing procedure described throughout the specification, the following additional compounds were prepared:

### Example 5

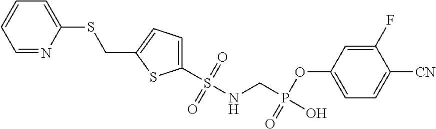

4-cyano-3-fluorophenyl hydrogen (5-((pyridin-2-ylthio)methyl)thiophene-2-sulfonamido)-methylphosphonate

(DMSO-d6) δ (ppm): 8.48 (ddd, J=4.9, 2.0, 1.0, 1H); 7.71 (t, J=8.4, 1H); 7.66 (ddd, J=8.0, 7.2, 2.0, 1H); 7.34 (d, J=3.7, 1H); 7.32 (dt, J=8.0, 1.0, 1H); 7.28 (dd, J=11.5, 1.6, 1H); 7.15 (ddd, J=7.4, 4.9, 1.0, 1H); 7.12 (br s, 5H); 7.07 (dd, J=7.8, 2.1, 1H); 7.06 (d, J=3.7, 1H); 4.66 (s, 2H); 2.75 (d, J=13.1, 2H). LRMS: 498.0 (calc) 498.1 (found);

### Example 6

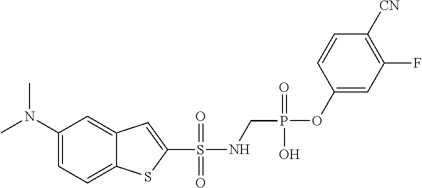

4-cyano-3-fluorophenyl hydrogen (5-(dimethylamino)benzo[b]thiophene-2-sulfonamido)-methylphosphonate;

¹H NMR (400 MHz, DMSO-d₆) δ (ppm): 7.77 (d, J=6.6, 1H); 7.69 (s, 1H); 7.58 (t, J=6.4, 1H); 7.24 (d, J=10.0, 1H); 7.14 (br s, 5H), 7.08 (d, J=6.8, 1H); 7.03 (d, J=6.8, 1H); 2.94 (s, 6H); 2.86 (d, J=10.4, 2H).

LRMS: 468.0 (calc), 468.0 (found)

### Example 7

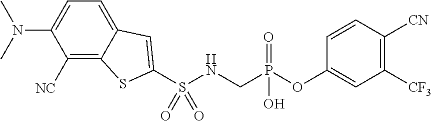

4-cyano-3-(trifluoromethyl)phenyl hydrogen (7-cyano-6-(dimethylamino)benzo[b]thiophene-2-sulfonamido)methylphosphonate

¹H NMR (400 MHz, DMSO-d₆) δ (ppm): 7.99-7.90 (m, 1H), 7.90 (d, J=9.2 Hz, 1H), 7.89 (d, J=8.0 Hz, 1H), 7.80 (s, 1H), 7.70 (s, 1H), 7.46 (d, J=8.0 Hz, 1H), 7.10 (d, J=9.2 Hz, 1H), 3.22 (s, 6H), 3.03 (dd, J=13.2, 4.8 Hz, 2H).

### Example 8

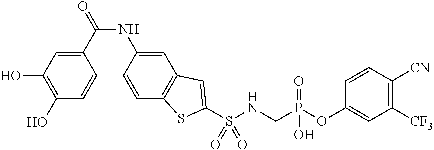

4-cyano-3-(trifluoromethyl)phenyl hydrogen (5-(3,4-dihydroxybenzamido)benzo[b]thiophene-2-sulfonamido)methylphosphonate

¹H NMR (400 MHz, DMSO-d₆) δ (ppm): 10.12 (s, 1H), 8.44 (d, J=1.6 Hz, 1H), 8.05-7.85 (m, 4H), 7.79 (dd, J=8.8, 2.0 Hz, 1H), 7.76 (d, J=2.0 Hz, 1H), 7.50 (dd, J=8.6, 2.0 Hz, 1H), ABX system (J_{A}=6.82, J_{B}=7.36, J_{X}=7.40, J_{AB}=8.2 Hz, J_{BX}=2.0 Hz, J_{AX}=0 Hz, 3H), 3.05 (d, J=12.3, 5.5 Hz, 2H). MS (m/z) 626.0 [M-H]⁺

### Example 9

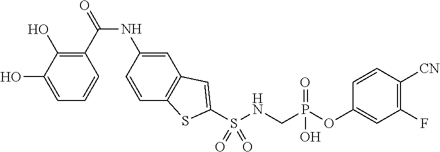

4-cyano-3-fluorophenyl hydrogen (5-(2,3-dihydroxybenzamido)benzo[b]thiophene-2-sulfonamido)methylphosphonate

¹H NMR (400 MHz, DMSO-d₆) δ (ppm): 11.61 (bs, 1H), 10.52 (s, 1H), ABX system (J_{A}=8.03, J_{B}=7.74, J_{X}=8.38, J_{AB}=8.8 Hz, J_{BX}=2.0 Hz, J_{AX}=0 Hz, 3H), 8.16 (bs, 1H), 7.98 (s, 1H), 7.74 (t, J=8.2 Hz, 1H), 7.45 (dd, J=8.0, 1.2 Hz, 1H), 7.29 (dd, J=11.4, 1.9 Hz, 1H), 7.11 (d, J=8.6 Hz, 1H), 6.98 (dd, J=7.5, 1.5 Hz, 1H), 6.77 (t, J=7.8 Hz, 1H), 3.20-3.09 (m, 2H). MS (m/z): 576.0 [M-H]⁺.

### Example 10

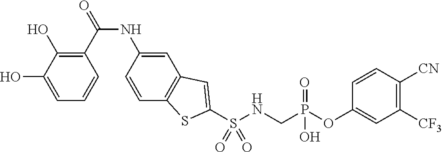

4-cyano-3-(trifluoromethyl)phenyl hydrogen (5-(2,3-dihydroxybenzamido)benzo[b]thiophene-2-sulfonamido)methylphosphonate

¹H NMR (400 MHz, CD₃OD) δ (ppm): 8.52 (s, 1H), 8.35 (bs, 1H), 7.92-7.83 (m, 2H), 7.79-7.67 (m, 3H), 7.51-7.43 (m, 2H), 6.97 (d, J=7.8 Hz, 1H), 6.76 (t, J=8.0 Hz, 1H), one CH₂ is masked by methanol. MS (m/z): 626.0 [M-H]⁺.

### Example 11

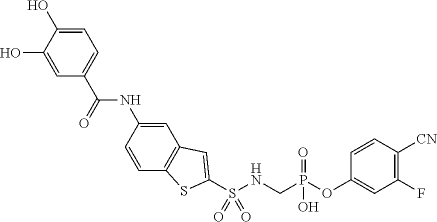

4-cyano-3-fluorophenyl hydrogen (5-(3,4-dihydroxybenzamido)benzo[b]thiophene-2-sulfonamido)methylphosphonate

¹H NMR (400 MHz, MeOH-d₄) δ (ppm): 8.56 (s, 1H), ABX system (J_{A}=7.90, J_{B}=7.79, J_{X}=8.32, J_{AB}=8.8 Hz, J_{BX}=2.1 Hz, J_{AX}=0 Hz, 3H), 7.86 (s, 1H), 7.51 (dd, J=8.1 Hz, 1H), A′B′X′ system (J_{A′}=6.88, J_{B′}=7.41, J_{X′}=7.44, J_{A′B′}=8.2 Hz, J_{B′X′}=2.2 Hz, J_{A′X′}=0 Hz, 3H), 7.17 (dd, J=11.0, 2.2 Hz, 1H), 7.08 (d, J=8.4 Hz, 1H), one CH₂ is masked by methanol. MS (m/z): 576.0 [M-H]⁺. MS (m/z): 576.0 [M-H]⁺.

### Example 12

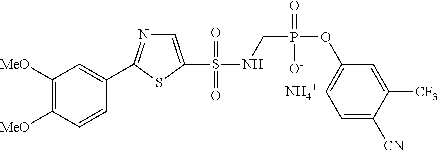

Ammounium 4-cyano-3-(trifluoromethyl)phenyl (2-(3,4-dimethoxyphenyl)thiazole-5-sulfonamido)methylphosphonate

δ (ppm, DMSO-d₆): 8.14 (d, J=0.8 Hz, 1H), 7.92 (d, J=8.6 Hz, 1H), 7.76 (d, J=2.2 Hz, 1H), 7.50 (m, 2H), 7.45 (d, J=2.0 Hz, 1H), 7.18 (m, 5H), 7.07 (d, J=8.6 Hz, 1H), 3.84 and 3.83 (2s, 6H), 2.91 (d, 2JHP=12.7 Hz, 2H).

MS: 562 (cal.) 561 (found, M−1).

### Example 13

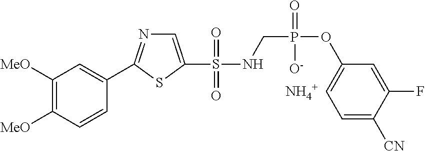

Ammonium 4-cyano-3-fluorophenyl (2-(3,4-dimethoxyphenyl)thiazole-5-sulfonamido)methylphosphonate

δ (ppm, DMSO-d₆): 8.14 (d, J=0.9 Hz, 1H), 7.65 (t, J=8.2 Hz, 1H), 7.52 (d, J=2.0 Hz, 1H), 7.50 (d, J=2.1 Hz, 1H), 7.46 (d, J=2.1 Hz, 1H), 7.29 (d, J=2.0 Hz, 1H), 7.26 (d, J=2.0 Hz, 1H), 7.17 (m, 5H), 3.85 and 3.83 (2s, 6H), 2.89 (d, 2JHP=12.9 Hz, 2H).

MS: 512 (cal.) 511 (found, M−1).

### Example 14

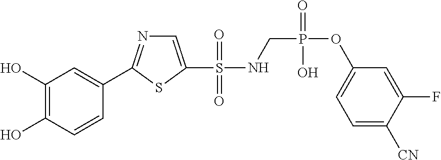

4-cyano-3-fluorophenyl hydrogen (2-(3,4-dihydroxyphenyl)thiazole-5-sulfonamido)methylphosphonate

δ (ppm, DMSO-d₆): 8.10 (s, 1H), 7.64 (t, J=8.2 Hz, 1H), 7.38 (d, J=2.1 Hz, 1H), 7.31 (dd, J=8.2 Hz, 2.1 Hz, 1H), 7.22 (d, J=10.8 Hz, 1H), 7.16 (d, J=8.4 Hz, 1H), 6.85 (d, J=8.2 Hz, 1H), 3.42 (d, 2JHP=12.9 Hz, 2H).

MS: 485 (cal.) 484 (found, M−1).

### Example 15

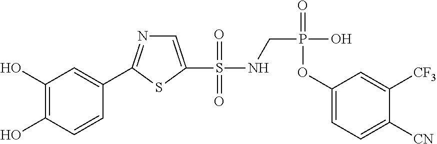

4-cyano-3-(trifluoromethyl)phenyl hydrogen (2-(3,4-dihydroxyphenyl)thiazole-5-sulfonamido)methylphosphonate

δ (ppm, DMSO-d₆): 8.10 (s, 1H), 7.89 (d, J=8.4 Hz, 1H), 7.75 (s, 1H), 7.58 (d, J=8.2 Hz, 1H), 7.37 (d, J=2.1 Hz, 1H), 7.30 (dd, J=8.2 Hz, 2.1 Hz, 1H), 6.84 (d, J=8.2 Hz, 1H), 3.42 (d, 2JHP=12.5 Hz, 2H).

MS: 535 (cal.) 534 (found, M−1).

### Example 16

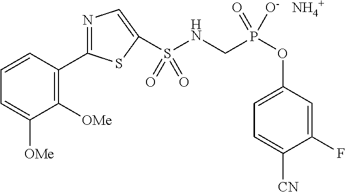

Ammonium 4-cyano-3-fluorophenyl (2-(2,3-dimethoxyphenyl)thiazole-5-sulfonamido)methylphosphonate

δ (ppm, DMSO-d₆): 8.24 (d, J=0.6 Hz, 1H, collapsed to singlet upon D2O exchange), 7.81 (m, 1H), 7.63 (dd, J=8.4 Hz, 8.4 Hz, 1H), 7.00-7.28 (m, 7H, collapsed to 2m with 4H upon D2O exchange), 3.93 (s, 3H), 3.89 (s, 3H), 2.88 (d, J=12.9 Hz).

MS: 512 (cal.) 511 (found, M−1).

### Example 17

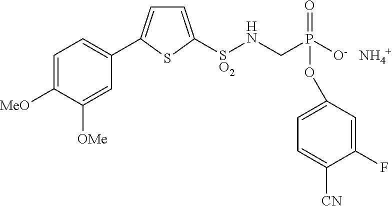

Ammonium 4-cyano-3-fluorophenyl (5-(3,4-dimethoxyphenyl)thiophene-2-sulfonamido)methylphosphonate

δ (ppm, DMSO-d₆, d₂O exchange): 7.66 (dd, J=8.2, 8.4 Hz, 1H), 7.49 (d, J=3.9 Hz, 1H), 7.41 (d, J=3.9 Hz, 1H), 7.24 (dd, J=1.5, 11.6 Hz, 1H), 7.18 (m, 1H), 7.08 (m, 1H), 6.99 (m, 1H), 3.81 (s, 3H), 3.77 (s, 3H), 2.87 (d, J=13.3 Hz, 2H).

MS: 511 (cal.) 510 (found, M−1).

### Example 18

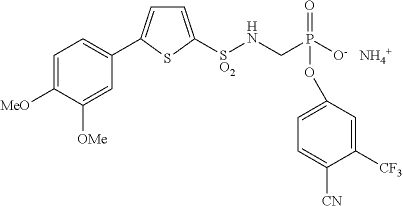

Ammonium 4-cyano-3-(trifluoromethyl)phenyl (5-(3,4-dimethoxyphenyl)thiophene-2-sulfonamido)methylphosphonate

δ (ppm, DMSO-d₆, d₂O exchange): 7.93 (d, J=8.6 Hz, 1H), 7.72 (d, J=2.0 Hz, 1H), 7.49 (m, 2H), 7.40 (d, J=3.9 Hz, 1H), 7.16-7.19 (m, 2H), 6.99 (m, 1H), 3.81 (s, 3H), 3.77 (s, 3H), 2.89 (d, J=13.1 Hz, 2H).

MS: 561 (cal.) 560 (found, M−1).

### Example 19

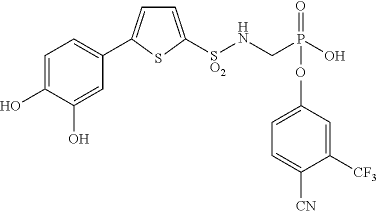

4-cyano-3-(trifluoromethyl)phenyl hydrogen (5-(3,4-dihydroxyphenyl)thiophene-2-sulfonamido)methylphosphonate

δ (ppm, DMSO-d₆): 7.89 (m, 1H), 7.79 (dd, J=8.2, 8.4 Hz, 1H), 7.48 (d, J=3.9 Hz, 1H), 7.30 (dd, J=1.8, 11.3 Hz, 1H), 7.23 (d, J=3.9 Hz, 1H), 7.14 (m, 1H), 7.00 (d, J=2.3 Hz, 1H), 6.95 (dd, J=2.3, 8.1 Hz, 1H), 6.75 (d, J=8.2 Hz, 1H), 3.08 (m, 2H).

MS: 534 (cal.) 533 (found, M−1).

### Example 20

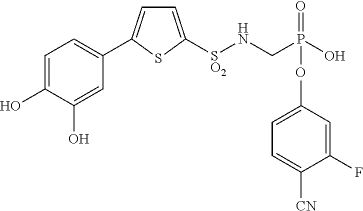

4-cyano-3-fluorophenyl hydrogen (5-(3,4-dihydroxyphenyl)thiophene-2-sulfonamido)methylphosphonate

δ (ppm, DMSO-d₆): 8.06 (d, J=8.2 Hz, 1H), 7.94 (m, 1H), 7.78 (d, J=2.0 Hz, 1H), 7.58 (d, J=8.2 Hz, 1H), 7.49 (m, 1H), 7.24 (d, J=3.9 Hz, 1H), 7.01 (d, J=2.2 Hz, 1H), 6.95 (dd, J=2.3, 8.2 Hz, 1H), 6.76 (d, J=8.2 Hz, 1H), 3.20 (m, 2H).

MS: 484 (cal.) 483 (found, M−1).

### Example 21

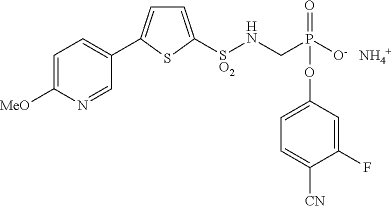

Ammonium 4-cyano-3-fluorophenyl (5-(6-methoxypyridin-3-yl)thiophene-2-sulfonamido)methylphosphonate

δ (ppm, DMSO-d₆): 8.50 (m, 1H), 8.00 (m, 1H), 7.67 (dd, J=8.4, 8.4 Hz, 1H), 7.54 (dd, J=1.2, 3.9 Hz, 1H), 7.47 (dd, J=1.2, 3.9 Hz, 1H), 7.28 (m, 1H), 7.02-7.14 (bs, 5H), 6.90 (m, 1H), 3.89 (s, 3H), 2.84 (d, J=13.1, 2H).

MS: 482 (cal.) 481 (found, M−1).

### Example 22

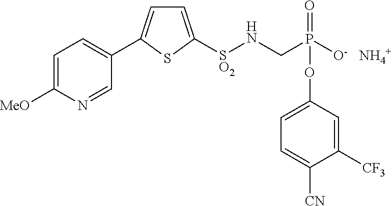

Ammonium 4-cyano-3-(trifluoromethyl)phenyl (5-(6-methoxypyridin-3-yl)thiophene-2-sulfonamido)methylphosphonate

δ (ppm, DMSO-d₆): 8.49 (m, 1H), 8.00 (dd, J=2.6, 9.0 Hz, 1H), 7.94 (d, J=8.6 Hz, 1H), 7.76 (d, J=2.0 Hz, 1H), 7.54 (m, 1H), 7.51 (m, 1H), 7.46 (m, 1H), 6.96 (bs, 3H), 6.89 (m, 1H), 3.89 (s, 3H), 2.86 (d, J=13.1, 2H)

MS: 532 (cal.) 531 (found, M−1).

### Example 23

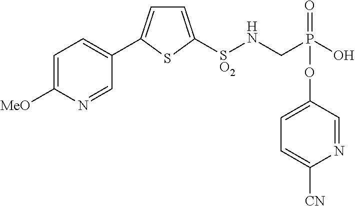

6-cyanopyridin-3-yl hydrogen (5-(6-methoxypyridin-3-yl)thiophene-2-sulfonamido)methylphosphonate

δ (ppm, DMSO-d₆): 8.49 (m, 1H), 8.47 (d, J=2.5 Hz, 1H), 7.99 (dd, J=2.6, 8.7 Hz, 1H), 7.91 (d, J=8.4 Hz, 1H), 7.77-7.74 (m, 1H), 7.66 (d, J=3.7 Hz, 1H), 7.47 (d, J=3.9 Hz, 1H), 6.90 (dd, J=0.8, 8.6 Hz, 1H), 3.88 (s, 3H), 3.07 (d, J=12.7 Hz, 2H).

MS: 466 (cal.) 465 (found, M−1).

### Example 24

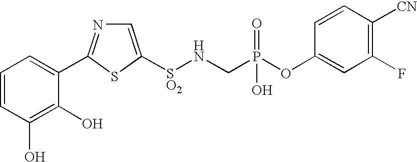

4-cyano-3-fluorophenyl hydrogen (2-(2,3-dihydroxyphenyl)thiazole-5-sulfonamido)methylphosphonate

δ (ppm, DMSO-d₆): 10.60 (s, 1H), 9.97 (s, 1H), 8.16-8.28 (m, 2H), 7.80 (t, J=8.3 Hz, 1H), 7.61 (m, 1H), 7.30 (m, 1H), 7.16 (d, J=8.6 Hz, 1H), 6.93 (dd, J=1.6, 7.8 Hz, 1H), 6.80 (dd, 7.8, 7.8 Hz, 1H), 3.19 (m, 2H).

MS: 485 (cal.) 484 (found, M−1).

### Example 25

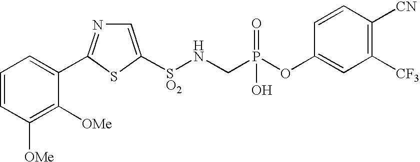

4-cyano-3-(trifluoromethyl)phenyl hydrogen (2-(2,3-dimethoxyphenyl)thiazole-5-sulfonamido)methylphosphonate

δ (ppm, DMSO-d₆): 8.32 (m, 1H), 8.29 (s, 1H), 8.05 (d, J=8.4 Hz, 1H), 7.80 (m, 1H), 7.75 (m, 1H), 7.57 (m, 1H), 7.19-7.25 (m, 1H), 3.93 (s, 3H), 3.89 (s, 3H), 3.23 (dd, J=12.3, 12.5 Hz, 2H).

MS: 563 (cal.) 562 (found, M−1).

### Example 26

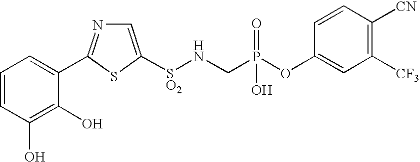

4-cyano-3-(trifluoromethyl)phenyl hydrogen (2-(2,3-dihydroxyphenyl)thiazole-5-sulfonamido)methylphosphonate

δ (ppm, DMSO-d₆): 8.22 (s, 1H), 7.85 (d, J=8.4 Hz, 1H), 7.72 (s, 1H), 7.56 (d, J=8.0 Hz, 1H), 7.45 (d, J=7.8 Hz, 1H), 6.92 (d, J=7.8 Hz, 1H), 6.79 (dd, J=7.8, 8.0 Hz, 1H), 3.46 (d, J=12.3 Hz, 2H).

MS: 535 (cal.) 534 (found, M−1).

### Example 27

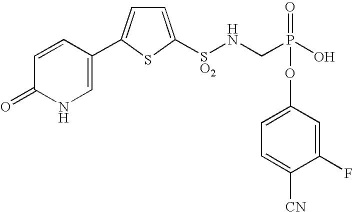

4-cyano-3-fluorophenyl hydrogen (5-(6-oxo-1,6-dihydropyridin-3-yl)thiophene-2-sulfonamido)methylphosphonate

δ (ppm, DMSO-d₆): 8.50 (s, 1H), 7.86-7.89 (m, 1H), 7.79 (d, J=2.0 Hz, 1H), 7.61 (dd, J=7.8, 8.0 Hz, 1H), 7.53 (dd, J=1.0, 3.9 Hz, 1H), 7.24 (dd, J=1.2, 3.9 Hz, 1H), 7.17 (m, 1H), 6.62 (dd, J=0.8, 9.4 Hz, 1H), 3.22 (d, J=13.5 Hz, 2H).

MS: 469 (cal.) 468 (found, M−1).

### Example 28

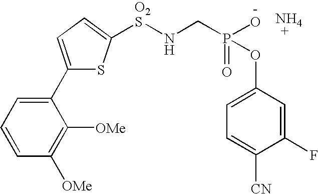

Ammonium 4-cyano-3-fluorophenyl (5-(2,3-dimethoxyphenyl)thiophene-2-sulfonamido)methylphosphonate

δ (ppm, DMSO-d₆): 7.66 (m, 2H), 7.52 (d, J=3.7 Hz, 1H), 7.44 (m, 1H), 7.29 (m, 1H), 7.02-7.18 (bs, 7H), 3.86 (s, 3H), 3.79 (s, 3H), 2.69 (d, J=13.1 Hz, 2H).

MS: 511 (cal.) 510 (found, M−1).

### Example 29

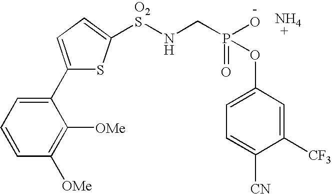

Ammonium 4-cyano-3-(trifluoromethyl)phenyl (5-(2,3-dimethoxyphenyl)thiophene-2-sulfonamido)methylphosphonate

δ (ppm, DMSO-d₆): 7.94 (d, J=8.4 Hz, 1H), 7.78 (d, J=2.3 Hz, 1H), 7.62 (d, J=4.1 Hz, 1H), 7.49-7.52 (m, 2H), 7.43 (dd, J=1.5, 7.9 Hz, 1H), 7.13 (dd, J=7.9, 7.9 Hz, 1H), 7.06 (dd, J=1.4, 8.4 Hz, 1H), 6.94 (bs, 5H), 3.85 (s, 3H). 3.74 (s, 3H), 2.85 (d, J=12.9 Hz, 2H).

MS: 561 (cal.) 560 (found, M−1).

### Example 30

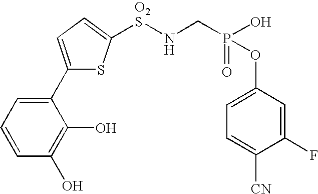

4-cyano-3-fluorophenyl hydrogen (5-(2,3-dihydroxyphenyl)thiophene-2-sulfonamido)methylphosphonate

δ (ppm, DMSO-d₆): 9.77 (s, 1H), 9.62 (s, 1H), 7.76 (dd, J=8.2, 8.4 Hz, 1H), 7.64 (bs, 1H), 7.56 (d, J=4.1 Hz, 1H), 7.51 (d, J=4.1 Hz, 1H), 7.31 (dd, J=2.0, 11.5 Hz, 1H), 7.20 (dd, J=1.4, 7.8 Hz, 1H), 7.13 (dd, J=1.9, 8.7 Hz, 1H), 6.78 (dd, J=1.3, 7.8 Hz, 1H), 6.70 (dd, J=9.6, 7.8 Hz, 1H), 2.99 (m, 2H).

MS: 484 (cal.) 483 (found, M−1).

### Example 31

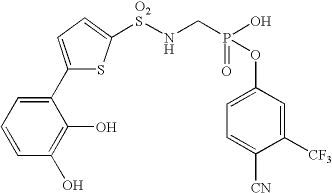

4-cyano-3-(trifluoromethyl)phenyl hydrogen (5-(2,3-dihydroxyphenyl)thiophene-2-sulfonamido)methylphosphonate

δ (ppm, DMSO-d₆): 9.78 (bs, 1H), 9.65 (bs, 1H), 8.10 (d, J=8.6 Hz, 1H), 8.05 (bs, 1H), 7.78 (d, J=2.2 Hz, 1H), 7.60 (dd, J=2.0, 8.6 Hz, 1H), 7.56 (d, J=4.1 Hz, 1H), 7.53 (d, J=4.1, Hz, 1H), 7.20 (dd, J=1.4, 8.0 Hz, 1H), 6.77 (dd, J=1.6, 7.8 Hz, 1H), 6.70 (dd, J=7.8, 8.0 Hz, 1H), 3.18 (m, 2H).

MS: 534 (cal.) 533 (found, M−1).

### Example 32

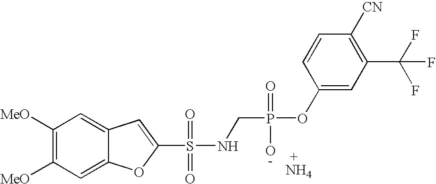

Ammonium 4-cyano-3-(trifluoromethyl)phenyl (5,6-dimethoxybenzofuran-2-sulfonamido)methylphosphonate

δ (ppm, DMSO-d₆): 7.89 (d, J=8.6 Hz, 1H), 7.71 (d, J=2.1, 1H), 7.46 (dd, J=8.4, 2.1 Hz, 1H), 7.33 (d, J=0.7 Hz, 1H), 7.26 (s, 1H), 7.40-7.00 [m, 5H, includes peak at 7.16 (s, 1H)], 3.82 and 3.78 (2s, 6H), 2.95 (d, 2JHP=13.5 Hz, 2H).

MS: 519 (cal.) 518 (found, M−1).

### Example 33

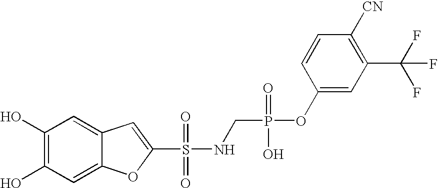

4-cyano-3-(trifluoromethyl)phenyl hydrogen (5,6-dihydroxybenzofuran-2-sulfonamido)methylphosphonate

δ (ppm, DMSO-d₆): 7.78 (d, J=8.4 Hz, 1H), 7.69 (d, J=1.8, 1H), 7.46 (dd, J=8.8, 1.6 Hz, 1H), 7.22 (d, J=0.8 Hz, 1H), 6.97 (s, 1H), 6.89 (d, J=0.8 Hz, 1H), 3.28 (d, 2JHP=12.9 Hz, 2H).

MS: 492 (cal.) 492 (found, M−1).

### Example 34

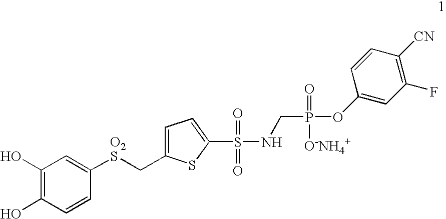

Ammonium 4-cyano-3-fluorophenyl (5-((3,4-dihydroxyphenylsulfonyl)methyl)thiophene-2-sulfonamido)methylphosphonate

1: ¹H NMR (DMSO-d₆) δ (ppm): 8.10 (m, 1H); 7.53 (br s, 4H); 7.35-7.30 (m, 2H); 7.14 (d, J=8.2, 1H); 6.92-6.80 (m, 4H); 4.84 (s, 2H); 2.77 (d, J=12.1, 2H).

LRMS (Neg. Scan): 561.0 (calc) 561.0 (found).

### Biological Assays

### Enzyme Activity: Determination of IC₅₀.

The Class A and C enzyme activities were measured in the presence of the test inhibitor in spectrophotometric assay against the commercially available substrate, nitrocefin, while the Class D enzyme activity was determined with CCF2/FA™ (Invitrogen) as substrate in a fluorometric assay. The enzymes, TEM-1, Amp C (P. aeruginosa), Amp C (A. baumanii), P99, and Oxa-40, as well as both substrates, were dissolved in 100 mM KH₂PO₄ buffer (pH 7). For the spectrophotometric assay, the buffer also contains 0.005% BSA and for the fluorometric assay, it also contains 0.005% tween-20. The test inhibitor was dissolved in DMSO and diluted 1:20 in the assay, resulting in a final concentration range of 50 uM to 0.0002 uM. In a 96-well microplate, the test inhibitor was incubated with the beta-lactamase enzyme for 40 minutes at ambient temperature, the substrate solution was added, and the incubation continued for another 40 minutes. The spectrophotometric reaction is quenched by the addition of 2.5 N acetic acid, resulting in a final concentration of 0.5N, and the absorbance at 492 nm was measured. The fluorometric assay reaction was quenched with 1.5% SDS, resulting in a final concentration of 0.3% SDS, and the fluorescence was measured at excitation 400 nm, emission 460 nm. The IC₅₀ was determined from semi logarithmic plots of enzyme inhibition versus inhibitor concentration, with a curve generated using a 4-parameter fit. Representative results using the test inhibitors of the invention showing the inhibition of Class A, C, and D beta-lactamases are shown in Tables 1 and 2.

TABLE 1
Beta-lactamase Inhibitory Activity of Sulfonamidomethylphosphonate Derivatives

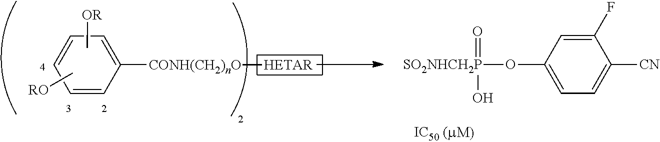

| Cmpd. | HETAR | R | n | Tem-1^{a} | Amp C^{b} | Amp C^{c} | P99^{d} | Pxa-40^{e} |
|---|---|---|---|---|---|---|---|---|
| 1 | 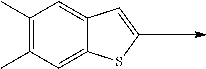 | 3,4-Ac | 2 | 0.1 | 0.01 | 0.04 | 0.002 | 0.03 |
| 2 | 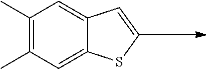 | 3,4-H | 2 | 0.2 | 0.01 | 0.07 | 0.002 | 0.03 |
| 3 | 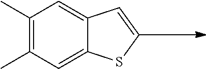 | 2,3-Ac | 2 | 0.5 | 0.03 | — | 0.002 | 0.1 |
| 4 | 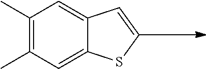 | 2,3-H | 2 | 0.7 | 0.01 | — | 0.002 | 0.05 |
| 5 | 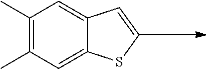 | 3,4-Ac | 3 | 0.1 | 0.009 | 0.04 | 0.0007 | 0.02 |
| 6 | 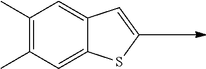 | 3,4-H | 3 | 0.08 | 0.005 | 0.02 | 0.0005 | 0.02 |
| 7 | 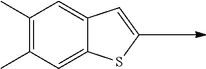 | — | 2 | 0.1 | 0.009 | 0.02 | 0.0005 | 0.03 |
| 8 | 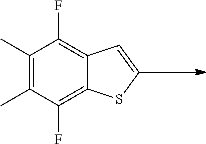 | 3,4-H | 2 | 0.05 | 0.002 | 0.007 | 0.003 | 0.03 |
| 9 | 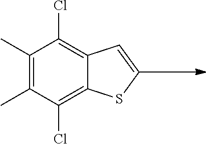 | 3,4-H | 2 | 0.1 | 0.005 | 0.009 | 0.003 | 0.02 |
| 10 | 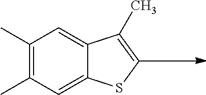 | 3,4-H | 3 | 0.1 | 0.004 | 0.03 | 0.0005 | 0.06 |
| 11 | 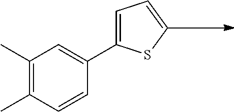 | 3,4-H | 3 | 0.4 | 0.01 | 0.05 | 0.003 | 0.03 |
| 12 | 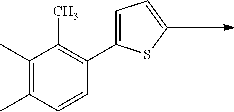 | 3,4-H | 3 | 0.6 | 0.02 | 0.03 | — | 0.03 |
| 13 | 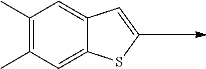 | 3,4-H | 4 | 0.1 | 0.004 | 0.01 | 0.0006 | 0.03 |
| 14 | 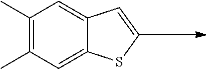 | 3,4-CH₃ | 2 | 0.3 | 0.001 | 0.03 | 0.0008 | 0.04 |

^{a}Class A beta-lactamase from E. coli PBR322
^{b}Class C beta-lactamase from Ps. aeruginosa, ATCC Genomic DNA
^{c}Class C beta-lactamase from A. baumanii, CL 6188
^{d}Class C beta-lactamase from E. cloacae, commercial source
^{e}Class D beta-lactamase from E. coli CL6190 in which OXA-40 gene was cloned from A. bau. CL6188

TABLE 2
Beta-lactamase Inhibitory Activity of Sulfonamidomethylphosphonate Derivatives

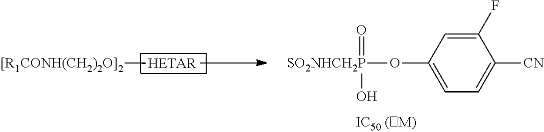

| Cmpd. | HETAR | R₁ | Tem-1^{a} | Amp C^{b} | Amp C^{c} | P99^{d} | Oxa-40^{e} |
|---|---|---|---|---|---|---|---|
| 1 | 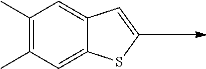 | 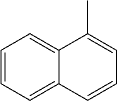 | 0.2 | 0.02 | 0.07 | 0.0009 | 0.09 |
| 2 | 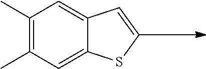 | 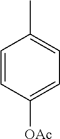 | 0.2 | 0.01 | 0.06 | 0.0007 | 0.02 |
| 3 | 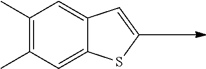 | 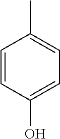 | 0.08 | 0.006 | 0.03 | 0.0003 | 0.03 |
| 4 | 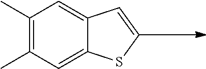 | 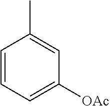 | 0.2 | 0.03 | 0.08 | 0.002 | 0.06 |
| 5 | 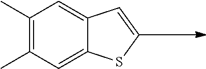 | 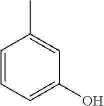 | 0.07 | 0.02 | 0.03 | 0.001 | 0.04 |
| 6 | 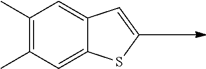 | 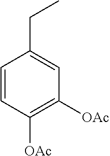 | 0.5 | 0.01 | 0.2 | 0.002 | 0.02 |
| 7 | 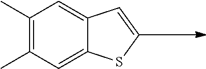 | 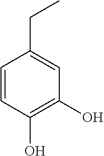 | 0.2 | 0.007 | 0.04 | 0.0009 | 0.006 |

^{a}Class A beta-lactamase from E. Coli PBR322
^{b}Class C beta-lactamase from Ps. aeruginosa, ATCC Genomic DNA
^{c}Class C beta-lactamase from A. baumanii, CL 6188
^{d}Class C beta-lactamase from E. cloacae, commercial source
^{e}Class D beta-lactamase from E. coli CL6190 in which OXA-40 gene was cloned from A. bau. CL6188

### Synergy Assay Protocol:

The assay determines the concentration of a β-lactamase inhibitor required to reduce the MIC of a β-lactam antibiotic by one-half, one-quarter, one-eighth, one-sixteenth and one-thirty-second against strains of bacteria normally resistant to the antibiotic in question. This is accomplished by titrating the inhibitor (BLI=beta-lactamase inhibitor) in a serial dilution across a microtiter plate while at the same time titrating the antibiotic (AB) in a serial dilution down the microtiter plate and then inoculating the plate with the bacterial strain in question and allowing the bacteria to grow up overnight. Each well in this microplate checkerboard contains a different combination of concentrations of the inhibitor and the antibiotic allowing a full determination of any synergy between the two.

### Bacterial Strain/Antibiotic Combinations:

CL 5701 (Pseudomonas aeruginosa; Pa AmpC)/Imipenem
MB 2646 (Enterobacter cloacae; P99)/Ceftazidime
CL 5513 (Klebsiella pneumoniae; SHV-5)/Ceftazidime
CL 6188 (Acinetobacter baumanii; Oxa40)/Imipenem
CL 6569 (Klebsiella pneumoniae; KPC-2)/Imipenem
CL 5761 (Klebsiella pneumoniae; KPC-3)/Imipenem
CLB 21648 (Acinetobacter baumanii; Ab AmpC)/Imipenem

### General Checkerboard Method:

1. All wells in rows B-H of MIC 2000 microtiter plates are filled with 100 μL of MHBII+1% DMSO (dimethyl sulfoxide).
2. All wells in row A of MIC 2000 microtiter plates are filled with 100 μL of 2×MHBII+2% DMSO.
3. 100 μL of 4× the final antibiotic concentration wanted is added to well A1 of the MIC 2000 plates.
4. 100 μL of 2× the final antibiotic concentration wanted is added to wells A2-A12 of the MIC 2000 plates.
5. 100 μL is serially diluted from row A to row G of each MIC 2000 plate.
6. 100 μL is removed from each well in row G of each MIC 2000 plate.
7. 100 μL of 2× the final inhibitor concentration wanted (in MHBII+1% DMSO) is added to all wells in column 1 of the microtiter plates.
8. 100 μL is serially diluted from column 1 to column 11 of each MIC 2000 plate.
9. 100 μL is removed from each well in column 11 of each MIC 2000 plate.
10. Plates are then inoculated with an overnight growth (in TSB) of the strain to be tested using an MIC 2000 inoculator.
11. Plates are left at 37° C. for about 20 hours and scored for growth by eye.

### Media (all are Sterilized by Autoclaving Prior to any Addition of DMSO):

| | | |
|---|---|---|
| MHBII + 1% DMSO | | |
| Mueller Hinton Broth type II cation adjusted (BBL ™) | 4.4 | g |
| DMSO | 2.0 | mL |
| Distilled water | 198.0 | mL |
| 2X MHBII + 2% DMSO | | |
| Mueller Hinton Broth type II cation adjusted (BBL ™) | 8.8 | g |
| DMSO | 4.0 | mL |
| Distilled water | 196.0 | mL |
| 1.02X MHBII | | |
| Mueller Hinton Broth type II cation adjusted (BBL ™) | 4.4 | g |
| Distilled water | 198.0 | mL |
| 1.1 X MHBII + 1% DMSO | | |
| Mueller Hinton Broth type II cation adjusted (BBL ™) | 4.4 | g |
| DMSO | 2.0 | mL |
| Distilled water | 178.0 | mL |
| TSB | | |
| Trypticase Soy Broth (BBL ™) prepared as directed on bottle. | | |

### Antibiotic Preparation:

Imipenem stocks are prepared at about 1280 μg/ml in 10 mM MOPS pH 7.0

They are stored at −80° C. in aliquots (the powder at −20° C.).

The true concentration is determined with hydroxylamine.

Dilutions are also prepared in 10 mM MOPS with a one day use.

Ceftazidime stocks are prepared at 10,240 μg/ml in Sorensen Buffer pH 7.0

They are stored at −20° C.

Dilutions are also prepared in Sorensen Buffer pH 7.0.

Sorensen Buffer

| | |
|---|---|
| 1/15 M Na₂HPO₄ | 61.1 mL |
| 1/15 M KH₂PO₄ | 38.9 mL |

Concentration of Antibiotics Used with Each Strain:

| | | Final Top | 4X | 2X Con- |
|---|---|---|---|---|
| Strain | Antibiotic | Concentration | Concentration | centration |
| CL 5701 | Imipenem | 40 μg/ml | 160 μg/ml | 80 μg/ml |
| MB 2646 | Ceftazidime | 256 μg/ml | 1024 μg/ml | 512 μg/ml |
| CL 5513 | Ceftazidime | 256 μg/ml | 1024 μg/ml | 512 μg/ml |
| CL 6188 | Imipenem | 256 μg/ml | 1024 μg/ml | 512 μg/ml |
| CL 6569 | Imipenem | 256 μg/ml | 1024 μg/ml | 512 μg/ml |
| CL 5761 | Imipenem | 16 μg/ml | 64 μg/ml | 32 μg/ml |
| CLB 21648 | Imipenem | 32 μg/ml | 128 μg/ml | 64 μg/ml |

### Inhibitor Preparation:

Test inhibitors are dissolved in 100% DMSO.

They are diluted (1:100) into 1.02×MHBII for a final concentration of MHBII+1% DMSO.
They are delivered usually either as 20 mM or 25.6 mg/ml in 100% DMSO and are tested at a final top concentration of either 100 μM or 128 μg/ml, but added to the plates as 2× these concentrations (i.e. 200 μM or 256 μg/ml) in MHBII+1% DMSO.
   Sulbactam is tested as a control inhibitor.

Stocks are prepared as 2560 μg/ml in sterile distilled water and stored at −20° C.

It is diluted to the appropriate concentration for each strain in 1.1×MHBII+1% DMSO.

This concentration is 2× the final top concentration at which it is tested.

Sulbactam Concentration

| Strain | Final | 2X |
|---|---|---|
| CL 5701 | 128 μg/ml | 256 μg/ml |
| MB 2646 | 128 μg/ml | 256 μg/ml |
| CL 5513 | 128 μg/ml | 256 μg/ml |
| CL 6188 | 32 μg/ml | 64 μg/ml |
| CL 6569 | 128 μg/ml | 256 μg/ml |
| CL 5761 | 128 μg/ml | 256 μg/ml |
| CLB 21648 | 8 μg/ml | 16 μg/ml |

### Inocula for BLI Checkerboards:

Brain Heart Infusion (BHI) Slants are good for 1 month at 4° C.

A single colony from a fresh streak (from a frozen vial) onto a BHI plate is used to make the slants.

Strains are grown in 2 ml of TSB in a snap cap 14 ml tube for 18 hrs at 37° C. at 220 rpm.

After 18 hrs tubes are placed on ice until ready to use.

Inocula are based on absorbance of a 1:10 dilution in TSB.
400 μL of overnight growth is added to 39.6 ml of 0.85% saline if the absorbance is as follows (using a Beckman DU-600) set at λ 600:

| Strain | Expected Absorbance |
|---|---|
| CL 5701 | 0.66 |
| MB 2646 | 0.62 |
| CL 5513 | 0.66 |
| CL 6188 | 0.70 |
| CL 6569 | 0.66 |
| CL 5761 | 0.66 |
| CLB 21648 | 0.70 |

If absorbance is not close to the expected value than the following correction is made.

All corrections are made at 100 μL increments. This is determined by multiplying the absorbance desired by 400 and dividing by the absorbance obtained and rounding to the closest 100 μL increment.

For example:

if CL 6188 absorbance is 0.8023 then:

$$\frac{( 0.7 ) \times ( 400 )}{( 0.8023 )} = 340$$

Therefore, add 300 μL overnight culture to 39.7 ml of 0.85% saline

### Reading of Plates:

Plates are scored for growth in each well. End points (MICs) where there is no growth in each row are determined and the concentrations of AB and the test BLI at each of these growth negative wells are then used to determine levels of synergy. Below is a checkerboard scoring/data grid. The final concentration of the antibiotic in each row is shown down the side of the checkerboard diagram and the final concentration of the inhibitor in each column is shown across the top of the checkerboard diagram.

Plate #1 CL5701 (Pseudomonas) MHBII + 1% DMSO Imipenem vs. Test BLI

| | | Test BLI | | | | | | | | | | | |
|---|---|---|---|---|---|---|---|---|---|---|---|---|---|
| | | 1 | 2 | 3 | 4 | 5 | 6 | 7 | 8 | 9 | 10 | 11 | 12 |
| Imipenem | Conc (per ml) | 100 μM | 50 | 25 | 12.5 | 6.25 | 3.125 | 1.563 | 0.782 | 0.391 | 0.196 | 0.098 | 0 |
| A | 40 μg | | | | | | | | | | | | |
| B | 20 | | | | | | | | | | | | |
| C | 10 | | | | | | | | | | | | |
| D | 5 | | | | | | | | | | | | |
| E | 2.5 | | | | | | | | | | | | |
| F | 1.25 | | | | | | | | | | | | |
| G | 0.625 | | | | | | | | | | | | |
| H | 0 | | | | | | | | | | | | GC |

Representative results using the test inhibitors of the invention showing the ability to synergize imipenem against resistant strains of Pseudomonas aeruginosa and Acinetobacter baumanii are shown in Tables 3 and 4.

TABLE 3
Synergy of Imipenem by Sulfonamidomethylphosphonate Derivatives Against Pseudomonas
aeruginosa, CL 5701, and Acinetobacter Baumanii, CL 6188

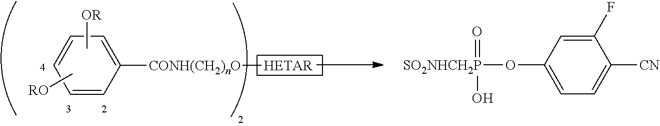

| | | | | Conc. (μM) to | Conc. (μM) to |
|---|---|---|---|---|---|
| | | | | achieve 4X synergy^{a} | achieve 4X synergy^{b} |
| Cmpd. | HETAR | R | n | Ps. Ar. | A. Baum. |
| 1 | 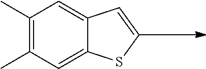 | 3,4-Ac | 2 | 6.25 | 25 |
| 2 | 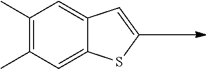 | 3,4-H | 2 | 25 | 12.5 |
| 3 | 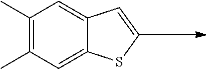 | 3,4-Ac | 3 | 6.25 | 3.13 |
| 4 | 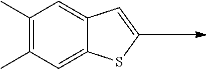 | 3,4-H | 3 | 25 | 1.56 |
| 5 | 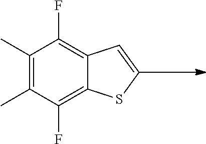 | 3,4-H | 2 | 25 | 0.78 |
| 6 | 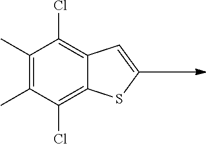 | 3,4-H | 2 | 6.25 | 3.13 |
| 7 | 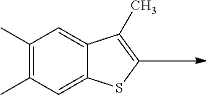 | 3,4-H | 3 | 12.5 | 50 |
| 8 | 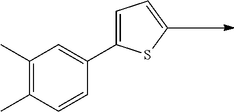 | 3,4-H | 3 | 12.5 | 12.5 |
| 9 | 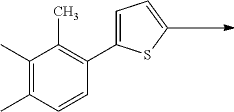 | 3,4-H | 3 | 6.26 | 12.5 |

^{a}Imipenem MIC = 20 μg/mL.
^{b}Imipenem MIC =128 μg/mL.

Representative results using the test inhibitors of the invention showing the ability to synergize imipenem against resistant strains of Pseudomonas aeruginosa and Acinetobacter baumanii are compared in Table 4 to the compounds that comprise the invention disclosed in the co-pending application. As seen in the table, the ammonium compounds of the co-pending application exhibit the ability to only synergize the antibacterial activity of imipenem against the resistant strain of Pseudomonas aeruginosa and not Acinetobacter baumanii. In contrast, the compounds of this invention exhibit the ability to synergize the antibacterial activity of imipenem against both resistant strains of Pseudomonas aeruginosa and Acinetobacter baumanii.

TABLE 4
Comparative Synergy of Imipenem by Sulfonamidomethylphosphonate Derivatives Against
Pseudomonas aeruginosa, CL 5701, and Acinetobacter baumanii, CL 1688

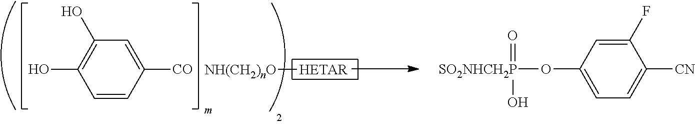

| | | | | Conc. (μM) to | Conc. (μM) to |
|---|---|---|---|---|---|
| | | | | achieve 4X synergy^{a} | achieve 4X synergy^{b} |
| Cmpd. | HETAR | m | n | Ps. Ar. | A. Baum. |
| 1* | 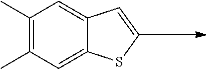 | 0 | 2 | 3.13 | >100 |
| 2 | 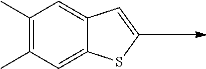 | 1 | 2 | 25 | 12.5 |
| 3* | 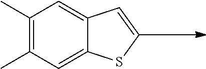 | 0 | 3 | 12.5 | >100 |
| 4 | 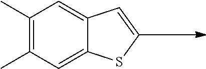 | 1 | 3 | 25 | 1.56 |
| 5* | 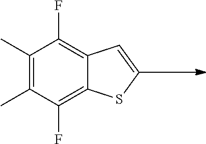 | 0 | 2 | 1.56 | >100 |
| 6 | 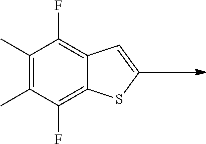 | 1 | 2 | 25 | 0.78 |
| 7* | 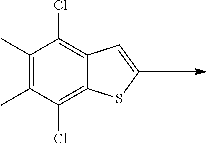 | 0 | 2 | 3.13 | >100 |
| 8 | 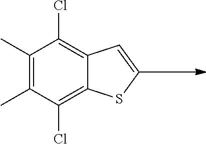 | 1 | 2 | 6.25 | 3.13 |
| 9* | 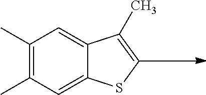 | 0 | 3 | 3.13 | >100 |
| 10 | 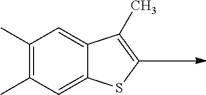 | 1 | 3 | 12.5 | 50 |
| 11* | 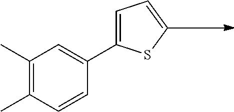 | 0 | 3 | 25 | >100 |
| 12 | 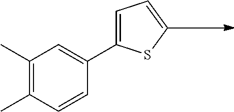 | 1 | 3 | 12.5 | 12.5 |
| 13* | 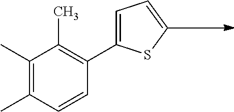 | 0 | 3 | 12.5 | >100 |
| 14 | 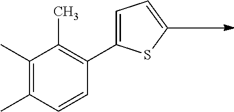 | 1 | 3 | 6.25 | 12.5 |

^{a}Imipenem MIC = 20 μg/mL.
^{b}Imipenem MIC = 128 μg/mL.
*Comparator compounds from Case 21841.

What is claimed is:

1. A compound of Formula I: 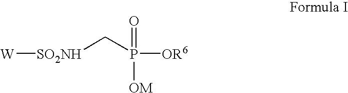
or a pro-drug or pharmaceutically acceptable salt thereof, wherein:
W represents: 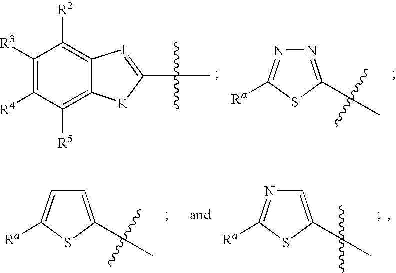
R^{a} represents: (CH₂)ₙKR^{aa}, or R^{aa};
R^{aa} represents: 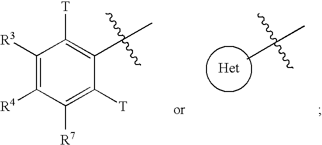
J represents N or CR¹;
K represents O, S, or NR¹;
Het represents a 5-6 membered nitrogen containing heterocycle substituted with 0 to 4 groups of R²;
T represents hydrogen, halogen, OR¹ or C₁₋₆ alkyl;
M is a negative charge, H, or a pharmaceutically acceptable metal or ammonium salt, and provided that when W contains a moiety with multiple positive charges, there is an appropriate number of L^{⊖} present to provide overall neutrality;
R¹ independently represents hydrogen, or C₁₋₆ alkyl;
R² and R⁵ independently represent hydrogen, halogen, cyano, —OR¹ or C₁₋₆ alkyl;
R³, R⁴, and R⁷ independently represent hydrogen, halogen, cyano, —OR¹ C₁₋₆ alkyl, or —Xₘ—Yₘ—Z*ₘ—R⁸;
X and Y independently are bond, O, (C═O), SO₂, (CH₂)ₙ, —(CH₂)ₙNR¹C(O)—, —(CH₂)ₙS—, or —(CH₂)ₙNR^{x}—;
Z* is (CH₂)ₙ which may be substituted with one to four R^{b},
R⁶ represents C₆₋₁₀ aryl, or C₅₋₁₀ heteroaryl, said aryl and heteroaryl optionally substituted;
R⁸ represents H, halogen, —N(R^{c})₂, —C(O)R⁵, —NR¹(CH₂)ₙC₅₋₁₀ aryl, —NR¹(CH₂)ₙC₅₋₁₀ heterocyclyl, (CH₂)ₙC₅₋₁₀ heterocyclyl, or (CH₂)ₙC₅₋₁₀ aryl, said heterocyclyl and aryl optionally substituted with one to four R^{b};
each R^{b} independently represents halogen; —CN; —NO₂; phenyl; —NHSO₂R^{c}; —OR^{c}, —SR^{c}; —N(R^{c})₂; —N⁺(R^{c})₃; —C(O)N(R^{c})₂; —SO₂N(R^{c})₂; heteroaryl; heteroarylium; —CO₂R^{c}; —C(O)R^{c}; —OC(O)R^{c}; —NHC(O)R^{c}; —NHC(O)₂R^{c}; guanidinyl; carbamimidoyl or ureido, said phenyl and heteroaryl optionally substituted;
each R^{c} independently represents hydrogen, a —C₁₋₆ straight or branched-chain alkyl group, a —C₃-C₆ cycloalkyl group or C₆₋₁₀ aryl, said aryl optionally substituted with one to four groups of halogen; —CN; —NO₂; phenyl; —NHSO₂R^{j}; —OR¹, —SR^{j}; —N(R^{j})₂; —N⁺(R^{j})₃; —C(O)N(R^{j})₂; —SO₂N(R^{j})₂; heteroaryl; heteroarylium; formamidinyl, —CO₂R^{j}; —C(O)R^{j}; —OC(O)R^{j}, —NHC(O)R^{j}, —NHC(O)₂R^{j}; guanidinyl; carbamimidoyl or ureido, said phenyl and heteroaryl optionally substituted, wherein Rj is selected from the group consisting of hydrogen, a —C₁₋₆ straight or branched-chain alkyl group, a —C₃-C₆ cycloalkyl group or C₆₋₁₀ aryl;
R^{x} represents hydrogen or a C₁₋₈ straight- or branched-alkyl chain, optionally interrupted by one or two of O, S, SO, SO₂, NR^{w}, N⁺R^{c}R^{w}, or —C(O)—, said alkyl chain being unsubstituted or substituted with one to four of halogen, CN, NO₂, —N₃, OR^{w}, SR^{w}, SOR^{w}, SO₂R^{w}, NR^{c}R^{w}, N⁺(R^{c})₂R^{w}, Q, —C(O)—R^{w}, C(O)NR^{c}R^{w}, SO₂NR^{c}R^{w}, CO₂R^{w}, OC(O)R^{w}, OC(O)NR^{c}R^{w}, NR^{c}C(O)R^{w}, NR^{c}C(O)NR^{c}R^{w}, phenyl, napthyl, heteroaryl, or heterocyclic group said phenyl, heteroaryl, and heterocyclic group optionally substituted with from one to four R^{b} groups or with one to two C₁₋₃ straight- or branched-chain alkyl groups, said alkyl groups being unsubstituted or substituted with one to four R^{b} groups; and
each R^{w} independently represents hydrogen; —C₁₋₆ straight- or branched-chain alkyl, unsubstituted or substituted with one to four R^{b} groups, —C₃₋₆ cycloalkyl optionally substituted with one to four R^{b} groups; phenyl optionally substituted with one to four R^{b} groups, or heteroaryl optionally substituted with one to four R^{b} groups;
m represents 0 to 1; n represents 0 to 6; wherein it is understood that when a value is zero, a bond exists.

2. The compound according to claim 1 wherein at least one of R³, R⁴, and R⁷ is —Xₘ—Yₘ—Z*ₘ—R⁸.

3. The compound according to claim 2 wherein R³ and R⁴, both are —Xₘ—Yₘ—Z*ₘ—R⁸.

4. The compound according to claim 2 wherein X is O and Y is (CH₂)ₙNR¹CO⁻, —(CH₂)ₙS—, or (CH₂)ₙNR^{x}.

5. The compound according to claim 1 of structural formula II: 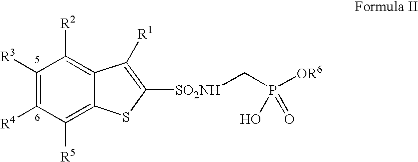
or a pharmaceutically acceptable salt thereof, wherein:
R¹, R², R³. R⁴, R⁵, R⁶ are as previously described.

6. A compound which is:
{2-[({[[4-cyano-3-(trifluoromethyl)phenoxy](hydroxy)phosphoryl]methyl}amino)sulfonyl]-4,7-difluoro-1-benzothiene-5,6-diyl}bis(oxyethane-2,1-diyliminocarbonylbenzene-4,1,2-triyl)tetraacetate;
{2-[({[[4-cyano-3-fluorophenoxy](hydroxy)phosphoryl]methyl}amino)sulfonyl]-4,7-difluoro-1-benzothiene-5,6-diyl}bis(oxyethane-2,1-diyliminocarbonylbenzene-4,1,2-triyl)tetraacetate;
{2-[({[[4-cyano-3-fluorophenoxy](hydroxy)phosphoryl]methyl}amino)sulfonyl]-4,7-dichloro-1-benzothiene-5,6-diyl}bis(oxyethane-2,1-diyliminocarbonylbenzene-4,1,2-triyl)tetraacetate;
{2-[({[(4-cyano-3-fluorophenoxy)(hydroxy)phosphoryl]methyl}amino)sulfonyl]-1-benzothiene-5,6-diyl}bis(oxyethane-2,1-diyliminocarbonylbenzene-3,1,2-triyl)tetraacetate;
{2-[({[[4-cyano-3-fluorophenoxy](hydroxy)phosphoryl]methyl}amino)sulfonyl]-1-benzothiene-5,6-diyl}bis(oxyethane-2,1-diyliminocarbonylbenzene-4,1,2-triyl)tetraacetate;
{2-[({[[4-cyano-3-(trifluoromethyl)phenoxy](hydroxy)phosphoryl]methyl}amino)sulfonyl]-1-benzothiene-5,6-diyl}bis(oxyethane-2,1-diyliminocarbonylbenzene-4,1,2-triyl)tetraacetate;
{2-[({[[(6-cyanopyridin-3-yl)oxy](hydroxy)phosphoryl]methyl}amino)sulfonyl]-1-benzothiene-5,6-diyl}bis(oxyethane-2,1-diyliminocarbonylbenzene-4,1,2-triyl)tetraacetate;
{2-[({[[4-cyano-3-fluorophenoxy] (hydroxy)phosphoryl]methyl}amino)sulfonyl]-1-benzothiene-5,6-diyl}bis(oxypropane-2,1-diyliminocarbonylbenzene-4,1,2-triyl)tetraacetate;
{2-[({[[4-cyano-3-(trifluoromethyl)phenoxy](hydroxy)phosphoryl]methyl}amino)sulfonyl]-1-benzothiene-5,6-diyl}bis(oxypropane-2,1-diyliminocarbonylbenzene-4,1,2-triyl)tetraacetate;
2-(acetyloxy)-4-({[3-({6-(4-{[3,4-bis(acetyloxy)benzoyl]amino}butoxy)-2-[({[(4-cyano-3-fluorophenoxy)-(hydroxy)phosphoryl]methyl}amino)sulfonyl]-1-benzothien-5-yl}oxy)propyl]amino}carbonyl)phenyl acetate;
2-(acetyloxy)-4-({[3-({6-(4-{[3,4-bis(acetyloxy)benzoyl]amino}propoxy)-2-[{([(4-cyano-3-fluorophenoxy)-(hydroxy)-phosphoryl]methyl}amino)sulfonyl]-1-benzothien-5-yl}oxy)butyl]amino}carbonyl)phenyl acetate;
{2-[({[[4-cyano-3-fluorophenoxy](hydroxy)phosphoryl]methyl}amino)sulfonyl]-4,7-difluoro-1-benzothiene-5,6-diyl}bis(oxypropane-2,1-diyliminocarbonylbenzene-4,1,2-triyl)tetraacetate;
{2-[({[[4-cyano-3-fluorophenoxy](hydroxy)phosphoryl]methyl}amino)sulfonyl]-3-methyl-1-benzothiene-5,6-diyl}bis(oxypropane-2,1-diyliminocarbonylbenzene-4,1,2-triyl)tetraacetate;
{2-[({[[4-cyano-3-fluorophenoxy](hydroxy)phosphoryl]methyl}amino)sulfonyl]-3-ethyl-1-benzothiene-5,6-diyl}bis(oxypropane-2,1-diyliminocarbonylbenzene-4,1,2-triyl)tetraacetate;
{2-[({[[4-cyano-3-fluorophenoxy](hydroxy)phosphoryl]methyl}amino)sulfonyl]-4,7-difluoro-3-ethyl-1-benzothiene-5,6-diyl}bis(oxypropane-2,1-diyliminocarbonylbenzene-4,1,2-triyl)tetraacetate;
{2-[({[[4-cyano-3-fluorophenoxy](hydroxy)phosphoryl]methyl}amino)sulfonyl]-4,7-difluoro-3-ethyl-1-benzothiene-5,6-diyl}bis(oxyethane-2,1-diyliminocarbonylbenzene-4,1,2-triyl)tetraacetate;
(4-{5-[({[(4-cyano-3-fluorophenoxy)(hydroxy)phosphoryl]methyl}amino)sulfonyl]-2-thienyl}-1,2-phenylene)bis(oxypropane-3,1-diyliminocarbonylbenzene-4,1,2-triyl)tetraacetate;
(4-{5-[({[(4-cyano-3-fluorophenoxy)(hydroxy)phosphoryl]methyl}amino)sulfonyl]-2-thienyl}-3-methyl-1,2-phenyl ene)bis(oxypropane-3,1-diyliminocarbonylbenzene-4,1,2-triyl)tetraacetate;
4-cyano-3-fluorophenyl hydrogen {[({5,6-bis[2-(1-naphthoylamino)ethoxy]-1-benzothien-2-yl}sulfonyl)amino]-methyl}phosphonate;
4-cyano-3-fluorophenyl hydrogen {[({5,6-bis[2-(2-naphthoylamino)ethoxy]-1-benzothien-2-yl}sulfonyl)amino]-methyl}phosphonate;
4-cyano-3-fluorophenyl hydrogen {[({5,6-bis[2-(benzoylamino)ethoxy]-1-benzothien-2-yl}sulfonyl)amino]-methyl}phosphonate;
4-cyano-3-fluorophenyl hydrogen {[({5,6-bis[2-(3,4-difluorobenzoylamino)ethoxy]-1-benzothien-2-yl}-sulfonyl)amino]methyl}phosphonate;
{2-[({[(4-cyano-3-fluorophenoxy)(hydroxy)phosphoryl]methyl}amino)sulfonyl]-1-benzothiene-5,6-diyl}bis(oxyethane-2,1-diyliminocarbonyl-4,1-phenylene) diacetate;
{2-[({[(4-cyano-3-fluorophenoxy)(hydroxy)phosphoryl]methyl}amino)sulfonyl]-1-benzothiene-5,6-diyl}bis(oxyethane-2,1-diyliminocarbonyl-3,1-phenylene) diacetate;
{2-[({[(4-cyano-3-fluorophenoxy)(hydroxy)phosphoryl]methyl}amino)sulfonyl]-1-benzothiene-5,6-diyl}bis[oxyethane-2,1-diylimino(2-oxoethane-2,1-diyl)benzene-4,1,2-triyl]tetraacetate;
4-(cyano-3-fluorophenyl hydrogen {5,6-bis[3-(3,4-dihydroxybenzamido)propoxy]benzo[d]thiazole-2-sulfonamido}-methylphosphonate;
4-cyano-3-(trifluoromethyl)phenyl hydrogen ({[(5,6-bis{2-[(3,4-dihydroxybenzoyl)amino]ethoxy}-4,7-difluoro-1-benzothien-2-yl)-sulfonyl]amino}methyl)phosphonate;
4-cyano-3-fluorophenyl hydrogen ({[(5,6-bis{2-[(3,4-dihydroxybenzoyl)amino]ethoxy}-4,7-difluoro-1-benzothien-2-yl)sulfonyl]amino}methyl)phosphonate;
4-cyano-3-fluorophenyl hydrogen ({[(5,6-bis{2-[(3,4-dihydroxybenzoyl)amino]ethoxy}-4,7-dichloro-1-benzothien-2-yl)sulfonyl]amino}methyl)phosphonate;
4-cyano-3-fluorophenyl hydrogen ({[(5,6-bis{2-[(2,3-dihydroxybenzoyl)amino]ethoxy}-1-benzothien-2-yl)-sulfonyl]amino}methyl)phosphonate;
4-cyano-3-(trifluoromethyl)phenyl hydrogen ({[(5,6-bis{2-[(2,3-dihydroxybenzoyl)amino]ethoxy}-1-benzothien-2-yl)-sulfonyl]amino}methyl)phosphonate;
4-cyano-3-fluorophenyl hydrogen ({[(5,6-bis{2-[(3,4-dihydroxybenzoyl)amino]ethoxy}-1-benzothien-2-yl)-sulfonyl]amino}methyl)phosphonate;
4-cyano-3-(trifluoromethyl)phenyl hydrogen ({[(5,6-bis{2-[(3,4-dihydroxybenzoyl)amino]ethoxy}-1-benzothien-2-yl)sulfonyl]amino}methyl)phosphonate;
3-fluoro-4-[(trifluoromethyl)sulfonyl]phenyl hydrogen ({[(5,6-bis{2-[(3,4-dihydroxybenzoyl)amino]ethoxy}-1-benzothien-2-yl)sulfonyl]amino}methyl)phosphonate;
6-cyanopyridin-3-yl hydrogen ({[(5,6-bis{2-[(3,4-dihydroxybenzoyl)amino]ethoxy}-1-benzothien-2-yl)sulfonyl]amino}methyl)phosphonate;
4-cyano-3-fluorophenyl hydrogen ({[(5,6-bis{2-[(3,4-dihydroxybenzoyl)amino]propoxy}-1-benzothien-2-yl)-sulfonyl]amino}methyl)phosphonate;
4-cyano-3-fluorophenyl hydrogen ({[(5,6-bis{2-[(3,4-dihydroxybenzoyl)amino]butoxy}-1-benzothien-2-yl)-sulfonyl]amino}methyl)phosphonate;
4-cyano-3-(trifluoromethyl)phenyl hydrogen ({[(5,6-bis{2-[(2,3-dihydroxybenzoyl)amino]propoxy}-1-benzothien-2-yl)sulfonyl]amino}methyl)phosphonate;
4-cyano-3-fluorophenyl hydrogen ({[(6-{4-[(3,4-dihydroxybenzoyl)amino]butoxy}-5-{3-[(3,4-dihydroxybenzoyl)amino]propoxy}-1-benzothien-2-yl)sulfonyl]amino}methyl)phosphonate;
4-cyano-3-fluorophenyl hydrogen ({[(5-{4-[(3,4-dihydroxybenzoyl)amino]butoxy}-6-{3-[(3,4-dihydroxybenzoyl)amino]propoxy}-1-benzothien-2-yl)sulfonyl]amino}methyl)phosphonate;
4-cyano-3-(trifluoromethyl)phenyl hydrogen ({[(5,6-bis{2-[(3,4-dihydroxybenzoyl)amino]propoxy}-4,7-dichloro-1-benzothien-2-yl)sulfonyl]amino}methyl)phosphonate;
4-cyano-3-fluorophenyl hydrogen ({[(5,6-bis{2-[(3,4-dihydroxybenzoyl)amino]propoxy}-4,7-difluoro-1-benzothien-2-yl)sulfonyl]amino}methyl)phosphonate;
4-cyano-3-fluorophenyl hydrogen ({[(5,6-bis{2-[(4-hydroxybenzoyl)amino]ethoxy}-1-benzothien-2-yl)-sulfonyl]amino}methyl)phosphonate;
4-cyano-3-fluorophenyl hydrogen ({[(5,6-bis{2-[(3-hydroxybenzoyl)amino]ethoxy}-1-benzothien-2-yl)-sulfonyl]amino}methyl)phosphonate;
4-cyano-3-fluorophenyl hydrogen ({[(5,6-bis{2-[3,4-dihydroxybenzoyl)amino]propoxy}-3-methyl-1-benzothien-2-yl)sulfonyl]amino}methyl)phosphonate;
4-cyano-3-fluorophenyl hydrogen ({[(5,6-bis{2-[(3,4-dihydroxybenzoyl)amino]propoxy}-3-ethyl-1-benzothien-2-yl)sulfonyl]amino}methyl)phosphonate;
4-cyano-3-fluorophenyl hydrogen ({[(5,6-bis{2-[(3,4-dihydroxybenzoyl)amino]propoxy}-4,7-difluoro-3-ethyl-1-benzothien-2-yl)sulfonyl]amino}methyl)phosphonate;
4-cyano-3-fluorophenyl hydrogen ({[(5,6-bis{2-[(3,4-dihydroxybenzoyl)amino]ethoxy}-4,7-difluoro-3-ethyl-1-benzothien-2-yl)sulfonyl]amino}methyl)phosphonate;
4-cyano-3-fluorophenyl hydrogen[({[5,6-bis(2-{[(3,4-dihydroxyphenyl)acetyl]amino}ethoxy)-1-benzothien-2-yl]sulfonyl}amino)methyl]phosphonate;
4-cyano-3-fluorophenyl hydrogen[({[5-(3,4-bis{3-[(3,4-dihydroxybenzoyl)amino]propoxy}phenyl)-2-thienyl]sulfonyl}amino)methyl]phosphonate;
4-cyano-3-fluorophenyl hydrogen[({[5-(3,4-bis{3-[(3,4-dihydroxybenzoyl)amino]propoxy}-2-methylphenyl)-2-thienyl]sulfonyl}amino)methyl]phosphonate;
4-Cyano-3-fluorophenyl hydrogen (5-{3,4-bis[3-(3,4-dihydroxybenzamido) propoxy]benzamido}-benzo[b]thiophene-2-sulfonamido)methylphosphonate;
4-cyano-3-fluorophenyl hydrogen (5-((pyridin-2-ylthio)methyl)thiophene-2-sulfonamido)-methylphosphonate;
4-cyano-3-fluorophenyl hydrogen (5-(dimethylamino)benzo[b]thiophene-2-sulfonamido)-methylphosphonate;
4-cyano-3-(trifluoromethyl)phenyl hydrogen (7-cyano-6-(dimethylamino)benzo[b]thiophene-2-sulfonamido)methylphosphonate;
4-cyano-3-(trifluoromethyl)phenyl hydrogen (5-(3,4-dihydroxybenzamido)benzo[b]thiophene-2-sulfonamido)methylphosphonate;
4-cyano-3-fluorophenyl hydrogen (5-(2,3-dihydroxybenzamido)benzo[b]thiophene-2-sulfonamido)methylphosphonate;
4-cyano-3-(trifluoromethyl)phenyl hydrogen (5-(2,3-dihydroxybenzamido)benzo[b]thiophene-2-sulfonamido)methylphosphonate;
4-cyano-3-fluorophenyl hydrogen (5-(3,4-dihydroxybenzamido)benzo[b]thiophene-2-sulfonamido)-methylphosphonate;
Ammounium 4-cyano-3-(trifluoromethyl)phenyl (2-(3,4-dimethoxyphenyl)thiazole-5-sulfonamido)methylphosphonate;
Ammonium 4-cyano-3-fluorophenyl (2-(3,4-dimethoxyphenyl)thiazole-5-sulfonamido)methylphosphonate;
4-cyano-3-fluorophenyl hydrogen (2-(3,4-dihydroxyphenyl)thiazole-5-sulfonamido)methylphosphonate;
4-cyano-3-(trifluoromethyl)phenyl hydrogen (2-(3,4-dihydroxyphenyl)thiazole-5-sulfonamido)methylphosphonate;
Ammonium 4-cyano-3-fluorophenyl (2-(2,3-dimethoxyphenyl)thiazole-5-sulfonamido)methylphosphonate;
Ammonium 4-cyano-3-fluorophenyl (5-(3,4-dimethoxyphenyl)thiophene-2-sulfonamido)methylphosphonate;
Ammonium 4-cyano-3-(trifluoromethyl)phenyl (5-(3,4-dimethoxyphenyl)thiophene-2-sulfonamido)methylphosphonate;
4-cyano-3-(trifluoromethyl)phenyl hydrogen (5-(3,4-dihydroxyphenyl)thiophene-2-sulfonamido)methylphosphonate;
4-cyano-3-fluorophenyl hydrogen (5-(3,4-dihydroxyphenyl)thiophene-2-sulfonamido)methylphosphonate;
Ammonium 4-cyano-3-fluorophenyl (5-(6-methoxypyridin-3-yl)thiophene-2-sulfonamido)methylphosphonate;
Ammonium 4-cyano-3-(trifluoromethyl)phenyl (5-(6-methoxypyridin-3-yl)thiophene-2-sulfonamido)methylphosphonate;
6-cyanopyridin-3-yl hydrogen (5-(6-methoxypyridin-3-yl)thiophene-2-sulfonamido)methylphosphonate;
4-cyano-3-fluorophenyl hydrogen (2-(2,3-dihydroxyphenyl)thiazole-5-sulfonamido)methylphosphonate;
4-cyano-3-(trifluoromethyl)phenyl hydrogen (2-(2,3-dimethoxyphenyl)thiazole-5-sulfonamido)methylphosphonate;
4-cyano-3-(trifluoromethyl)phenyl hydrogen (2-(2,3-dihydroxyphenyl)thiazole-5-sulfonamido)methylphosphonate;
4-cyano-3-fluorophenyl hydrogen (5-(6-oxo-1,6-dihydropyridin-3-yl)thiophene-2-sulfonamido)methylphosphonate;
Ammonium 4-cyano-3-fluorophenyl (5-(2,3-dimethoxyphenyl)thiophene-2-sulfonamido)methylphosphonate;
Ammonium 4-cyano-3-(trifluoromethyl)phenyl (5-(2,3-dimethoxyphenyl)thiophene-2-sulfonamido)methylphosphonate;
4-cyano-3-fluorophenyl hydrogen (5-(2,3-dihydroxyphenyl)thiophene-2-sulfonamido)methylphosphonate;
4-cyano-3-(trifluoromethyl)phenyl hydrogen (5-(2,3-dihydroxyphenyl)thiophene-2-sulfonamido)methylphosphonate;
Ammonium 4-cyano-3-(trifluoromethyl)phenyl (5,6-dimethoxybenzofuran-2-sulfonamido)methylphosphonate;
4-cyano-3-(trifluoromethyl)phenyl hydrogen (5,6-dihydroxybenzofuran-2-sulfonamido)methylphosphonate; and
Ammonium 4-cyano-3-fluorophenyl(5-((3,4-dihydroxyphenylsulfonyl)methyl)thiophene-2-sulfonamido)methylphosphonate
or pharmaceutically acceptable salts thereof.

7. A pharmaceutical composition which is comprised of a compound in accordance with claim 1 and a pharmaceutically acceptable carrier.

8. The composition according to claim 7 in combination with a beta-lactam antibiotic selected from the group consisting of imipenem, Primaxin®, Amoxicillin, Ticarcillin, Ampicillin, Cefoperazone, Piperacillin, and ceftazidime.

9. The composition of claim 8 wherein the beta-lactam antibiotic is Primaxin®.

10. Use of a compound of formula I of claim 1 in combination with a beta lactam antibiotic in the manufacture of a medicament for treating bacterial infections or for inhibiting beta-lactamase.
